(19) United States
(12) Patent Application Publication
(10) Pub. No.: US 2021/0087184 A1
(43) Pub. Date: Mar. 25, 2021

(54) AMINO-BENZOISOTHIAZOLE AND AMINO-BENZOISOTHIADIAZOLE AMIDE COMPOUNDS

(71) Applicant: Aerie Pharmaceuticals, Inc., Bedminster, NJ (US)

(72) Inventors: Mitchell A. deLong, Chapel Hill, NC (US); Jill M. Sturdivant, Chapel Hill, NC (US); Cynthia L. Lichorowic, Raleigh, NC (US)

(21) Appl. No.: 16/977,929

(22) PCT Filed: Mar. 14, 2019
(86) PCT No.: PCT/US2019/022204

§ 371 Date: Sep. 3, 2020

Related U.S. Application Data
(60) Provisional application No. 62/760,592, filed on Nov. 13, 2018.
(60) Provisional application No. 62/738,940, filed on Sep. 28, 2018.
(60) Provisional application No. 62/643,129, filed on Mar. 14, 2018.

(51) Int. Cl. C07D 417/12; C07D 417/14; C07D 275/04; C07D 285/14; A61K 9/00; A61P 27/06; A61K 47/36; A61K 47/38; A61K 47/02; A61K 47/18

(52) U.S. Cl. CPC C07D 417/12; C07D 417/14; C07D 275/04; C07D 285/14; A61K 9/0048; A61K 47/186; A61K 47/36; A61K 47/38; A61K 47/02; A61K 47/183; A61P 27/06

(57) ABSTRACT

Provided herein are amino-benzoisothiazole and benzoisothiadiazole amide compounds. In particular, provided herein are compounds that affect the function of kinases in a cell and that are useful as therapeutic agents or with therapeutic agents. The compounds provided herein are useful in the treatment of a variety of diseases and conditions including eye diseases such as glaucoma, retinal diseases such as acute macular degeneration (AMD) and diabetic macular edema (DME), diseases and conditions characterized by inflammatory processes, cardiovascular diseases, and diseases characterized by abnormal growth, such as cancers. Also provided are compositions (e.g., pharmaceutical compositions) comprising the compounds provided herein.

### RELATED APPLICATIONS

This application is a national stage entry under 35 U.S.C. § 371 of International Patent Application No. PCT/US2019/022204, filed Mar. 14, 2019, which claims priority to U.S. Provisional Patent Application No. 62/760,592, filed Nov. 13, 2018, U.S. Provisional Patent Application No. 62/738,940, filed Sep. 28, 2018, and U.S. Provisional Patent Application No. 62/643,129, filed Mar. 14, 2018, the entire content of each of which is incorporated herein by reference.

### FIELD OF THE DISCLOSURE

The present disclosure relates to amino-benzoisothiazole and benzoisothiadiazole amide compounds that affect the function of kinases in a cell and that are useful as therapeutic agents or with therapeutic agents. In particular, these compounds are useful in the treatment of eye diseases such as glaucoma, DME, AMD, and diabetic retinopathy, for the treatment of inflammatory disease, for the treatment of cardiovascular diseases, and for diseases characterized by abnormal growth, such as cancers.

### BACKGROUND

A variety of hormones, neurotransmitters and biologically active substances control, regulate or adjust the functions of living bodies via specific receptors located in cell membranes. Many of these receptors mediate the transmission of intracellular signals by activating guanine nucleotide-binding proteins (G proteins) to which the receptor is coupled. Such receptors are generically referred to as G-protein coupled receptors (GPCRs) and include, among others, α-adrenergic receptors, β-adrenergic receptors, opioid receptors, cannabinoid receptors and prostaglandin receptors. The biological effects of activating these receptors is not direct but is mediated by a ‘downstream’ host of intracellular proteins. One class of these downstream effectors is the “kinase” class.

The various kinases play roles in the regulation of various physiological functions. For example, kinases have been implicated in a number of disease states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis. Other conditions include chronic inflammatory bowel disease, glaucoma, hypergastrinemia, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (e.g., cardiac malformations), neurodegenerative diseases such as Parkinson's Disease and Alzheimer's Disease, multiple sclerosis, Epstein-Barr infection and cancer. In other disease states, the role of kinases is only now becoming clear. The retina is a complex tissue composed of multiple interconnected cell layers, highly specialized for transforming light and color into electrical signals that are perceived by the brain. Damage or death of the primary light-sensing cells, the photoreceptors, results in devastating effects on vision. Despite the identification of numerous mutations that cause inherited retinal degenerations, the cellular and molecular mechanisms leading from the primary mutations to photoreceptor apoptosis are not well understood.

The balance between the initiation and the inactivation of intracellular signals determines the intensity and duration of the response of the receptors to stimuli such as agonists. When desensitization occurs, the mediation or regulation of the physiological function mediated or regulated by the G proteins to which the receptors are coupled is reduced or prevented. For example, when agonists are administered to treat a disease or condition by activation of certain receptors, the receptors relatively quickly become desensitized from the action of the GRKs such that agonist administration may no longer result in therapeutic activation of the appropriate receptors. At that point, administration of the agonist no longer enables sufficient or effective control of or influence on the disease or condition intended to be treated.

In view of the role that kinases have in many disease states, there is an urgent and continuing need for small molecule ligands which inhibit or modulate the activity of kinases. Without wishing to be bound by theory, it is thought that modulation of the activity of kinases by the compounds of the present disclosure is responsible for their beneficial effects.

### SUMMARY

In one aspect, provided herein are compounds of Formula I:

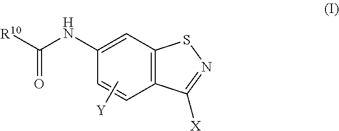

or a pharmaceutically acceptable salt thereof;

wherein

R¹⁰ is C₁-C₁₀ alkyl, C₂-C₁₀ alkenyl, C₂-C₁₀ alkynyl, an alkylaryl group, an aryl group, an alkylheteroaryl group, a heteroaryl group, an alkylcycloalkyl group, a cycloalkyl group, an alkylheterocycloalkyl group, a heterocycloalkyl group, each of which may be independently further substituted, the stereocenters being either ‘rac,’ ‘R’ or ‘S’ in configuration independently; and

X and Y are, independently, hydrogen, hydroxyl, halogen, C₁-C₄ alkyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In another aspect, provided herein are compounds of Formula II:

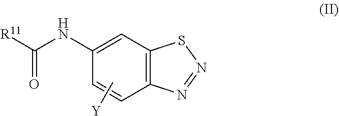

or a pharmaceutically acceptable salt thereof;

wherein

R¹¹ is C₁-C₁₀ alkyl, C₂-C₁₀ alkenyl, C₂-C₁₀ alkynyl, an alkylaryl group, an aryl group, an alkylheteroaryl group, a heteroaryl group, an alkylcycloalkyl group, a cycloalkyl group, an alkylheterocycloalkyl group, a heterocycloalkyl group, each of which may be independently further substituted, the stereocenters being either ‘rac,’ ‘R’ or ‘S’ in configuration independently; and

Y is hydrogen, hydroxyl, halogen, C₁-C₄ alkyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In yet another aspect, provided herein are compounds of Formula III:

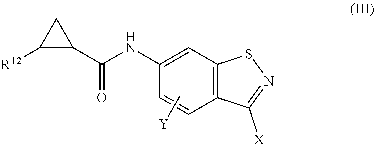

or a pharmaceutically acceptable salt thereof;

wherein

R¹² is C₁-C₁₀ alkyl, C₂-C₁₀ alkenyl, C₂-C₁₀ alkynyl, an alkylaryl group, an aryl group, an alkylheteroaryl group, a heteroaryl group, an alkylcycloalkyl group, a cycloalkyl group, an alkylheterocycloalkyl group, a heterocycloalkyl group, each of which may be independently further substituted, the stereocenters being either ‘rac,’ ‘R’ or ‘S’ in configuration independently; and

X and Y are, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In still another aspect, provided herein are compounds of Formula IV:

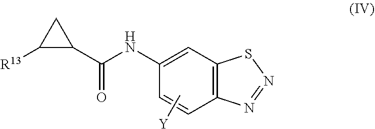

or a pharmaceutically acceptable salt thereof;

wherein

R¹³ is C₁-C₁₀ alkyl, C₂-C₁₀ alkenyl, C₂-C₁₀ alkynyl, an alkylaryl group, an aryl group, an alkylheteroaryl group, a heteroaryl group, an alkylcycloalkyl group, a cycloalkyl group, an alkylheterocycloalkyl group, a heterocycloalkyl group, each of which may be independently further substituted, the stereocenters being either ‘rac,’ ‘R’ or ‘S’ in configuration independently;

Y is hydrogen, hydroxyl, halogen, C₁-C₄ alkyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In yet another aspect, provided herein are compounds of Formula (V):

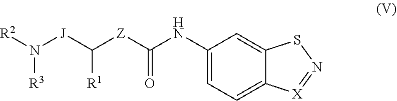

or a pharmaceutically acceptable salt thereof;

wherein

X is C—R⁶ or N;

J is a bond, methylene or ethylene;

Z is a bond, methylene or ethylene;

R¹ is hydrogen; halogen; —C₁₋₆-alkyl; —C₁₋₆-haloalkyl, —(C₁₋₆-alkyl)-OH; —C₆₋₁₀-aryl; heteroaryl; —CH₂-heteroaryl; —CH₂—(C₆₋₁₀-aryl); —C₃₋₁₀-cycloalkyl; —CH₂—(C₃₋₁₀-cycloalkyl); —C(O)N(H)—(C₆₋₁₀-aryl); —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl) substituted with halogen, —C₁₋₆-alkyl, —C₁₋₆-haloalkyl, or —(C₁₋₆-alkyl)-OH;

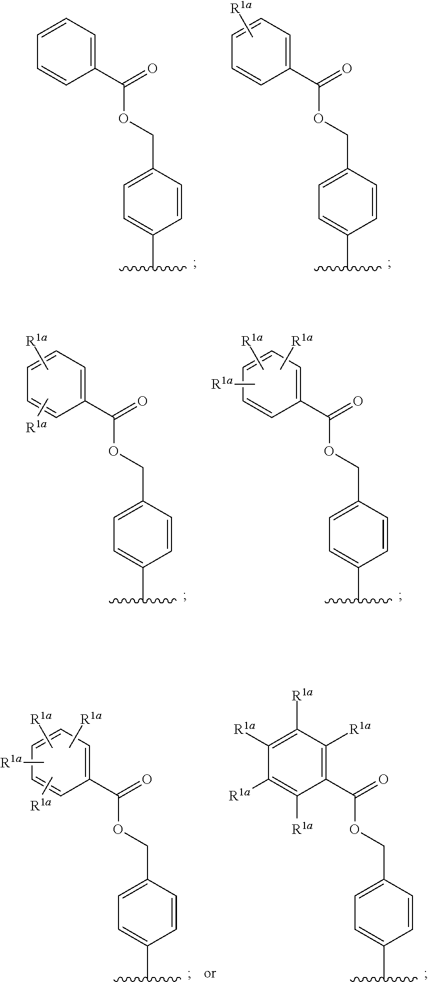

R^{1a} is, independently, halogen, —C₁₋₆-alkyl, or —C₁₋₆-haloalkyl;

R² is hydrogen, —C₁₋₆-alkyl, —CH₂—(C₆₋₁₀-aryl), —(C₁₋₆-alkyl)N(C₁₋₆-alkyl)C₁₋₆-alkyl, or —C(NH)NH₂;

R³ is hydrogen or —C₁₋₆-alkyl;

or R² and R³, together with the atoms to which they are attached, form a C₂₋₆-heterocycloalkyl;

or Z is CH and R³ and Z, together with the atoms to which they are attached, form a C₃₋₆-heterocycloalkyl; and R⁶ is hydrogen, —C₁₋₆-alkyl, —OH, —CN, —O—(C₁₋₆-alkyl), —C(H)(F)—CH₃, or —C₁₋₆-haloalkyl.

In still another aspect, provided herein are compounds of Formula (VI).

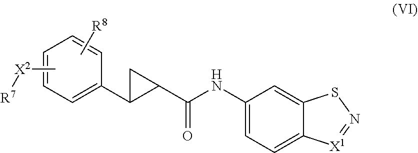

or a pharmaceutically acceptable salt thereof;

wherein

X¹ is C—R⁶ or N;

X² is —C(O)— or —SO₂—;

R⁷ is —OH; —NH₂; —O—(C₁₋₆-alkyl); —N(H)—(C₁₋₃-alkyl)-heteroaryl; —N(H)— heteroaryl; —N(H)—(C₁₋₃-alkyl)-(C₆₋₁₀-aryl)-(C₁₋₃-alkyl)-N(C₁₋₃-alkyl)₂; —N(H)—(C₆₋₁₀-aryl)-(C₁₋₃-alkyl)-N(C₁₋₃-alkyl)₂; —N(H)—(C₁₋₃-alkyl)-heteroalkyl; —N(H)—(C₁₋₃-alkyl)-N(C₁₋₃-alkyl)₂; heteroalkyl; -heteroalkyl-(C₆₋₁₀-aryl); —N(H)-heteroalkyl; heteroalkyl; —N(H)—(C₁₋₆-alkyl); —O—(C₁₋₃-alkyl)-heteroaryl; —O—(C₁₋₃-alkyl)-(C₆₋₁₀-aryl) substituted with —C₁₋₃-alkyl or C₁₋₃-haloalkyl; —O—(C₁₋₃-alkyl)-heteroalkyl; —O—(C₁₋₃-alkyl)-N(C₁₋₃-alkyl)₂; —O—(C₁₋₃-akyl)-(C₆₋₁₀-aryl); or —O—(C₁₋₆-alkyl);

R⁶ is hydrogen or —OH; and

R⁸ is hydrogen or halogen.

In another aspect, provided herein are compositions comprising a compound of the Formulae provided herein.

In yet another aspect, provided herein are pharmaceutical compositions comprising a compound of the Formulae provided herein and a pharmaceutically acceptable carrier.

In still another aspect, provided herein are kits comprising a compound of the Formulae provided herein and instructions for use thereof.

In another aspect, provided herein are articles of manufacture comprising a compound of the Formulae provided herein.

In yet another aspect, provided herein are methods of treating a disease in a subject in need thereof, comprising administering to the subject an effective amount of a compound of the Formulae provided herein.

In still another aspect, provided herein are methods of modulating kinase activity in a cell, comprising contacting the cell with a compound of the Formulae provided herein in an amount effective to modulate kinase activity.

In another aspect, provided herein are methods of reducing intraocular pressure in a subject in need thereof, comprising contacting the subject with an effective amount of a compound of the Formulae provided herein.

### DETAILED DESCRIPTION

Publications and patents are referred to throughout this disclosure. All U.S. patents cited herein are hereby incorporated by reference. All percentages, ratios, and proportions used herein are percent by weight unless otherwise specified.

6-amino-benzoisothiazole amides and 6-amino-benzoisothiadiazole amides are provided herein.

“Alkyl” refers to a saturated aliphatic hydrocarbon including straight chain and branched chain groups. “Alkyl” may be exemplified by groups such as methyl, ethyl, n-propyl, isopropyl, n-butyl and the like. Alkyl groups may be substituted or unsubstituted. Substituents may also be themselves substituted. When substituted, the substituent group is preferably but not limited to C₁-C₄ alkyl, aryl, amino, cyano, halogen, alkoxy, fluoro, or hydroxyl. “C₁-C₄ alkyl” refers to alkyl groups containing one to four carbon atoms.

“Alkenyl” refers to an unsaturated aliphatic hydrocarbon moiety including straight chain and branched chain groups. Alkenyl moieties must contain at least one alkene. “Alkenyl” may be exemplified by groups such as ethenyl, n-propenyl, isopropenyl, n-butenyl and the like. Alkenyl groups may be substituted or unsubstituted. Substituents may also be themselves substituted. When substituted, the substituent group is preferably alkyl, halogen or alkoxy. Substituents may also be themselves substituted. Substituents may be placed on the alkene itself and also on the adjacent member atoms or the alkynyl moiety. “C₂-C₄ alkenyl” refers to alkenyl groups containing two to four carbon atoms.

“Alkynyl” refers to an unsaturated aliphatic hydrocarbon moiety including straight chain and branched chain groups. Alkynyl moieties must contain at least one alkyne. “Alkynyl” may be exemplified by groups such as ethynyl, propynyl, n-butynyl and the like. Alkynyl groups may be substituted or unsubstituted. When substituted, the substituent group is preferably alkyl, amino, cyano, halogen, alkoxyl or hydroxyl. Substituents may also be themselves substituted. Substituents are not on the alkyne itself but on the adjacent member atoms of the alkynyl moiety. “C₂-C₄ alkynyl” refers to alkynyl groups containing two to four carbon atoms.

“Acyl” or “carbonyl” refers to the group —C(O)R wherein R is alkyl; alkenyl; alkynyl, aryl, heteroaryl, carbocyclic, heterocarbocyclic; alkylaryl or alkylheteroaryl. Alkylcarbonyl refers to a group wherein the carbonyl moiety is preceded by an alkyl chain of 1-4 carbon atoms.

“Alkoxy” refers to the group —O—R wherein R is acyl, alkyl alkenyl, alkyl alkynyl, aryl, carbocyclic; heterocarbocyclic; heteroaryl, C₁-C₄ alkyl aryl or C₁-C₄ alkyl heteroaryl.

“Amino” refers to the group —NR′R′ wherein each R′ is, independently, hydrogen, amino, hydroxyl, alkoxyl, alkyl, aryl, cycloalkyl, heterocycloalkyl, heteroaryl, C₁-C₄ alkyl aryl or C₁-C₄ alkyl heteroaryl. The two R′ groups may themselves be linked, together with the nitrogen to which they are attached, to form a ring. The R′ groups may themselves be further substituted, in which case the group also known as guanidinyl is specifically contemplated under the term ‘amino”.

“Aryl” refers to an aromatic carbocyclic group. “Aryl” may be exemplified by phenyl. The aryl group may be substituted or unsubstituted. Substituents may also be themselves substituted. When substituted, the substituent group is preferably but not limited to heteroaryl; acyl, carboxyl, carbonylamino, nitro, amino, cyano, halogen, or hydroxyl.

“Benzoisothiazole” refers to the bicyclic heteroaronatic ring structure:

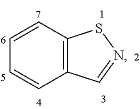

with the systematic numbering as shown. In some publications this structure is referred to as “1,2-benzisothiazole”, or “benz[d]isothiazole.

“Benzoisothiadiazole” refers to the bicyclic heteroaromatic ring structure:

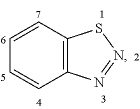

with the systematic numbering as shown.

“Carboxyl” refers to the group —C(═O)O—C₁-C₄ alkyl or aryl.

“Carbonyl” refers to the group —C(O)R wherein each R is, independently, hydrogen, alkyl, aryl, cycloalkyl; heterocycloalkyl; heteroaryl, C₁-C₄ alkyl aryl or C₁-C₄ alkyl heteroaryl.

“Carbonylamino” refers to the group —C(O)NR′R′ wherein each R′ is, independently, hydrogen, alkyl, alkoxy, aryl, cycloalkyl; heterocycloalkyl; heteroaryl, C₁-C₄ alkylaryl or C₁-C₄ alkylheteroaryl. The two R′ groups may themselves be linked, together with the nitrogen to which they are attached, to form a ring.

“Alkylaryl” refers to alkyl groups having an aryl substituent such that the aryl substituent is bonded through an alkyl group. “Alkylaryl” may be exemplified by benzyl.

“Alkylheteroaryl” refers to alkyl groups having a heteroaryl substituent such that the heteroaryl substituent is bonded through an alkyl group.

“Carbocyclic group” or “cycloalkyl” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the ring. Carbocyclic groups may be substituted or unsubstituted. Substituents may also be themselves substituted. Preferred carbocyclic groups include cyclopropyl, cyclobutyl, cyclopentyl, cyclohexyl, cyclohexenyl, and cycloheptyl. More preferred carbocyclic groups include cyclopropyl and cyclobutyl. The most preferred carbocyclic group is cyclopropyl. Carbocyclic groups are not aromatic.

“Halogen” refers to fluoro, chloro, bromo or iodo moieties. Preferably, the halogen is fluoro, chloro, or bromo.

“Heteroaryl” or “heteroaromatic” refers to a monocyclic or bicyclic aromatic carbocyclic radical having one or more heteroatoms in the carbocyclic ring. Heteroaryl may be substituted or unsubstituted. When substituted, the substituents may themselves be substituted. Preferred but non limiting substituents are aryl; C₁-C₄ alkylaryl; amino; halogen, hydroxy, cyano, nitro; carboxyl; carbonylamino or C₁-C₄ alkyl. Preferred heteroaromatic groups include isoquinolinyl, benzoisothiazolyl, benzoisothiadiazolyl, benzothiofuranyl, thienyl, furanyl, tetrazoyl, triazolyl, and pyridyl.

“Heteroatom” means an atom other than carbon in the ring of a heterocyclic group or a heteroaromatic group or the chain of a heterogeneous group. Preferably, heteroatoms are selected from the group consisting of nitrogen, sulfur, and oxygen atoms. Groups containing more than one heteroatom may contain different heteroatoms.

“Heterocarbocyclic group” or “heterocycloalkyl” or “heterocyclic” means a monovalent saturated or unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, preferably 4 to 7 carbon atoms, and more preferably 5 to 6 carbon atoms in the ring. Bicyclic heterocarbocyclic groups contain 8 to 12 carbon atoms, preferably 9 to 10 carbon atoms in the ring. Heterocarbocyclic groups may be substituted or unsubstituted. Substituents may also be themselves substituted. Preferred heterocarbocyclic groups include epoxy, tetrahydrofuranyl, azacyclopentyl, azacyclohexyl, piperidyl, and homopiperidyl. More preferred heterocarbocyclic groups include piperidyl, and homopiperidyl. The most preferred heterocarbocyclic group is piperidyl. Heterocarbocyclic groups are not aromatic.

“Hydroxy” or “hydroxyl” means a chemical entity that consists of —OH. Alcohols contain hydroxy groups. Hydroxy groups may be free or protected. An alternative name for hydroxyl is hydroxy. A hydroxyl group at C3 of a benzoisothiazole can also be drawn in its tautomeric form.

“Linker” means a linear chain of n member atoms where n is an integer of from 1 to 4.

“Member atom” means a carbon, nitrogen, oxygen or sulfur atom. Member atoms may be substituted up to their normal valence. If substitution is not specified the substituents required for valency are hydrogen.

“Ring” means a collection of member atoms that are cyclic. Rings may be carbocyclic, aromatic, or heterocyclic or heteroaromatic, and may be substituted or unsubstituted, and may be saturated or unsaturated. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Thioalkyl” refers to the group —S-alkyl.

“Sulfonyl” refers to the —S(O)₂R′ group wherein R′ is alkoxy, alkyl, aryl, carbocyclic, heterocarbocyclic; heteroaryl, C₁-C₄ alkyl aryl or C₁-C₄ alkyl heteroaryl.

“Sulfonylamino” refers to the —S(O)₂NR′R′ group wherein each R′ is independently hydrogen, alkyl, aryl, heteroaryl, C₁-C₄ alkyl aryl or C₁-C₄ alkyl heteroaryl.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal and oral administration. “Pharmaceutically acceptable carrier” also includes agents for preparation of aqueous dispersions and sterile powders for injection or dispersions.

As used herein, “pharmaceutically acceptable salts” refers to an ionizable therapeutic agent that has been combined with a counter-ion to form a neutral complex. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

“Excipient” as used herein includes physiologically compatible additives useful in preparation of a pharmaceutical composition. Examples of pharmaceutically acceptable carriers and excipients can for example be found in Remington Pharmaceutical Science, 16^{th} Ed.

“Therapeutically effective amount” as used herein refers to a dosage of the compounds or compositions effective for influencing, reducing or inhibiting the activity of or preventing activation of a kinase. This term as used herein may also refer to an amount effective at bringing about a desired in vivo effect in an animal, preferably, a human, such as reduction in intraocular pressure.

“Administering” as used herein refers to administration of the compounds as needed to achieve the desired effect.

“Eye disease” as used herein includes, but is not limited to, glaucoma, allergy, cancers of the eye, neurodegenerative diseases of the eye such as DME and AMD, and dry eye.

The term “disease or condition associated with kinase activity” is used to mean a disease or condition treatable, in whole or in part, by inhibition of one or more kinases.

The term “controlling the disease or condition” is used to mean changing the activity of one or more kinases to affect the disease or condition.

The term “contacting a cell” is used to mean contacting a cell in vitro or in vivo (i.e. in a subject, such as a mammal, including humans, cats and dogs).

### Compounds/Compositions

The Benzoisothiazole compounds may be represented by Formula I and Formula III:

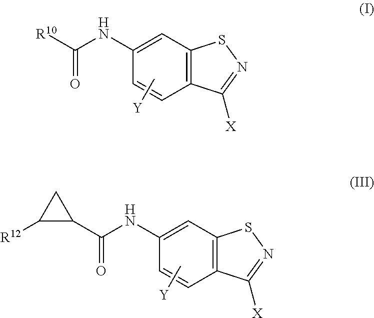

wherein R¹⁰ and R¹² are, independently, hydrogen, C₁-C₄ alkyl, C₂-C₄ alkenyl, C₂-C₄ alkynyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl; an aryl group, a heteroaryl group, a cycloalkyl group, a heterocycloalkyl group, C₁-C₈ alkyl, C₂-C₈ alkenyl, C₂-C₈ alkynyl, the stereocenters being either ‘R’ or ‘S’ in configuration independently,

wherein X and Y are, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In some embodiments, X and Y are, independently, hydrogen, hydroxyl, halogen, C₁-C₄ alkyl, C₂-C₄ alkenyl, C₂-C₄ alkynyl, amino, nitro, cyano, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In some embodiments of Formula I, R¹⁰ is an alkylaryl group or an alkylheteroaryl group with a pendant amine, and Y and X are hydrogen. In another embodiment of Formula I, R¹⁰ is an alkylcycloalkyl group, Y is F, and X is a hydrogen group.

In some embodiments of Formula III, R¹² is an aryl group or an alkylheteroaryl group with para sulfonamide moiety, and X and Y are hydrogen. In another embodiment of Formula III, R¹² is an aryl group or an alkylheteroaryl group with meta amide moiety, and Y and X are hydrogen.

The benzoisothiadiazole compounds may be represented by Formula II and Formula IV:

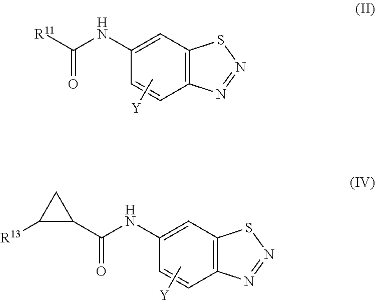

wherein R¹¹ and R¹³ are, independently, hydrogen, C₁-C₄ alkyl, C₂-C₄ alkenyl, C₂-C₄ alkynyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl, an aryl group, a heteroaryl group, a cycloalkyl group, a heterocycloalkyl group, C₁-C₈ alkyl, C₂-C₈ alkenyl, C₂-C₈ alkynyl, the stereocenters being either ‘R’ or ‘S’ in configuration independently,

wherein Y is, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In some embodiments, Y is, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

In some embodiments of Formula II, R¹¹ is an alkylaryl group or an alkylheteroaryl group with a pendant amine, and Y is hydrogen. In another preferred embodiment of Formula II, R¹¹ is an alkylcycloalkyl group, and Y is F.

In some embodiments of Formula IV, R¹³ is an aryl group or an alkylheteroaryl group with para sulfonamide moiety, and Y is hydrogen. In another preferred embodiment of Formula IV, R¹³ is an aryl group or an alkylheteroaryl group with meta amide moiety, and Y is hydrogen.

In one aspect, provided herein is a compound of Formula (V):

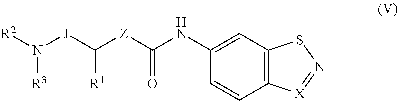

or a pharmaceutically acceptable salt thereof;

wherein

X is C—R⁶ or N;

J is a bond, methylene or ethylene;

Z is a bond, methylene or ethylene;

R¹ is hydrogen; halogen; —C₁₋₆-alkyl; —C₁₋₆-haloalkyl, —(C₁₋₆-alkyl)-OH; —C₆₋₁₀-aryl; heteroaryl; —CH₂-heteroaryl; —CH₂—(C₆₋₁₀-aryl); —C₃₋₁₀-cycloalkyl; —CH₂—(C₃₋₁₀-cycloalkyl); —C(O)N(H)—(C₆₋₁₀-aryl); —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl) substituted with halogen, —C₁₋₆-alkyl, —C₁₋₆-haloalkyl, or —(C₁₋₆-alkyl)-OH;

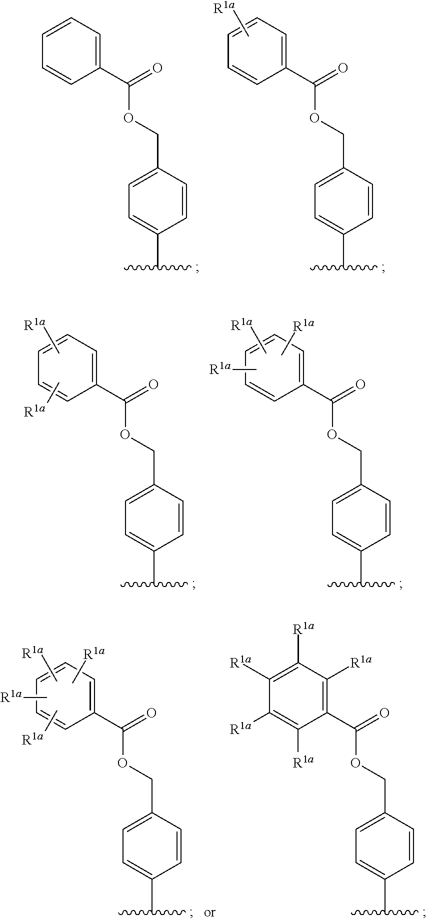

R^{1a} is, independently, halogen, —C₁₋₆-alkyl, or —C₁₋₆-haloalkyl;

R² is hydrogen, —C₁₋₆-alkyl, —CH₂—(C₆₋₁₀-aryl), —(C₁₋₆-alkyl)N(C₁₋₆-alkyl)C₁₋₆-alkyl, or —C(NH)NH₂;

R³ is hydrogen or —C₁₋₆-alkyl;

or R² and R³, together with the atoms to which they are attached, form a C₂₋₆-heterocycloalkyl;

or Z is CH and R³ and Z, together with the atoms to which they are attached, form a C₃₋₆-heterocycloalkyl;

R⁶ is hydrogen, —C₁₋₆-alkyl, —OH, —CN, —O—(C₁₋₆-alkyl), —C(H)(F)—CH₃, or —C₁₋₆-haloalkyl.

In some embodiments, the compound of Formula (V) is a compound of Formula (VI):

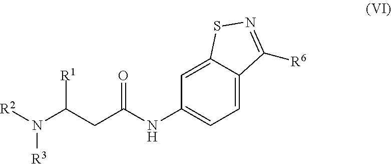

or a pharmaceutically acceptable salt thereof;

wherein

R¹ is —C₁₋₆-alkyl; —C₆₋₁₀-aryl; —C₆₋₁₀-aryl monosubstituted with halogen, —C₁₋₆-alkyl or hydroxymethyl; —C₆₋₁₀-aryl disubstituted with halogen, —C₁₋₆-alkyl or hydroxymethyl; heteroaryl; —CH₂-heteroaryl; —CH₂—(C₆₋₁₀-aryl); —CH₂—(C₆₋₁₀-aryl) monosubstituted with halogen; —C₃₋₁₀-cycloalkyl; or —CH₂—(C₃₋₁₀-cycloalkyl);

R² is hydrogen or —C₁₋₆-alkyl;

R³ is hydrogen or —C₁₋₆-alkyl; and

R⁶ is hydrogen or —OH.

In some embodiments of the Formulae provided herein, X and Y are, independently, H, methyl, F, or Cl. In some embodiments, X and Y are, independently, methyl, F, or Cl.

In some embodiments of the Formulae provided herein, R¹² and R¹³ are, independently, phenyl or 6-membered heteroaryl (i.e. pyridinyl or pyrimidinyl).

In some embodiments, R² is hydrogen or methyl.

In some embodiments, R³ is hydrogen or methyl.

In some embodiments, R¹ is methyl; phenyl; phenyl monosubstituted with halogen, methyl or hydroxymethyl; phenyl disubstituted with halogen, methyl or hydroxymethyl; thienyl; —CH₂-thienyl; furyl; pyridyl; benzyl; benzyl monosubstituted with halogen; cyclohexyl; cyclopropyl; —CH₂-cyclohexyl; thiazole; oxazole; or piperidyl.

In some embodiments, the compound of Formula (V) is a compound of Formula (V2):

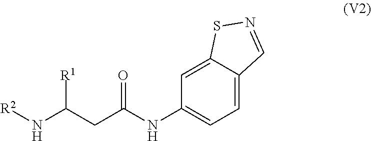

or a pharmaceutically acceptable salt thereof.

In some embodiments, R² is hydrogen.

In some embodiments, the compound is a compound of Table 1, or a pharmaceutically acceptable salt thereof.

In some embodiments, the compound of Formula (V) is a compound of Formula (V3):

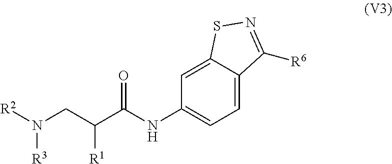

or a pharmaceutically acceptable salt thereof;

wherein

R⁶ is hydrogen, methyl, —OH, —CN, —OCH₃, —C(H)(F)—CH₃, or —CH₂F;

R¹ is —C₆₋₁₀-aryl; —C₆₋₁₀-aryl monosubstituted with halogen, —C₁₋₆-alkyl or hydroxymethyl; heteroaryl; —C₃₋₁₀-cycloalkyl; —C(O)N(H)—(C₆₋₁₀-aryl);

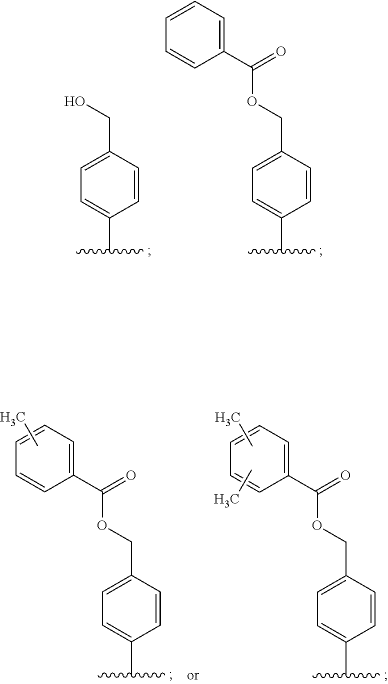

R² is hydrogen or —C₁₋₆-alkyl; and

R³ is hydrogen or —C₁₋₆-alkyl.

In some embodiments, R⁶ is hydrogen, methyl, —OH, or —CN.

In some embodiments, R² is hydrogen or methyl.

In some embodiments, R³ is hydrogen or methyl.

In some embodiments, R¹ is thienyl; phenyl; phenyl substituted with halogen or methyl; cyclohexyl; benzothiphene; —C(O)N(H)-phenyl;

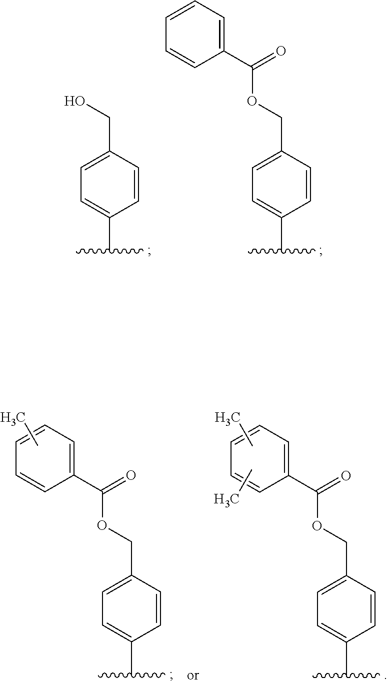

In some embodiments, the compound of Formula (V3) is a compound of Formula (V3a):

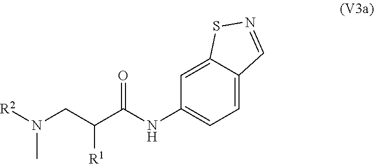

or a pharmaceutically acceptable salt thereof.

In some embodiments, R² is methyl.

In some embodiments, the compound is a compound of Table 2, or a pharmaceutically acceptable salt thereof.

In some embodiments, the compound of Formula (V) is a compound of Formula (V4):

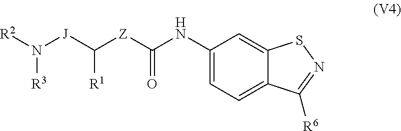

or a pharmaceutically acceptable salt thereof;

wherein

J is a bond, methylene, or ethylene;

Z is a bond, methylene, or ethylene;

R¹ is —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen; C₄₋₈-heteroaryl;

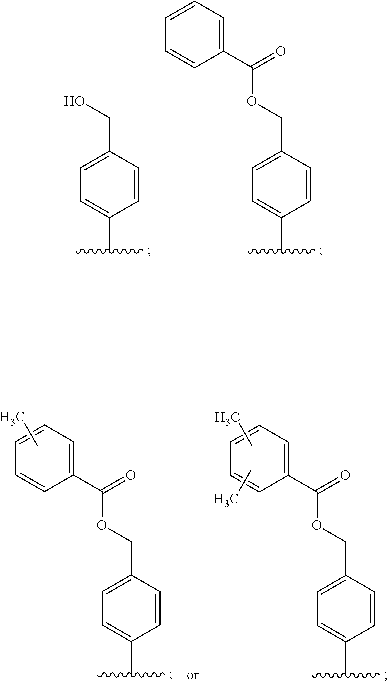

R² is hydrogen, —C₁₋₆-alkyl, —CH₂—(C₆₋₁₀-aryl), or —C(NH)NH₂; R³ is hydrogen or —C₁₋₆-alkyl;

or Z is CH and R³ and Z, together with the atoms to which they are attached, form a C₃₋₆-heterocycloalkyl; and

R⁶ is hydrogen, —C₁₋₆-alkyl, —OH, —O—(C₁₋₆-alkyl), —CN, or —C₁₋₆-haloalkyl.

In some embodiments, R⁶ is hydrogen, methyl, —OH, or —CN.

In some embodiments, R³ is hydrogen or methyl.

In some embodiments, R² is hydrogen, methyl, or benzyl.

In some embodiments, R³ is hydrogen and R² is —C(NH)NH₂.

In some embodiments, R³ is methyl and R² is methyl.

In some embodiments, R³ is hydrogen and R² is methyl.

In some embodiments, R¹ is hydrogen; —C₆₋₁₀-aryl; —C₆₋₁₀-aryl substituted with halogen; —C₃₋₁₀-cycloalkyl; or C₄₋₈-heteroaryl.

In some embodiments, R¹ is hydrogen; —C₆₋₁₀-aryl; —C₆₋₁₀-aryl substituted with halogen, —C₁₋₆-alkyl, or —(C₁₋₆-alkyl)-OH; —CH₂—(C₆₋₁₀-aryl); —CH₂—(C₆₋₁₀-aryl) substituted with halogen; or C₄₋₈-heteroaryl.

In some embodiments, R¹ is hydrogen; —C₆₋₁₀-aryl; —C₆₋₁₀-aryl substituted with halogen, —C₁₋₆-alkyl or —C₁₋₆-haloalkyl;

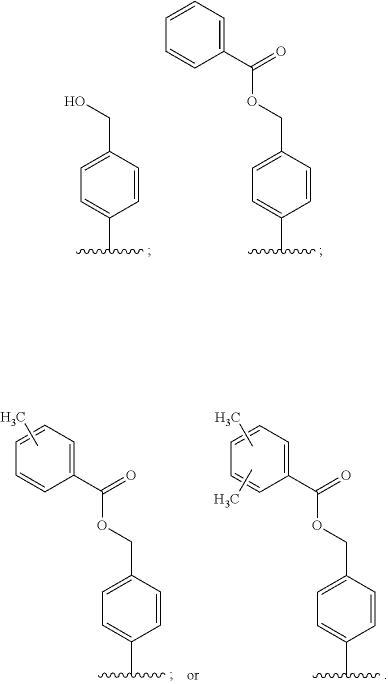

In some embodiments, the compound of Formula (V4) is a compound of Formula (V4a):

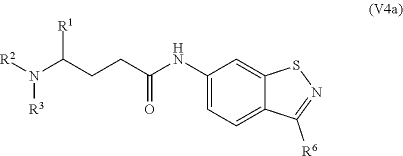

or a pharmaceutically acceptable salt thereof.

In some embodiments, the compound of Formula (V4) is a compound of Formula (V4b):

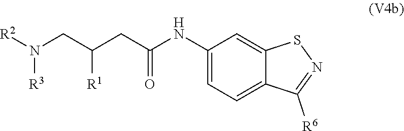

or a pharmaceutically acceptable salt thereof.

In some embodiments, the compound of Formula (V4) is a compound of Formula (V4c):

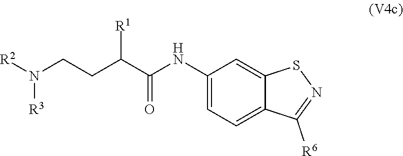

or a pharmaceutically acceptable salt thereof.

In some embodiments,

J is a bond;

Z is ethylene; and

R¹ is phenyl, phenyl substituted with halogen, cyclopropyl, thienyl, or cyclohexyl.

In some embodiments,

J is methylene;

Z is methylene; and

R¹ is phenyl, phenyl substituted with halogen, benzyl, benzyl substituted with halogen, or thienyl.

In some embodiments,

J is ethylene;

Z is a bond; and

R¹ is hydrogen; phenyl; phenyl substituted with halogen, methyl or fluoromethyl;

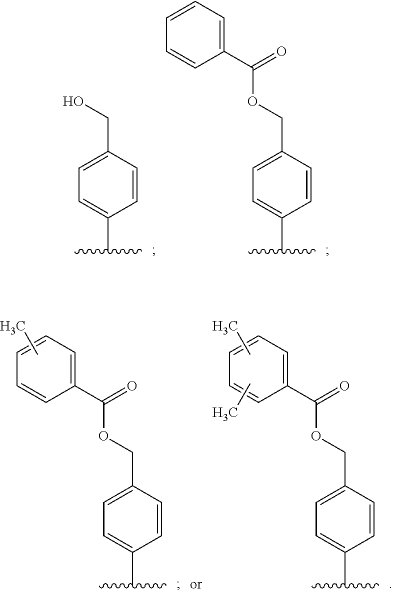

In some embodiments, Z is CH and R³ and Z, together with the atoms to which they are attached, form a C₃₋₆-heterocycloalkyl.

In some embodiments, the compound is a compound of Table 3, or a pharmaceutically acceptable salt thereof.

In some embodiments, the compound of Formula (V4) is a compound of Formula (V4d):

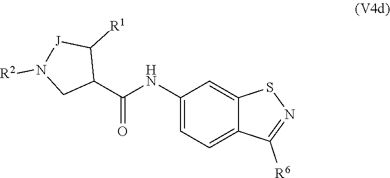

or a pharmaceutically acceptable salt thereof.

In some embodiments, Z is CH and R³ and Z, together with the atoms to which they are attached, form a pyrrolidinyl.

In some embodiments, the compound of Formula (V4) is a compound of Formula (V4e):

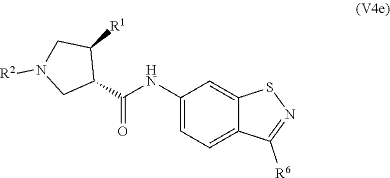

or a pharmaceutically acceptable salt thereof.

In some embodiments, RI is phenyl or phenyl substituted with halogen, methyl, ethyl, or —CH₂OH.

In another aspect, provided herein is a compound of Formula (VI):

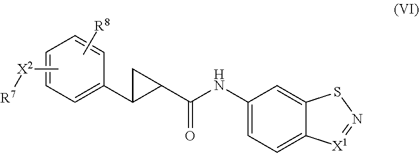

or a pharmaceutically acceptable salt thereof;

wherein

X¹ is C—R⁶ or N;

X² is —C(O)— or —SO₂—;

R⁷ is —OH; —NH₂; —O—(C₁₋₆-alkyl); —N(H)—(C₁₋₃-alkyl)-heteroaryl; —N(H)— heteroaryl; —N(H)—(C₁₋₃-alkyl)-(C₆₋₁₀-aryl)-(C₁₋₃-alkyl)-N(C₁₋₃-alkyl)₂; —N(H)—(C₆₋₁₀-aryl)-(C₁₋₃-alkyl)-N(C₁₋₃-alkyl)₂; —N(H)—(C₁₋₃-alkyl)-heteroalkyl; —N(H)—(C₁₋₃-alkyl)-N(C₁₋₃-alkyl)₂; heteroalkyl; -heteroalkyl-(C₆₋₁₀-aryl); —N(H)-heteroalkyl; heteroalkyl; —N(H)—(C₁₋₆-alkyl); —O—(C₁₋₃-alkyl)-heteroaryl; —O—(C₁₋₃-alkyl)-(C₆₋₁₀-aryl) substituted with —C₁₋₃-alkyl or C₁₋₃-haloalkyl; —O—(C₁₋₃-alkyl)-heteroalkyl; —O—(C₁₋₃-alkyl)-N(C₁₋₃-alkyl)₂; —O—(C₁₋₃-akyl)-(C₆₋₁₀-aryl); or —O—(C₁₋₆-alkyl);

R⁶ is hydrogen or —OH; and

R⁸ is hydrogen or halogen.

In some embodiments, X¹ is N.

In some embodiments, X¹ is C—R⁶.

In some embodiments, X² is —SO₂—.

In some embodiments, R⁷ is —OH; —NH₂; —OCH₃; —N(H)CH₂-pyridinyl; —N(H)-pyridinyl; —N(H)CH₂-phenyl-CH₂N(CH₃)₂; —N(H)-phenyl-CH₂N(CH₃)₂; —N(H)CH₂-piperidinyl; —N(H)CH₂-pyrrolidinyl; —N(H)CH₂CH₂N(CH₃)₂; morpholinyl; -piperazyinyl-phenyl; —N(H)-piperidinyl; diazepanyl; —N(H)CH₂CH₂-morpholinyl; —N(H)— butyl; —OCH₂-pyridinyl; —OCH₂-(methylphenyl); —OCH₂-piperidinyl; —OCH₂CH₂-(trifluoromethylphenyl); —OCH₂CH₂N(CH₃)₂; —OCHCH-phenyl; —O-pentanyl; or —N(H)-pyrimidinyl.

In some embodiments, R⁶ is H.

In some embodiments, R⁸ is halogen.

In some embodiments, the compound of Formula (VI) is a compound of (VII):

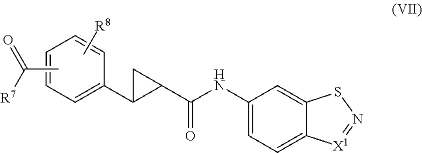

or a pharmaceutically acceptable salt thereof.

In some embodiments, the compound of Formula (VI) is a compound of Formula (VI2):

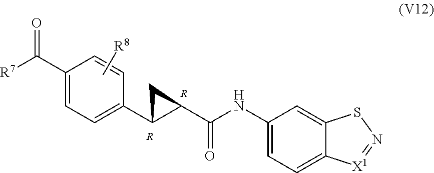

or a pharmaceutically acceptable salt thereof.

In some embodiments, the compound of Formula (VI) is a compound of Formula (VI3):

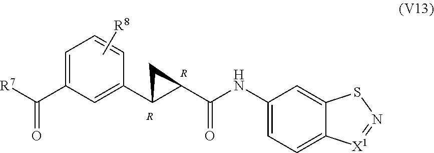

or a pharmaceutically acceptable salt thereof.

In some embodiments of the Formulae provided herein, the heteroaryl of R⁷ is pyridinyl or pyrimidinyl.

In some embodiments, the compound is a compound of Table 5, or a pharmaceutically acceptable salt thereof.

In some embodiments, the compound is a compound of Table 6, or a pharmaceutically acceptable salt thereof.

In some embodiments, the compound of Formula (V) is a compound of Formula (V5):

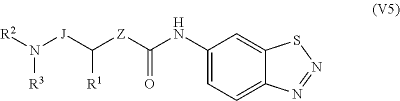

or a pharmaceutically acceptable salt thereof;

wherein

J is a bond, methylene or ethylene;

Z is a bond, methylene or ethylene;

or Z is CH and R³ and Z, together with the atoms to which they are attached, form a C₃₋₆-heterocycloalkyl;

R¹ is hydrogen; halogen; —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen, —C₁₋₆-alkyl, —C₁₋₆-haloalkyl, or —(C₁₋₆-alkyl)-OH; —C₄₋₁₀-heteroaryl;

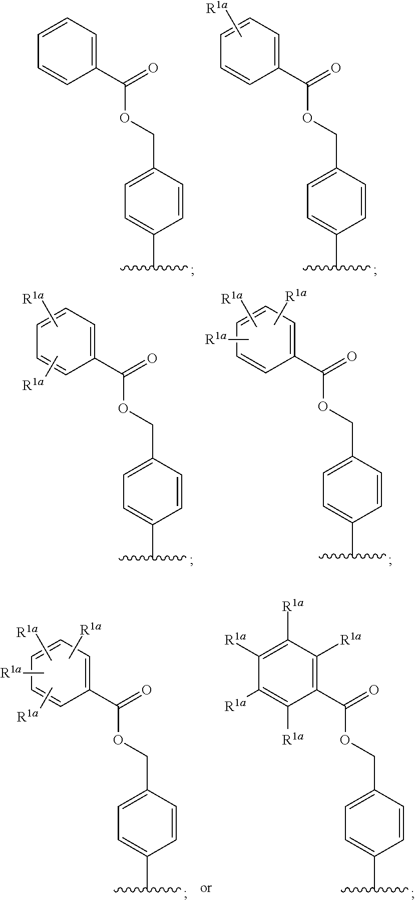

R^{1a} is, independently, halogen, —C₁₋₆-alkyl, or —C₁₋₆-haloalkyl;

R² is hydrogen, —C₁₋₆-alkyl, —(C₁₋₆-alkyl)N(C₁₋₆-alkyl) C₁₋₆-alkyl, or —C(NH)NH₂;

R³ is hydrogen or —C₁₋₆-alkyl;

or R² and R³, together with the atoms to which they are attached, form a C₂₋₆-heterocycloalkyl.

In some embodiments, R¹ is —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen, —C₁₋₆-alkyl, —C₁₋₆-haloalkyl, or —(C₁₋₆-alkyl)-OH; —C₄₋₁₀-heteroaryl;

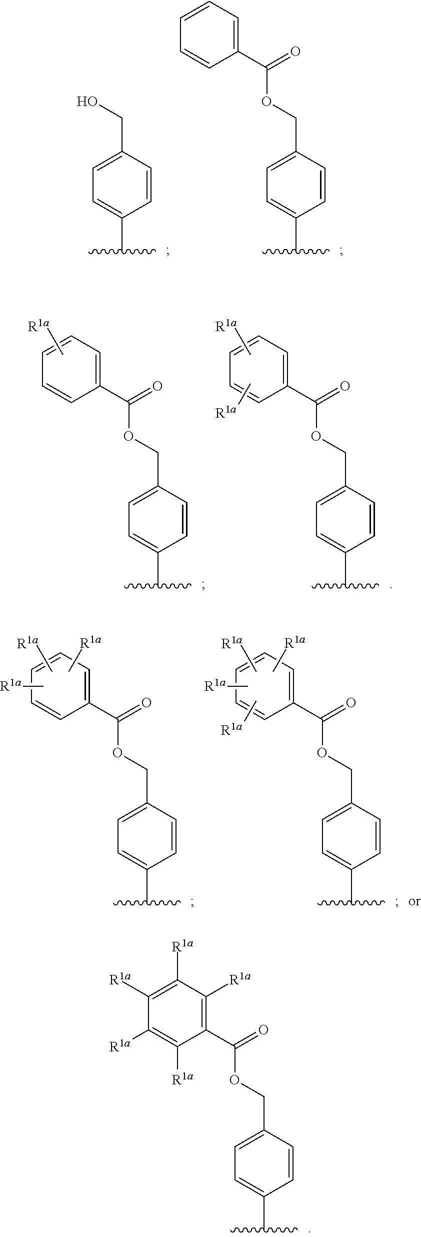

In some embodiments, R¹ is

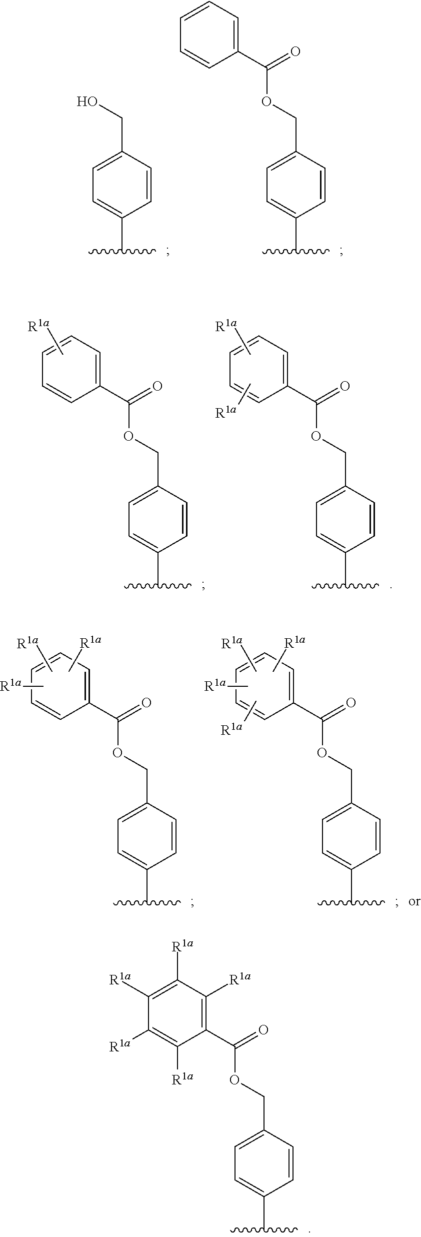

In some embodiments, R¹ is

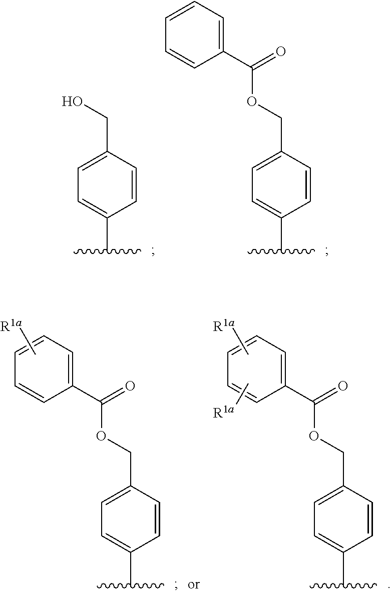

In some embodiments, R^{1a} is methyl, monohalo-methyl, dihalo-methyl, or trihalo-methyl.

In some embodiments, R¹ is

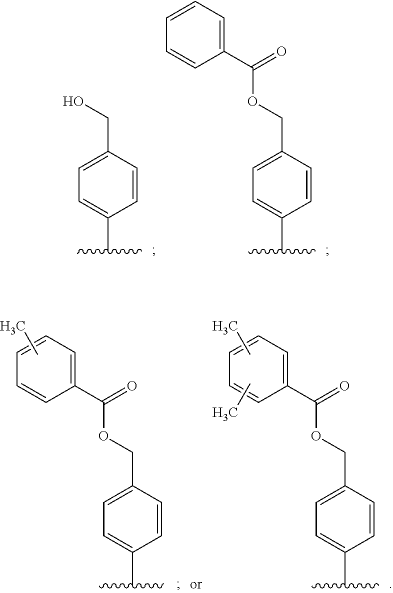

In some embodiments, R² and R³, together with the atoms to which they are attached, form an azetidinyl, pyrrolidinyl, piperidinyl, azepanyl, diazetidinyl, imidazolidinyl, piperazinyl, diazepanyl, oxazetidinyl, oxazolidinyl, morpholinyl, or oxazepanyl.

In some embodiments, Z is CH and R³ and Z, together with the atoms to which they are attached, form a C₃₋₆-heterocycloalkyl.

In some embodiments, the compound of Formula (V5) is a compound of Formula (V5a):

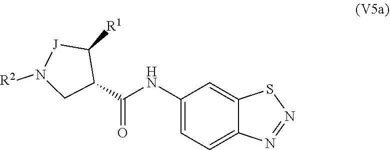

or a pharmaceutically acceptable salt thereof.

In some embodiments, Z is CH and R³ and Z, together with the atoms to which they are attached, form a pyrrolidinyl.

In some embodiments, the compound of Formula (V5) is a compound of Formula (V5b):

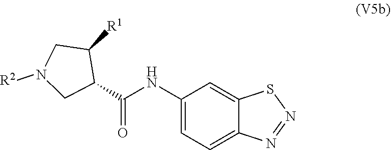

or a pharmaceutically acceptable salt thereof.

In some embodiments, R^{1a} is, independently, F, Cl, Br, —C₁₋₃-alkyl, or —C₁₋₃-haloalkyl.

In some embodiments, R^{1a} is, independently, F, Cl, methyl, monohalo-methyl, dihalo-methyl, or trihalo-methyl.

In some embodiments, R² is hydrogen, —C₁₋₃-alkyl, —(C₁₋₃-alkyl)N(C₁₋₃-alkyl) C₁₋₃-alkyl, —(C₁₋₃-alkyl)N(H) C₁₋₃-alkyl, —(C₁₋₃-alkyl)NH₂, or —C(NH)NH₂.

In some embodiments, R² is hydrogen, methyl, —(C₁₋₃-alkyl)N(CH₃) CH₃, —(C₁₋₃-alkyl)N(H) CH₃, —(C₁₋₃-alkyl)NH₂, or —C(NH)NH₂.

In some embodiments, R³ is hydrogen or —C₁₋₃-alkyl.

In some embodiments, R³ is hydrogen or methyl.

In some embodiments, R¹ is —C₃₋₆-cycloalkyl; phenyl; -phenyl substituted with halogen, —C₁₋₆-alkyl, —C₁₋₆-haloalkyl, or —(C₁₋₆-alkyl)-OH; benzyl; benzyl substituted with halogen, —C₁₋₆-alkyl, —C₁₋₆-haloalkyl, or —(C₁₋₆-alkyl)-OH; or —C₄₋₆-heteroaryl.

In some embodiments, R¹ is —C₃₋₆-cycloalkyl; phenyl; -phenyl substituted with halogen, —C₁₋₃-alkyl, —C₁₋₃-haloalkyl, or —(C₁₋₃-alkyl)-OH; benzyl; benzyl substituted with halogen, —C₁₋₃-alkyl, —C₁₋₃-haloalkyl, or —(C₁₋₃-alkyl)-OH; or —C₄₋₅-heteroaryl.

In some embodiments, J is ethylene and Z is a bond.

In some embodiments, J is a bond and Z is ethylene.

In some embodiments, J is methylene and Z is a bond.

In some embodiments, J is a bond and Z is methylene.

In some embodiments, J is methylene and Z is methylene.

In some embodiments, J is a bond and Z is a bond.

In some embodiments, the compound is a compound of Table 4, or a pharmaceutically acceptable salt thereof.

In some embodiments, the compound is a compound of Table 7, or a pharmaceutically acceptable salt thereof.

Compounds provided herein may be synthesized by general schemes 1-5 set forth below.

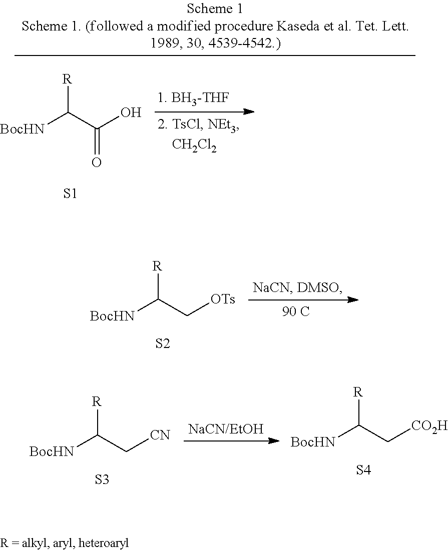

According to Scheme 1, the selected acid (S1) is reduced with an appropriate agent such as borane then activated as the tosylate to form the desired intermediate (S2). The tosylate (S2) is reacted with the sodium cyanide in DMSO to generate the nitrile (S3) directly which is then hydrolyzed with sodium hydroxide to form the one-carbon longer amino acid (S4). Following this scheme, alpha amino acids are transformed into beta amino acids and beta amino acids are turned into gamma, and gamma to delta in turn.

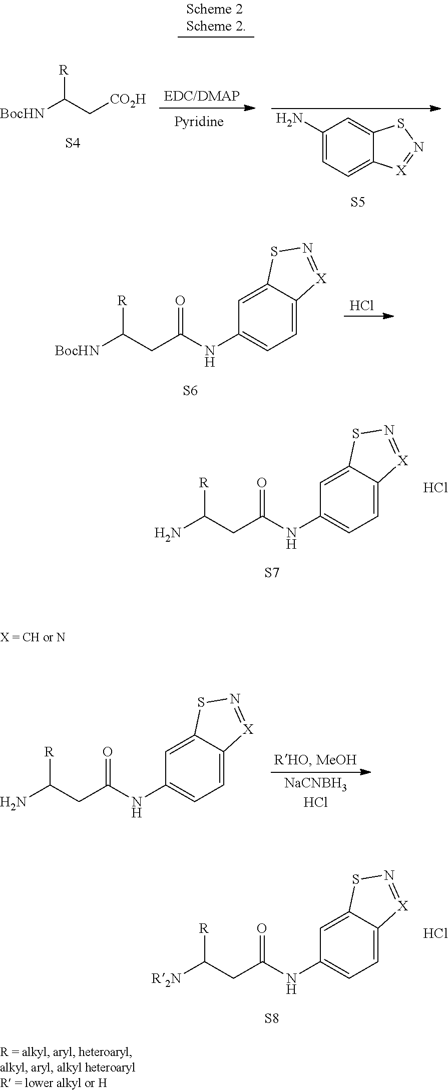

According to Scheme 2, the selected acid (S4) is activated with an appropriate agent such as EDC then coupled to a benzoisothiazole or a benzoisothiadiazole (S5) using standard coupling procedures to form the desired intermediate (S6). The carbamate (S6) is reacted with the HCl in methylene chloride to generate the amine (S7) directly. When alkyl groups are desired to be added, (S7) is subjected to reductive amination conditions to generate the N,N-disubstituted compounds of type (S8).

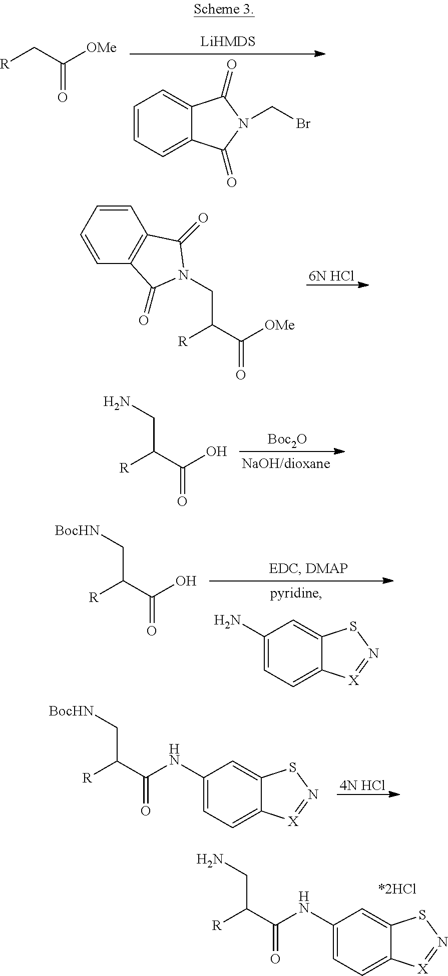

According to Scheme 3, the selected ester (S4) is alkylated with bromomethyl phthalimide and hydrolyzed to the free beta amino acid (S6). N-Boc protection and coupling with an appropriate agent such as EDC and amino-benzoisothiazole or amino-benzoisothiadiazole (S8) using standard coupling procedures to form the desired intermediate (S9). The protected carbamate (S9) is reacted with the HCl in methylene chloride to generate the amine (S10) directly. When alkyl groups are desired to be added, (S10) is subjected to reductive amination conditions to generate the N,N-disubstituted compounds.

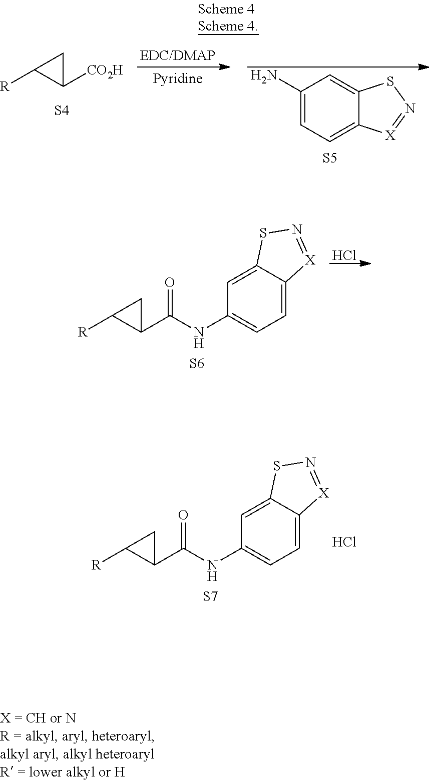

According to Scheme 4, the selected acid (S4) is activated with an appropriate agent such as EDC then coupled to an amino-benzoisothiazole or an amino-benzoisothiadiazole (S5) using standard coupling procedures to form the desired intermediate (S6). The compound (S6) is reacted with the HCl in methylene chloride to remove any protecting groups present and to generate the HCl salt of the compound (S7).

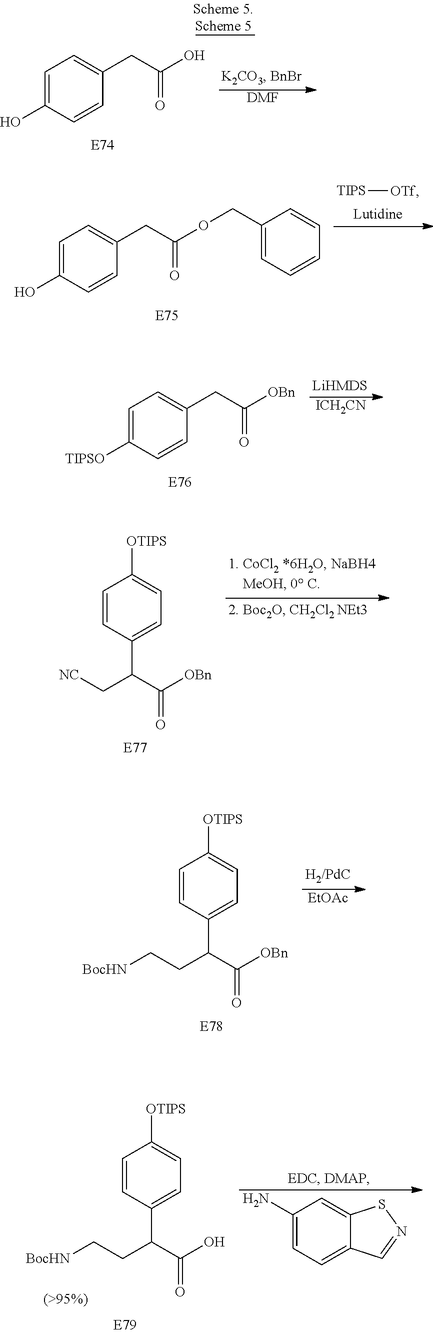

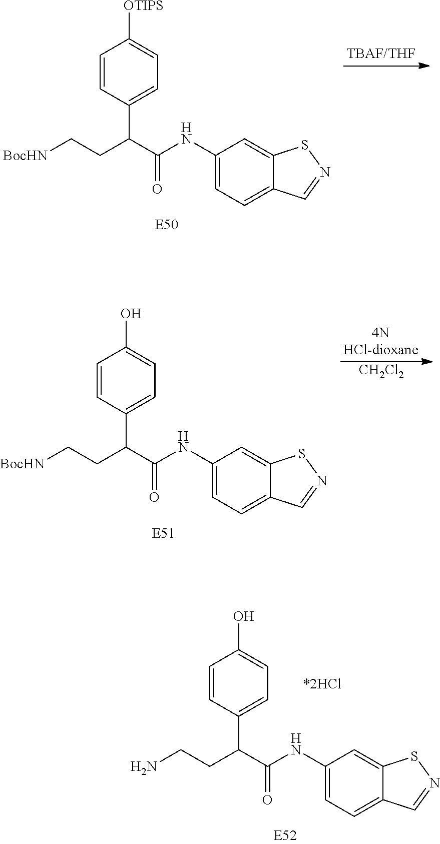

The abbreviations used in the synthetic schemes shown have the following meanings: Boc₂O means di-tert-butyl-dicarbonate, DMAP means dimethyl aminopyridine, DMSO means Dimethyl Sulfoxide, HATU means 2-(7-Aza-1H-benzotriazole-1-yl)-1,1,3,3-tetramethyluronium hexafluorophosphate, LDA means lithium diisopropyl amide, DMF is dimethylformamide, THF is tetrahydrofuran, and EDC means N-(3-dimethylaminopropyl)-N′-ethylcarbodiimide hydrochloride.

The compounds of the above Formulae and compositions including them have kinase inhibitory activity and are thus useful in influencing or inhibiting the action of kinases, and in treatment and/or prevention of diseases or conditions influenced by kinases. The above Formulae and compositions may be used to influence or inhibit the action of kinases either in a cell in vitro or in a cell in a living body in vivo. Specifically, in one embodiment, a method is provided of inhibiting the action of a kinase comprising applying to a medium such as an assay medium or contacting with a cell either in a cell in vitro or in a cell in a living body in vivo an effective inhibitory amount of a compound according to Formulae I or II or III. In a preferred embodiment, the kinase inhibited is a rho kinase. Compounds according to Formulae I or II or III are used in methods of inhibiting kinases in a cell, a tissue or an animal such as a human comprising administering to, or contacting with, the cell a therapeutically effective amount of one or more of these amino-benzoisothiazole and benzoisothiadiazole amide derivatives. The one or more of the amino-benzoisothiazole and benzoisothiadiazole amide are preferably administered in a pharmaceutically acceptable formulation, such as in or with a pharmaceutically acceptable carrier when the amino-benzoisothiazole and benzoisothiadiazole amide derivatives are administered to a cell or cells in a living organism or body. In another embodiment, the amino-benzoisothiazole and benzoisothiadiazole amide derivatives, according to Formulae I or II or III or IV, are used in methods for modulating the action of a kinase in a cell comprising contacting the cell with amount of one or more compounds according to Formulae I or II or III or IV effective to modulate the action of a kinase in a cell. The one or more of the amino-benzoisothiazole and benzoisothiadiazole amide derivatives, are preferably administered in a pharmaceutically acceptable formulation, such as in or with a pharmaceutically acceptable carrier when the amino-benzoisothiazole and benzoisothiadiazole amide are in contact a cell in a living organism or body.

Treatment or prevention of diseases or conditions for which the amino-benzoisothiazole and amino-benzoisothiadiazole amide derivatives may be useful includes any of the diseases or conditions associated with kinase activity or diseases or conditions affected by kinases. Examples of these types of diseases include retinal degradation such as AMD and DME, glaucoma, inflammatory diseases, hyperresponsiveness, skin diseases, cardiovascular diseases and cancers.

The benzoisothiazole and benzoisothiadiazole amide derivatives in some embodiments will be administered in conjunction with one or more additional therapeutic agents. Suitable additional therapeutic agents include, but are not limited to, rho kinase inhibitors, beta blockers, alpha-agonists, carbonic anhydrase inhibitors, prostaglandin-like compounds, miotic or cholinergic agents, or epinephrine compounds.

Beta blockers. These are thought to reduce the production of aqueous humor. Examples include levobunolol (BETAGAN™), timolol (BETIMOL™, TIMOPTIC™) betaxolol (BETOPTIC™) and metipranolol (OPTIPRANOLOL™).

Alpha-agonists. These are thought to reduce the production of aqueous humor and increase drainage. Examples include apraclonidine (IOPIDINE™) and brimonidine (ALPHAGAN™).

Carbonic anhydrase inhibitors. These also thought to reduce the production of aqueous humor. Examples include dorzolamide (TRUSOPT™) and brinzolamide (AZOPT™).

Prostaglandin-like compounds. These moieties are thought to increase the outflow of aqueous humor. Examples include latanoprost (XALATAN™), bimatoprost (LUMIGAN™), AR-102, tafluprost, and travoprost (TRAVATAN™).

Miotic or cholinergic agents. These are also thought to increase the outflow of aqueous humor. Examples include pilocarpine (ISOPTO CARPINE™, PILOPINE™) and carbachol (ISOPTO CARBACHOL™).

Epinephrine compounds. These compounds, such as dipivefrin (PROPINE), also are thought to increase the outflow of aqueous humor.

VEGF inhibitors. These compounds are thought to reduce the leakiness of blood vessels.

Steroids. These compounds reduce inflammation.

The additional therapeutic agent or agents can be administered simultaneously or sequentially with the compounds provided herein, including being present in an extended release formation. Sequential administration includes administration before or after the compounds provided herein. In some embodiments, the additional therapeutic agent or agents can be administered in the same composition as the compounds provided herein. In other embodiments, there can be an interval of time between administration of the additional therapeutic agent and the compounds provided herein.

In some embodiments, the administration of an additional therapeutic agent with a compound provided herein will enable lower doses of the other therapeutic agents to be administered for a longer period of time.

Thus, in one aspect provided herein are methods of treating a disease in a subject in need thereof, comprising administering to the subject an effective amount of a compound of any of the Formulae provided herein (e.g., Formula I, Formula II, Formula III, Formula IV, Formula V, Formula V1, Formula V2, Formula V3, Formula V3a, Formula V4, Formula V4a, Formula V4b, Formula V4c, Formula V4d, Formula V4e, Formula V5, Formula V5a, Formula V5b, Formula VI, Formula VII, or Formula V12). In some embodiments, the compound is a compound of Table 1, Table 2, Table 3, Table 4, Table 5, Table 6, or Table 7, or a pharmaceutically acceptable salt thereof.

In some embodiments, the disease comprises at least one of eye disease, bone disorder, obesity, heart disease, inflammatory disease, hepatic disease, renal disease, pancreatitis, cancer, myocardial infarct, gastric disturbance, hypertension, fertility control, disorders of hair growth, nasal congestion, neurogenic bladder disorder, gastrointestinal disorder, or dermatological disorder.

In some embodiments, the disease comprises an eye disease. In some embodiments, the disease is an eye disease.

In some embodiments, the eye disease comprises glaucoma or a neurodegenerative eye disease. In some embodiments, the eye disease is glaucoma, a neurodegenerative eye disease, dry eye, or ocular hypertension.

In another aspect, provided herein are methods of modulating kinase activity in a cell, comprising contacting the cell with a compound of any of the Formulae provided herein (e.g., Formula I, Formula II, Formula III, Formula IV, Formula V, Formula V1, Formula V2, Formula V3, Formula V3a, Formula V4, Formula V4a, Formula V4b, Formula V4c, Formula V4d, Formula V4e, Formula V5, Formula V5a, Formula V5b, Formula VI, Formula VII, or Formula VI2) in an amount effect to modulate kinase activity. In some embodiments, the compound is a compound of Table 1, Table 2, Table 3, Table 4, Table 5, Table 6, or Table 7, or a pharmaceutically acceptable salt thereof.

In some embodiments, the cell is in a subject.

In some embodiments, the subject is a human.

In another aspect, provided herein are methods of reducing intraocular pressure in a subject in need thereof, comprising contacting the subject with an effective amount of a compound of any of the Formulae provided herein (e.g., Formula I, Formula II, Formula III, Formula IV, Formula V, Formula V1, Formula V2, Formula V3, Formula V3a, Formula V4, Formula V4a, Formula V4b, Formula V4c, Formula V4d, Formula V4e, Formula V5, Formula V5a, Formula V5b, Formula VI, Formula VII, or Formula VI2). In some embodiments, the compound is a compound of Table 1, Table 2, Table 3, Table 4, Table 5, Table 6, or Table 7, or a pharmaceutically acceptable salt thereof.

Compositions including these amino-benzoisothiazole and benzoisothiadiazole amide derivatives of the Formulae provided herein may be obtained in the form of various salts or solvates. As the salts, physiologically acceptable salts or salts available as raw materials are used.

Compositions may include one or more of the isoforms of the Formulae provided herein when present. When racemates exists, each enantiomer or diastereomer may be separately used, or they may be combined in any proportion. Where tautomers exist all possible tautomers are specifically contemplated.

Pharmaceutical compositions for use in accordance with the present disclosure may be formulated in a conventional manner using one or more physiologically acceptable carriers or excipients. Thus, the compounds of the Formulae provided herein and their physiologically acceptable salts and solvates may be formulated for administration by, for example, solid dosing, eyedrop, in a topical oil-based formulation, injection, inhalation (either through the mouth or the nose), implants, or oral, buccal, parenteral or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

Thus, compositions are provided herein comprising a compound of any of the Formulae provided herein (e.g., Formula I, Formula II, Formula III, Formula IV, Formula V, Formula V1, Formula V2, Formula V3, Formula V3a, Formula V4, Formula V4a, Formula V4b, Formula V4c, Formula V4d, Formula V4e, Formula V5, Formula V5a, Formula V5b, Formula VI, Formula VII, or Formula VI2). In some embodiments, the compound is a compound of Table 1, Table 2, Table 3, Table 4, Table 5, Table 6, or Table 7, or a pharmaceutically acceptable salt thereof.

Also provided herein are pharmaceutical compositions comprising a compound of any of the Formulae provided herein (e.g., Formula I, Formula II, Formula III, Formula IV, Formula V, Formula V1, Formula V2, Formula V3, Formula V3a, Formula V4, Formula V4a, Formula V4b, Formula V4c, Formula V4d, Formula V4e, Formula V5, Formula V5a, Formula V5b, Formula VI, Formula VII, or Formula V12) and a pharmaceutically acceptable carrier. In some embodiments, the compound is a compound of Table 1, Table 2, Table 3, Table 4, Table 5, Table 6, or Table 7, or a pharmaceutically acceptable salt thereof.

In some embodiments of the pharmaceutical compositions, the carrier is saline buffered to a pH of about 5.5 to about 6.5.

In some embodiments of the pharmaceutical compositions, the carrier is saline buffered to a pH of about 4.5 to about 5.5.

In some embodiments of the pharmaceutical compositions, the carrier is saline buffered to a pH of about 4.9 to about 5.1.

Compositions provided herein may comprise a safe and effective amount of the subject compounds, and a pharmaceutically-acceptable carrier. As used herein, “safe and effective amount” means an amount of a compound sufficient to significantly induce a positive modification in the condition to be treated, but low enough to avoid serious side effects (at a reasonable benefit/risk ratio), within the scope of sound medical judgment. A safe and effective amount of a compound will vary with the particular condition being treated, the age and physical condition of the patient being treated, the severity of the condition, the duration of treatment, the nature of concurrent therapy, the particular pharmaceutically-acceptable carrier utilized, and like factors within the knowledge and expertise of the attending physician.

The route by which the compounds provided herein (component A) will be administered and the form of the composition will dictate the type of carrier (component B) to be used. The composition may be in a variety of forms, suitable, for example, for systemic administration (e.g., oral, rectal, nasal, sublingual, buccal, implants, or parenteral) or topical administration (e.g., local application on the skin, ocular, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, q) biodegradable polymers, r) plasticizers, combinations thereof, and others. All carriers are optional in the systemic compositions.

Although the amounts of components A and B in the systemic compositions will vary depending on the type of systemic composition prepared, the specific derivative selected for component A and the ingredients of component B, in general, system compositions comprise 0.01% to 50% of component A and 50 to 99.99% of component B.

Compositions for parenteral administration typically comprise A) 0.1 to 10% of the compounds provided herein and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof.

Capsules (including implants, time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this disclosure. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include implanted, sublingual, buccal and nasal dosage forms. Such compositions typically comprise one or more of soluble or biodegradable filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants. Implanted formulations typically include q) biodegradable polymers and optionally, r) plasticizers.

In one embodiment of the disclosure, the compounds provided herein are topically administered. Topical compositions that can be applied locally to the eye may be in any form known in the art, non-limiting examples of which include solids, gellable drops, sprays, ointments, or a sustained or non-sustained release unit placed in the conjunctival cul-du-sac of the eye, in the intracameral space, in the aqueous humor, in the vitreous humor, or another appropriate location.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions comprise: component A, the compounds described above, and component B, a carrier. The carrier of the topical composition preferably aids penetration of the compounds into the eye. Component B may further comprise one or more optional components.

The dosage range of the compound for systemic administration is from about 0.01 to about 1000 μg/kg body weight, preferably from about 0.1 to about 100 μg/kg per body weight, most preferably form about 1 to about 50 μg/kg body weight per day. While these dosages are based upon a daily administration rate, weekly or monthly accumulated dosages may also be used to calculate the clinical requirements.

Dosages may be varied based on the patient being treated, the condition being treated, the severity of the condition being treated, the route of administration, etc. to achieve the desired effect.

The compounds provided herein are useful in decreasing intraocular pressure. Thus, these compounds are useful in the treatment of glaucoma. The preferred route of administration for treating glaucoma is topically.

The exact amounts of each component in the topical composition depend on various factors. The amount of component A added to the topical composition is dependent on the IC₅₀ of component A, typically expressed in nanomolar (nM) units. For example, if the IC₅₀ of the medicament is 1 nM, the amount of component A will be from about 0.001 to about 0.3%. If the IC₅₀ of the medicament is 10 nM, the amount of component A) will be from about 0.01 to about 1%. If the IC₅₀ of the medicament is 100 nM, the amount of component A will be from about 0.1 to about 10%. If the IC₅₀ of the medicament is 1000 nM, the amount of component A will be 1 to 100%, preferably 5% to 50%. If the amount of component A is outside the ranges specified above (i.e., lower), efficacy of the treatment may be reduced. One skilled in the art understands how to calculate and understand an IC₅₀. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this disclosure are described in the following references: Modem Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B may comprise a single ingredient or a combination of two or more ingredients. In the topical compositions, component B comprises a topical carrier.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Component A may be included in kits comprising component A, a systemic or topical composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans). Component A may also be included in articles of manufacture for use as described herein for compounds provided herein.

Thus, provided herein are kits comprising a compound of any of the Formulae provided herein, and instructions for use thereof. Also provided herein are kits comprising a pharmaceutical composition provided herein, and instructions for use thereof.

Also provided herein are articles of manufacture comprising a compound of any of the Formulae provided herein. Also provided herein are articles of manufacture comprising a pharmaceutical composition provided herein, and instructions for use thereof.

The following illustrative examples are to be considered non-limiting.

Specific procedures for the preparation of amino-benzoisothiazole and benzoisothiadiazole amide are described in the following examples.

All temperatures are in degrees Centigrade. Reagents and starting materials were purchased from commercial sources or prepared following published literature procedures.

Unless otherwise noted, HPLC purification, when appropriate, was performed by redissolving the compound in a small volume of DMSO and filtering through a 0.45 micron (nylon disc) syringe filter. The solution was then purified using, for example, a 50 mm Varian Dynamax HPLC 21.4 mm Microsorb Guard-8 C₈ column. A typical initial eluting mixture of 40-80% MeOH:H₂O was selected as appropriate for the target compound. This initial gradient was maintained for 0.5 minutes then increased to 100% MeOH: 0% H₂O over 5 minutes. 100% MeOH was maintained for 2 more minutes before re-equilibration back to the initial starting gradient. A typical total run time was 8 minutes. The resulting fractions were analyzed, combined as appropriate, and then evaporated to provide purified material.

Proton magnetic resonance (¹H NMR) spectra were recorded on either a Varian INOVA 600 MHz (¹H) NMR spectrometer, Varian INOVA 500 MHz (H) NMR spectrometer, Varian Mercury 300 MHz (H) NMR spectrometer, or a Varian Mercury 200 MHz (H) NMR spectrometer. All spectra have been determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

Analytical LCMS spectra were obtained using a Waters ZQ MS ESI instrument with an Alliance 2695 HPLC and a 2487 dual wavelength UV detector. Spectra were analyzed at 254 and 230 nm. Samples were passed through a Waters Symmetry C_{18 4.6}×75 mm 3.5 column with or without a guard column (3.9×20 mm 5). Gradients were run with mobile phase A: 0.1% formic acid in H₂O and mobile phase B: ACN with a flow rate of 0.8 mL/min. Gradient A is illustrative of a gradient used for analytical LCMS:

Gradient A

| Time | A % | B % |
|---|---|---|
| 0.00 | 80.0 | 20.0 |
| 1.00 | 80.0 | 20.0 |
| 6.00 | 25.0 | 75.0 |
| 7.00 | 5.0 | 95.0 |
| 8.00 | 5.0 | 95.0 |
| 9.00 | 80.0 | 20.0 |
| 12.00 | 80.0 | 20.0 |

The settings for the MS probe were a cone voltage at 38 mV and a desolvation temperature at 250° C. Any variations in these methods are noted below.

The following preparations illustrate procedures for the preparation of intermediates and methods for the preparation of amino-benzoisothiazole and benzoisothiadiazole amide compounds provided herein.

### EXAMPLES

### Example 1. Preparation of tert-butyl 2-hydroxy-1-(thiophen-3-yl)ethylcarbamate (E1)

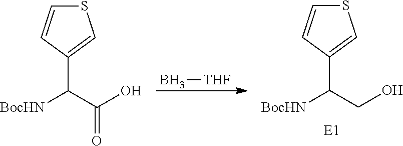

To (±)-2-(tert-butoxycarbonylamino)-2-(thiophen-3-yl)acetic acid in THF at 0° C. was added BH₃-THF dropwise. The solution was allowed to warm to room temperature and stirred for an additional 2 hours. The solution was cooled to 0° C., quenched with AcOH (10%)/MeOH and evaporated. Column chromatography (SiO₂, EtOAc) gave pure tert-butyl 2-hydroxy-1-(thiophen-3-yl)ethylcarbamate (E1).

### Example 2. Preparation of 2-(tert-butoxycarbonylamino)-2-(thiophen-3-yl)ethyl 4-methylbenzenesulfonate (E2)

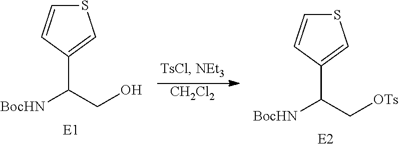

To tert-butyl 2-hydroxy-1-(thiophen-3-yl)ethylcarbamate (E1) in CH₂Cl₂ was added NEt₃, DMAP, and TsCl. The solution was stirred at room temperature for 3 hours and then poured into NH₄Cl (sat) and extracted with EtOAc, dried (Na₂SO₄), filtered, and evaporated. Column chromatography (SiO₂, 30% EtOAc/Hexanes) gave pure 2-(tert-butoxycarbonylamino)-2-(thiophen-3-yl)ethyl 4-methylbenzenesulfonate (E2).

### Example 3. Preparation of tert-butyl 2-cyano-1-(thiophen-3-yl)ethylcarbamate (E3)

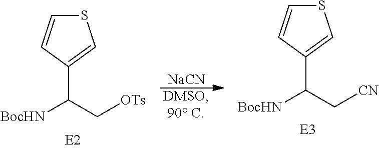

To 2-(tert-butoxycarbonylamino)-2-(thiophen-3-yl)ethyl 4-methylbenzenesulfonate (E2) in DMSO was added NaCN, and the solution was heated to 90° C. for 2 hours. The reaction was cooled, poured into NaCl (sat), and extracted with EtOAc. The organics were dried (Na₂SO₄), filtered, and evaporated. Column chromatography (SiO₂, 25% EtOAc/Hexanes) gave pure tert-butyl 2-cyano-1-(thiophen-3-yl)ethylcarbamate (E3).

### Example 4. Preparation of 3-(tert-butyoxcarbonylamino)-3-(thiophen-3-yl)propanoic acid (E4)

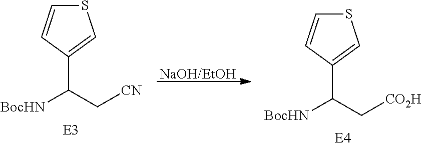

To tert-butyl 2-cyano-1-(thiophen-3-yl)ethylcarbamate (E3) in EtOH was added NaOH (2M), and the solution was heated to 90° C. for 4 hours. The reaction was cooled, acidified with HCl, and extracted with EtOAc. The organics were dried (Na₂SO₄) and evaporated to give pure 3-(tert-butyoxcarbonylamino)-3-(thiophen-3-yl)propanoic acid (E4).

### Example 5. Preparation of tert-butyl 3-(benzoisothiazol-6-ylamino)-3-oxo-1-(thiophen-3-yl)propylcarbamate (E5)

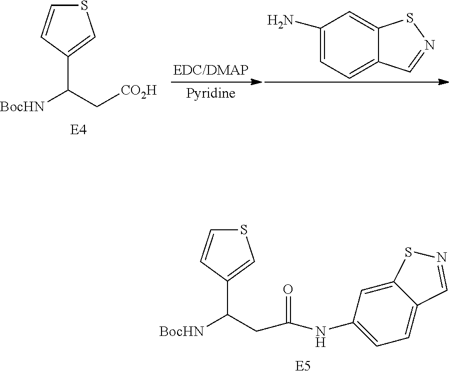 [SMILES: CC(C)(C)OC(=O)NC(CC(=O)O)c1ccsc1 ; Nc1ccc2cnsc2c1 ; CC(C)(C)OC(=O)NC(CC(=O)Nc1ccc2cnsc2c1)c1ccsc1]

To 3-(tert-butyoxcarbonylamino)-3-(thiophen-3-yl)propanoic acid (E4) in pyridine were added EDC, DMAP, and 6-aminobenzoisothiazole (also known as benzo[d]isothiazol-6-amine). The solution was stirred for 10 hours at room temperature. The mixture was poured into NaHCO₃₍ₛₐₜ₎ and extracted with EtOAc, dried (Na₂SO₄), filtered, and evaporated. Column chromatography (SiO₂, 5% MeOH/CH₂C₂) gave pure tert-butyl 3-(benzoisothiazol-6-ylamino)-3-oxo-1-(thiophen-3-yl)propylcarbamate (E5).

### Example 6. Preparation of 3-amino-N-(benzoisothiazol-6-yl)-3-(thiophen-3-yl)propanamide dihydrochloride (E6)

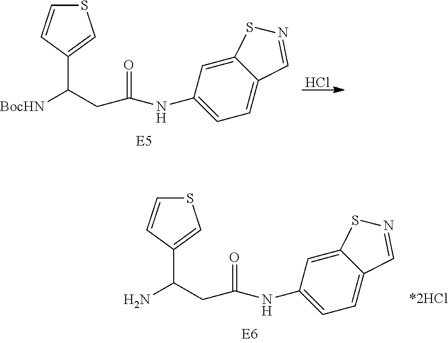 [SMILES: CC(C)(C)OC(=O)NC(CC(=O)Nc1ccc2cnsc2c1)c1ccsc1 ; Cl ; NC(CC(=O)Nc1ccc2cnsc2c1)c1ccsc1]

To tert-butyl 3-(benzoisothiazol-6-ylamino)-3-oxo-1-(thiophen-3-yl)propylcarbamate (E5) in CH₂Cl₂ was added HCl (4N in dioxane), and the solution was stirred for 8 hours. The solvents were evaporated to give 3-amino-N-(benzoisothiazol-6-yl)-3-(thiophen-3-yl)propanamide dihydrochloride (E6).

Using commercially available compounds and largely the procedures set forth in Examples 1-6 and substituting the appropriate starting materials, the following compounds of Table 1 can be made.

Thus, in some embodiments provided herein are compounds of the following Formula:

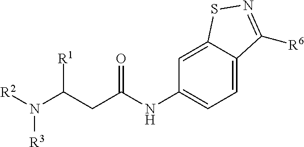

or a pharmaceutically acceptable salt thereof,

wherein

R¹ is (S)—C₆H₅, (R)—C₆H₅, (+)-o-chloro-C₆H₄, (±)-p-chloro-C₆H₄, (±)-p-hydroxymethyl-C₆H₄, (±)-p-fluoro-C₆H₄, (S)-3-thienyl, (R)-3-thienyl, (S)—CH₂-2-thienyl, (S)-2-thienyl, (R)-2-thienyl, 3-furyl, 2-furyl, 3,5-difluoroC₆H₃, CH₃, (S)-3-pyridyl, 4-pyridyl, benzyl, cyclohexyl, cyclopropyl, methyl cyclohexyl, 4-fluorobenzyl, 2-thiazole, 2-oxazole, or 3-piperdyl;

R² is H or Me;

R³ is H or Me; and

R⁶ is H or OH.

TABLE 1

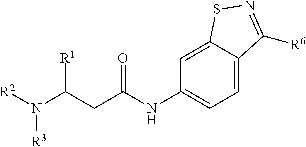

| Example | R⁶ | R¹ | R² | R³ |
|---|---|---|---|---|
| 7 | H | (S)-C₆H₅ | H | H |
| 8 | H | (R)-C₆H₅ | H | H |
| 9 | OH | (S)-C₆H₅ | H | H |
| 10 | OH | (R)-C₆H₅ | H | H |
| 11 | OH | (S)-C₆H₅ | Me | Me |
| 12 | H | (S)-C₆H₅ | Me | H |
| 13 | H | (±)-o-chloro-C₆H₄ | H | H |
| 14 | H | (±)-p-chloro-C₆H₄ | H | H |
| 15 | H | (±)-p-hydroxymethyl-C₆H₄ | H | H |
| 16 | OH | (±)-p-fluoro-C₆H₄ | H | H |
| 17 | H | (±)-p-fluoro-C₆H₄ | Me | H |
| 18 | H | (S)-3-thienyl | H | H |
| 19 | OH | (S)-3-thienyl | H | H |
| 20 | H | (S)-3-thienyl | Me | Me |
| 21 | OH | (S)-3-thienyl | Me | Me |
| 22 | H | (R)-3-thienyl | Me | Me |
| 23 | OH | (S)-CH₂-2-thienyl | Me | Me |
| 24 | OH | (S)-3-thienyl | Me | H |
| 25 | H | (S)-2-thienyl | Me | Me |
| 26 | H | (R)-2-thienyl | Me | Me |
| 27 | H | 3-fury! | H | H |
| 28 | OH | 2-fury! | Me | Me |
| 29 | OH | 3,5-difluoroC₆H₃ | Me | H |
| 30 | H | m-CH₃ | H | H |
| 31 | H | (S)-3-pyridyl | H | H |
| 32 | OH | 4-pyridyl | Me | Me |
| 33 | H | Benzyl | H | H |
| 34 | H | Cyclohexyl | Me | Me |
| 35 | H | Cyclopropyl | H | H |
| 36 | OH | Methyl-cyclohexyl | Me | H |
| 37 | H | 4-fluorobenzyl | H | H |
| 38 | H | 2-thiazole | Me | Me |
| 39 | OH | 2-oxazole | H | Me |
| 40 | H | 3-piperdyl | Me | Me |

### Example 7. Preparation of methyl 2-(thiophen-3-yl)acetate_(E41)

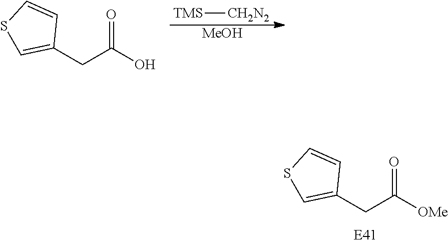

To 2-(thiophen-3-yl)acetic acid in MeOH at 0° C. was added TMS-CH₂N₂. The solution was stirred for 3 hours then quenched with a few drops of AcOH. The solvents were evaporated. Column chromatography (SiO₂, 3-15% EtOAc/Hex) gave pure methyl 2-(thiophen-3-yl)acetate (E41).

### Example 42. Preparation of methyl 3-(dimethylamino)-2-(thiophen-3-yl)propanoate (E42)

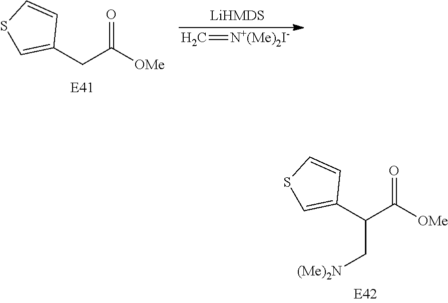

To methyl 2-(thiophen-3-yl)acetate (E41) in THF cooled to −78° C. was added LiHMDS, and the solution stirred at −78° C. for 30 minutes. Then N,N-dimethylmethyleneiminium iodide (Eschenmoser's salt) was added directly and the solution was allowed to warm to 0° C. The mixture was poured into NaHCO₃ (sat. aq.), extracted with EtOAc, dried over Na₂SO₄, filtered, and the solvent evaporated. Column chromatography (SiO₂, 5% MeOH/CH₂Cl₂) gave pure methyl 3-(dimethylamino)-2-(thiophen-3-yl)propanoate (E42).

### Example 43. Preparation of 3-(dimethylamino)-2-(thiophen-3-yl)propanoic acid (E43)

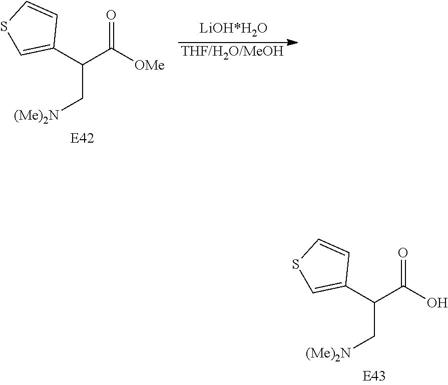

To methyl 3-(dimethylamino)-2-(thiophen-3-yl)propanoate (E42) in THF/H₂O/MeOH was added LiOH.H₂O, and the solution was stirred for 12 hours. AcOH was added and the solvents were evaporated. Column chromatography (SiO₂, 10-15% 2M NH₃ in MeOH/EtOH) gave pure 3-(dimethylamino)-2-(thiophen-3-yl)propanoic acid (E43).

### Example 44. Preparation of 3-(dimethylamino)-N-(benzoisothiazol-6-yl)-2-(thiophen-3-yl)propanamide dihydrochloride (E44)

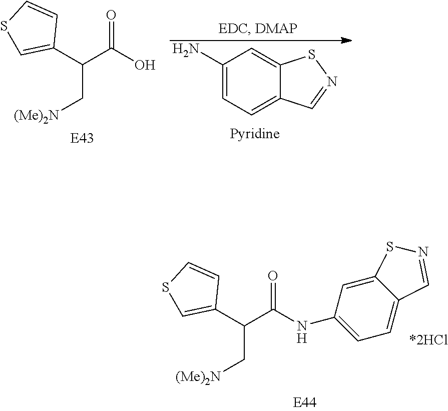

To 3-(dimethylamino)-2-(thiophen-3-yl)propanoic acid (E43) in pyridine was added EDC, DMAP, and 6-amino-benzoisothiazole. The solution was stirred overnight at room temperature. The mixture was poured into NaHCO₃₍ₛₐₜ₎ and extracted with EtOAc. The organics were dried (Na₂SO₄), filtered, and evaporated. Column chromatography (SiO₂, 5-20% MeOH/CH₂Cl₂) gave pure 3-(dimethylamino)-N-(benzoisothiazol-6-yl)-2-(thiophen-3-yl)propanamide. The pure compound was taken up in CH₂Cl₂ and HCl was added. The solvents were evaporated to give pure 3-(dimethylamino)-N-(benzoisothiazol-6-yl)-2-(thiophen-3-yl)propanamide dihydrochloride (E44).

### Example 45. Preparation of methyl 3-(1,3-dioxoisoindolin-2-yl)-2-(thiophen-3-yl)propanoate (E45)

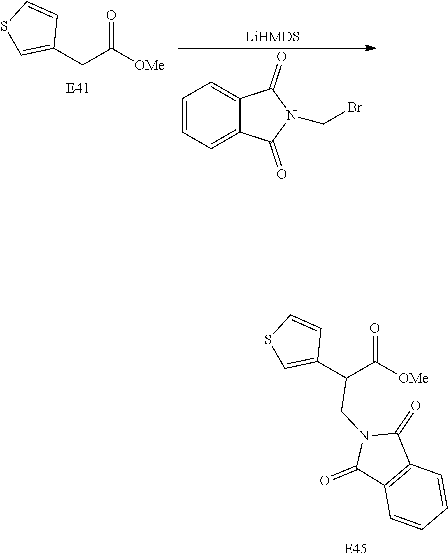 [SMILES: COC(=O)Cc1ccsc1 ; O=C1c2ccccc2C(=O)N1CBr ; COC(=O)C(CN1C(=O)c2ccccc2C1=O)c1ccsc1]

To pure methyl 2-(thiophen-3-yl)acetate (E41) in THF cooled to −78° C. was added LiHMDS, and the solution stirred at −78° C. for 30 min. Then N-(bromomethyl)phthalimide was added directly, and the solution was allowed to warm to 0° C. The mixture was poured into NaHCO₃₍ₛₐₜ₎, extracted with EtOAc, dried (Na₂SO₄), filtered, and evaporated. Column chromatography (SiO₂, 0-40% EtOAc/Hex) gave pure methyl 3-(1,3-dioxoisoindolin-2-yl)-2-(thiophen-3-yl)propanoate (E45).

### Example 46. Preparation of 3-amino-2-(thiophen-3-yl)propanoic acid hydrochloride (E46)

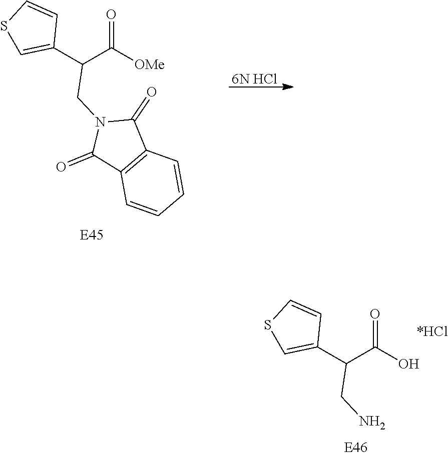

To methyl 3-(1,3-dioxoisoindolin-2-yl)-2-(thiophen-3-yl)propanoate (E45) was added 6 N HCl, and the solution was refluxed for 4 hours. The solvents were evaporated to give 3-amino-2-(thiophen-3-yl)propanoic acid (E46).

### Example 47. Preparation of 3-(tert-butoxycarbonylamino)-2-(thiophen-3-yl)propanoic acid (E47)

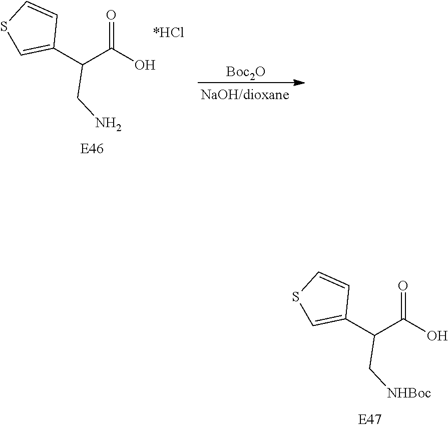 [SMILES: "Boc2O" ; NCC(C(=O)O)c1ccsc1 ; CC(C)(C)OC(=O)NCC(C(=O)O)c1ccsc1]

To Boc₂O in dioxane at 0° C. was added a cooled solution (0° C.) of 3-amino-2-(thiophen-3-yl)propanoic acid hydrochloride (E46) in 1 N NaOH. The solution was stirred at 0° C. for 30 min, then at room temperature for 4 hours. The mixture was acidified with HCl and extracted with EtOAc and saturated aqueous NH₄Cl. The organics were dried (Na₂SO₄), filtered, and evaporated to give pure 3-(tert-butoxycarbonylamino)-2-(thiophen-3-yl)propanoic acid (E47).

### Example 48. Preparation of tert-butyl 3-(benzoisothiazol-6-ylamino)-3-oxo-2-(thiophen-3-yl)propylcarbamate (E48)

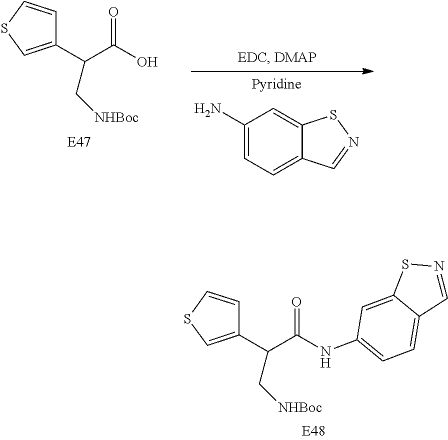 [SMILES: CC(C)(C)OC(=O)NCC(C(=O)O)c1ccsc1 ; Nc1ccc2cnsc2c1 ; CC(C)(C)OC(=O)NCC(C(=O)Nc1ccc2cnsc2c1)c1ccsc1]

To 3-(tert-butoxycarbonylamino)-2-(thiophen-3-yl)propanoic acid (E47) in pyridine was added EDC, DMAP, and 6-aminobenzoisothiazole. The solution was stirred overnight at room temperature. The mixture was poured into NaHCO₃ (sat) and extracted with EtOAc. The organics were dried (Na₂SO₄), filtered, and evaporated. Column chromatography (SiO₂, 3% MeOH/CH₂Cl₂) gave pure tert-butyl 3-(benzoisothiazol-6-ylamino)-3-oxo-2-(thiophen-3-yl) propylcarbamate (E48).

### Example 49. Preparation of 3-amino-N-(benzoisothiazol-6-yl)-2-(thiophen-3-yl)propanamide dihydrochloride (E49)

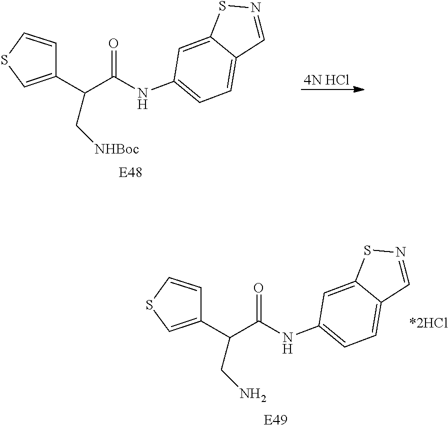

To tert-butyl 3-(benzoisothiazol-6-ylamino)-3-oxo-2-(thiophen-3-yl)propylcarbamate (E48) in CH₂Cl₂ was added HCl (4N in dioxane), and the solution was stirred for 8-10 hours. The solvents were evaporated to give pure 3-amino-N-(benzoisothiazol-6-yl)-2-(thiophen-3-yl)propanamide dihydrochloride (E49).

Using commercially available compounds and largely the procedures set forth in Examples 41-49 and substituting the appropriate starting materials, the compounds 50-72 of Table 2 can be made.

Thus, in some embodiments, provided herein are compounds of the following Formula:

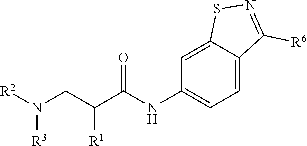

or a pharmaceutically acceptable salt thereof

wherein

R¹ is (±)-3-thienyl, C₆H₅, (R)—C₆H₅, (S)—C₆H₅, (±)-2-thienyl, p-fluoro-C₆H₄, 4-chloro-C₆H₄, (S)-4-chloro-C₆H4, 4-methyl-C₆H₄, (S)-4-CH₃—C₆H₄, cyclohexyl, 3-benzo[b]thiophene,

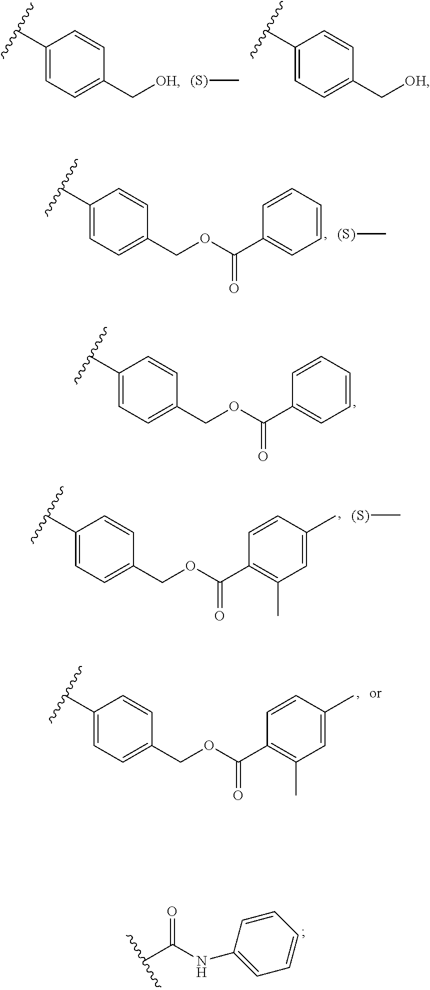

R² is H or Me;

R³ is H or Me; and

R⁶ is H, OH, CN, OMe, CHF—CH₃, CH₂F, or CH₃.

TABLE 2

| Example | R⁶ | R¹ | R² | R³ |
|---|---|---|---|---|
| 50 | OH | (±)-3-thienyl | Me | Me |
| 51 | H | (±)-3-thienyl | H | H |
| 52 | H | C₆H₅ | H | H |
| 53 | H | C₆H₅ | Me | Me |
| 54 | CN | C₆H₅ | H | H |
| 55 | H | | H | H |
| 56 | H | (S)- | H | H |
| 57 | H | | H | H |
| 58 | H | | H | H |
| 59 | OMe | (S)- | H | H |
| 60 | CHF—CH₃ | (S)- | H | H |
| 61 | CH₂F | (S)- | Me | Me |
| 62 | OH | (±)-2-thienyl | H | H |
| 63 | H | (R)-C₆H₅ | H | H |
| 64 | CH₃ | (S)-C₆H₅ | H | H |
| 65 | H | p-fluoro-C₆H₄ | H | H |
| 66 | H | 4-chloro-C₆H₄ | H | H |
| 67 | OH | (S)-4-chloro-C₆H₄ | H | H |
| 68 | H | 4-methyl-C₆H₄ | H | H |
| 69 | H | (S)-4-CH₃—C₆H₄ | H | H |
| 70 | OH | cyclohexyl | H | H |
| 71 | H | 3-benzo[b]thiophene | Me | Me |
| 72 | H | 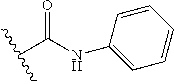 [SMILES: *C(=O)Nc1ccccc1] | H | H |

### Example 73. Preparation of a Gamma Amino Acid Version. (E73)

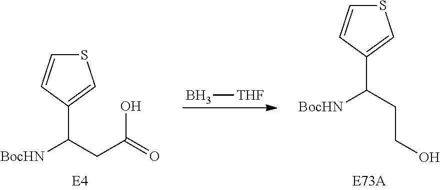

To (±)-2-(tert-butoxycarbonylamino)-2-(thiophen-3-yl)propanoic acid (E4) in THF at 0° C. was added BH₃-THF dropwise. The solution was allowed to warm to room temperature and stirred for an additional 2 hours. The solution was cooled to 0° C., quenched with AcOH (10%)/MeOH, and evaporated. Column chromatography (SiO₂, EtOAc) gave pure tert-butyl 3-hydroxy-1-(thiophen-3-yl)propylcarbamate (E73A).

### Preparation of 3-(tert-butoxycarbonylamino)-3-(thiophen-3-yl)propyl 4-methylbenzenesulfonate (E73B)

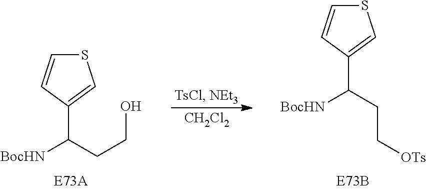

To tert-butyl 2-hydroxy-1-(thiophen-3-yl)ethylcarbamate (E73A) in CH₂Cl₂ was added NEt₃, DMAP, and TsCl. The solution was stirred at room temperature for 3 hours and then poured into NH₄Cl (sat) and extracted with EtOAc, dried (Na₂SO₄), filtered, and evaporated. Column chromatography (SiO₂, 30% EtOAc/Hexanes) gave pure 3-(tert-butoxycarbonylamino)-3-(thiophen-3-yl) propyl 4-methylbenzenesulfonate (E73B).

### Preparation of tert-butyl 3-cyano-1-(thiophen-3-yl)propylcarbamate (E73C)

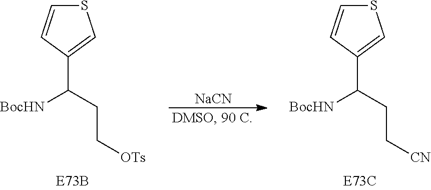 [SMILES: Cc1ccc(S(=O)(=O)OCCC(NC(=O)OC(C)(C)C)c2ccsc2)cc1 ; "NaCN" ; "DMSO, 90 C." ; CC(C)(C)OC(=O)NC(CCC#N)c1ccsc1]

To 3-(tert-butoxycarbonylamino)-3-(thiophen-3-yl)propyl 4-methyl benzenesulfonate (E73B) in DMSO was added NaCN, and the solution was heated to 90° C. for 2 hours. The reaction was cooled, poured into NaCl₍ₛₐₜ₎, and extracted with EtOAc. The organics were dried (Na₂SO₄), filtered, and evaporated. Column chromatography (SiO₂, 25% EtOAc/Hexanes) gave pure tert-butyl 2-cyano-1-(thiophen-3-yl)ethylcarbamate (E73C).

### Preparation of 3-(tert-butyoxcarbonylamino)-3-(thiophen-3-yl)propanoic acid (E73D)

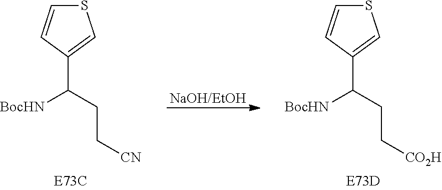 [SMILES: CC(C)(C)OC(=O)NC(CCC#N)c1ccsc1 ; "NaOH/EtOH" ; CC(C)(C)OC(=O)NC(CCC(=O)O)c1ccsc1]

To tert-butyl 3-cyano-1-(thiophen-3-yl)propylcarbamate (E3) in EtOH was added NaOH (2M) and the solution was heated to 90° C. for 4 hours. The reaction was cooled, acidified with HCl, and extracted with EtOAc. The organics were dried (Na₂SO₄) and evaporated to give pure 4-((tert-butoxycarbonyl)amino)-4-(thiophen-3-yl)butanoic acid (E73D).

### Preparation of tert-butyl 4-(benzoisothiazol-6-ylamino)-4-oxo-1-(thiophen-3-yl)butylcarbamate (E73E)

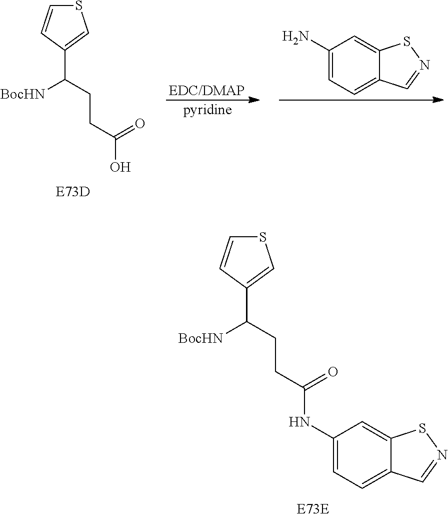 [SMILES: CC(C)(C)OC(=O)NC(CCC(=O)O)c1ccsc1 ; "pyridine" ; "EDC/DMAP" ; Nc1ccc2cnsc2c1 ; CC(C)(C)OC(=O)NC(CCC(=O)Nc1ccc2cnsc2c1)c1ccsc1]

To 4-((tert-butoxycarbonyl)amino)-4-(thiophen-3-yl) butanoic acid (E73D) in pyridine was added EDC, DMAP and 6-aminobenzoisothiazole and the solution was stirred for 10 hours at room temperature. The mixture was poured into NaHCO₃ (sat) and extracted with EtOAc, dried (Na₂SO₄), filtered and evaporated. Column chromatography (SiO₂, 5% MeOH/CH₂Cl₂) gave pure tert-butyl 4-(benzoisothiazol-6-ylamino)-4-oxo-1-(thiophen-3-yl)butylcarbamate (E73E).

### Preparation of 4-amino-N-(benzoisothiazol-6-yl)-4-(thiophen-3-yl)butanamide dihydrochloride (E73)

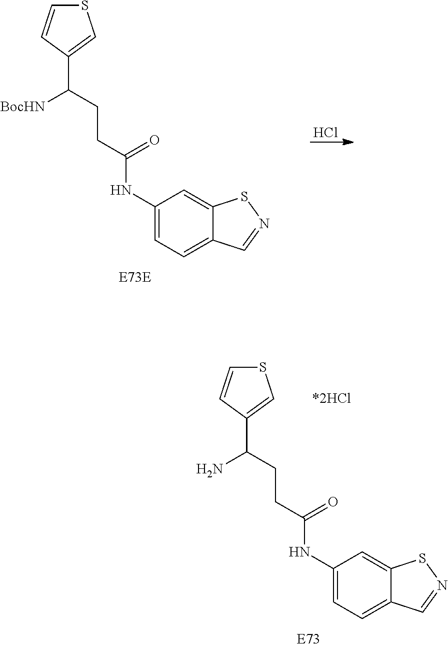 [SMILES: CC(C)(C)OC(=O)NC(CCC(=O)Nc1ccc2cnsc2c1)c1ccsc1 ; Cl ; NC(CCC(=O)Nc1ccc2cnsc2c1)c1ccsc1]

To tert-butyl 4-(benzoisothiazol-6-ylamino)-4-oxo-1-(thiophen-3-yl)butylcarbamate (E73E) in CH₂Cl₂ was added HCl (4N in dioxane), and the solution was stirred for 8 hours. The solvents were evaporated to give 4-amino-N-(benzoisothiazol-6-yl)-4-(thiophen-3-yl)butanamide dihydrochloride (E73).

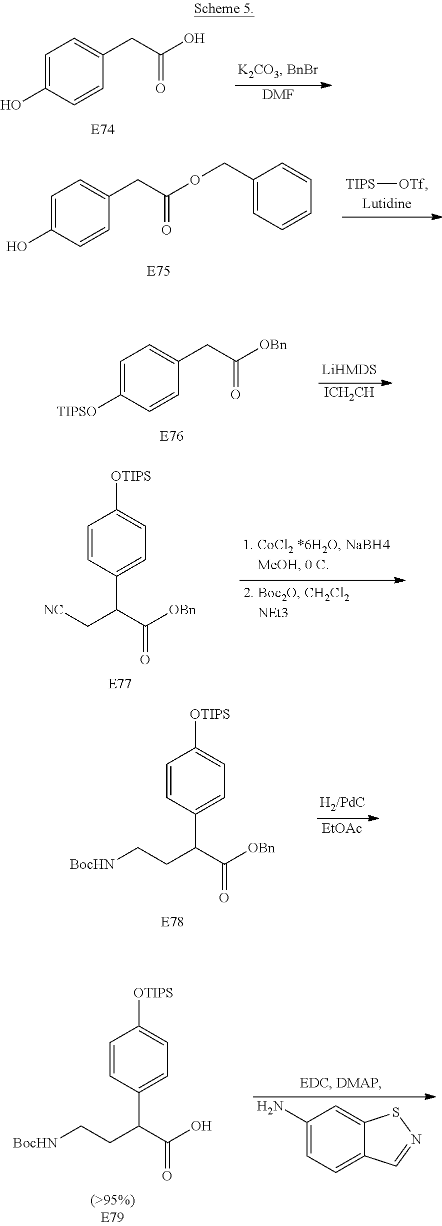

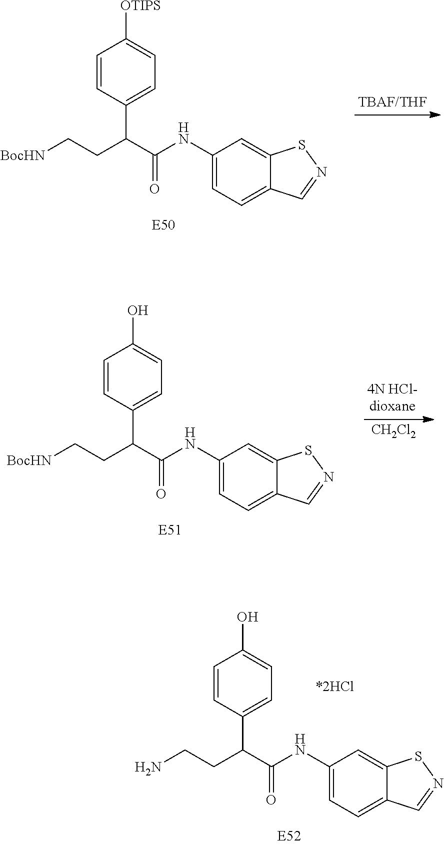

### Preparation of benzyl 2-(4-hydroxyphenyl)acetate (E75)

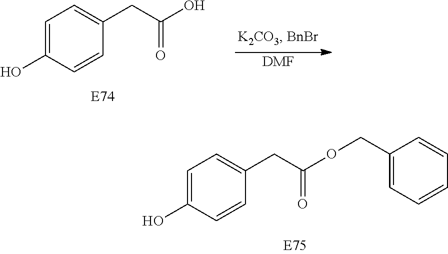

To 2-(4-hydroxyphenyl)acetic acid in DMF cooled to 0° C. was added K₂CO₃ and the solution was stirred for 30 minutes. Then, benzyl bromide was added and the solution stirred at 0° C. and was allowed to slowly warm to 15-20° C. After all the ice was melted the solution was poured into NH₄Cl₍ₛₐₜ₎ and extracted with EtOAc. The combined organics were dried (Na₂SO₄), filtered and evaporated. Column chromatography (SiO₂, 0-35% EtOAc/Hex) gave pure benzyl 2-(4-hydroxyphenyl)acetate (E75).

### Preparation of benzyl 2-(4-(triisopropylsiloxy)phenyl)acetate (E76)

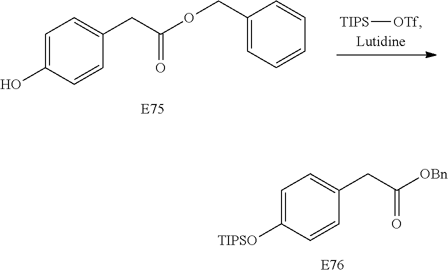

To benzyl 2-(4-hydroxyphenyl)acetate (E75) in CH₂Cl₂ at 0° C. was added 2,6-lutidine and TIPS-OTf and the solution stirred for 2.5 hours at 0° C. The mixture was poured into NH₄Cl (sat) and extracted with CH₂C₂. The combined organics were dried (Na₂SO₄), filtered and evaporated. Column chromatography (SiO₂, 0-15% EtOAc/Hex) gave pure benzyl 2-(4-(triisopropylsilyloxy) phenyl)acetate (E76).

### Preparation of benzyl 3-cyano-2-(triisopropylsilyloxy) phenyl) propanoate (E77)

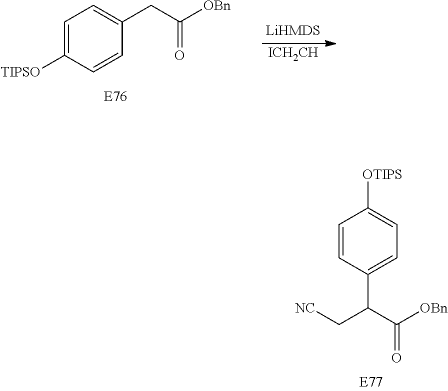

To a solution of LiHMDS in THF at −78° C. was added a solution of benzyl 2-(4-(triisopropylsiloxy)phenyl)acetate (E76) in THF also cooled to approximately −78° C. and this mixture was allowed to stir at −78° C. for 30 minutes. Iodoacetonitrile was then added and the mixture was warmed to 0° C. and stirred for 2 hours. The mixture was poured into NH₄Cl₍ₛₐₜ₎ and extracted with EtOAc. The combined organics were dried (Na₂SO₄), filtered and evaporated. Column chromatography (SiO₂, 0-25% EtOAc/Hex) gave pure benzyl 3-cyano-2-(triisopropylsilyloxy)phenyl)propanoate (E77).

### Preparation of benzyl 4-(tertbutoxycarbonylamino)-2-(4-(triisopropylsilyloxy)phenyl)butanoate (E78)

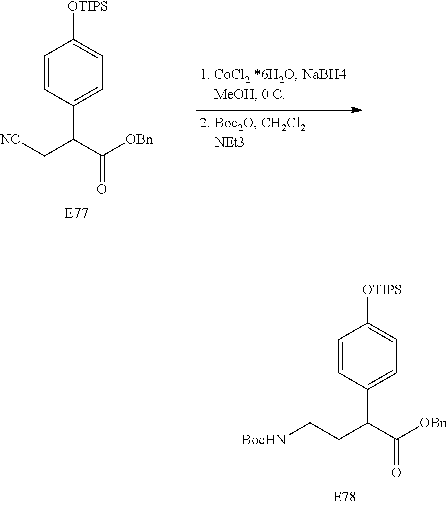

To a solution of benzyl 3-cyano-2-(triisopropylsilyloxy) phenyl)propanoate (E77) in MeOH cooled to 0° C. was added CoCl₂.6H₂O and NaBH₄ and the solution was allowed to stir for 20 minutes. Then, HCl (1.25 N in MeOH) was added and the solution stirred an additional 20 minutes at 0° C. The solvents were evaporated and the mixture was taken up in CH₂Cl₂ and cooled to 0° C. Boc₂O and NEt₃ were added and the solution stirred at 0° C. for 1.5 hours. The mixture was poured into NH₄Cl₍ₛₐₜ₎ and extracted with CH₂C₂. The combined organics were dried (Na₂SO₄), filtered and evaporated. Column chromatography (SiO₂, 10-20% EtOAc/hexanes) gave pure benzyl 4-(tert-butoxycarbonylamino)-2-(4-(triisopropylsilyloxy)phenyl)butanoate (E78).

### Preparation of 4-(tert-butoxycarbonylamino)-2-(4-(triisopropylsilyloxy))butanoic acid (E79)

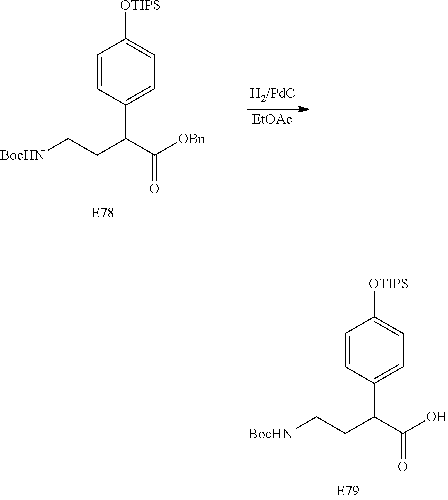

To benzyl 4-(tert-butoxycarbonylamino)-2-(4-(triisopropylsilyloxy)phenyl)butanoate (E78) in EtOAc was added Pd/C (10%) and the solution was kept under a H₂ atmosphere for 2 hours. The mixture was filtered over Celite and the solvent was evaporated to give 4-(tert-butoxycarbonylamino)-2-(4-(triisopropylsilyloxy)phenyl)butanoic acid (E79).

### Preparation of tert-butyl (4-(benzoisothiazol-6-ylamino)-4-oxo-3-(4-((triisopropylsilyl)oxy)phenyl)butyl)carbamate (E80)

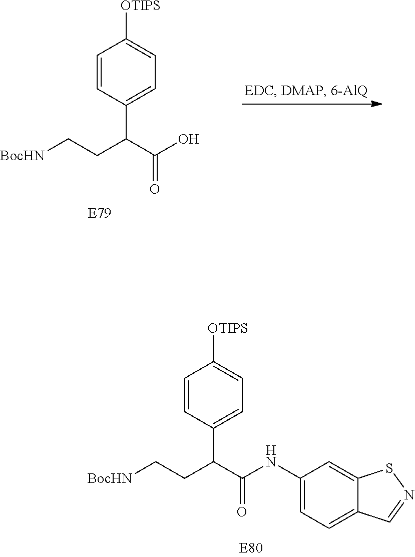

To 4-(tert-butoxycarbonylamino)-2-(4-(triisopropylsilyloxy) phenyl) butanoic acid (E79) in pyridine was added EDC, DMAP and benzoisothiazol-6-amine and the solution was stirred at room temperature overnight. The mixture was poured into NaHCO₃₍ₛₐₜ₎ and extracted with EtOAc. The combined organics were dried (Na₂SO₄), filtered and evaporated. Column chromatography (SiO₂, 4% MeOH/CH₂Cl₂) gave pure tert-butyl (4-(benzoisothiazol-6-ylamino)-4-oxo-3-(4-((triisopropylsilyl)oxy)phenyl)butyl)carbamate (E80).

### Preparation of tert-butyl (4-(benzo[d]isothiazol-6-ylamino)-3-(4-hydroxyphenyl)-4-oxobutyl)carbamate (E81)

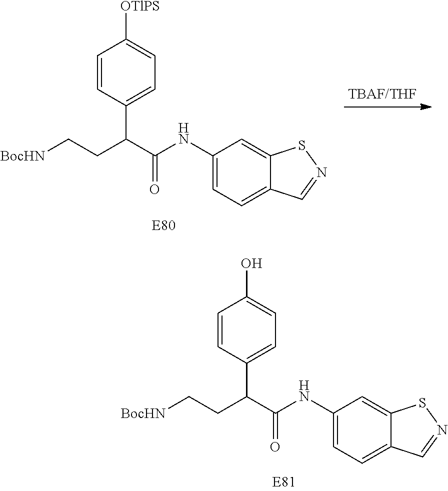

To tert-butyl (4-(benzoisothiazol-6-ylamino)-4-oxo-3-(4-((triisopropyl silyl)oxy)phenyl)butyl) carbamate (E80). in THF at 0° C. was added TBAF and the solution was stirred at 0° C. for 30 minutes. The solution was poured into NH₄C (sat) and extracted with EtOAc. The combined organics were dried (Na₂SO₄), filtered and evaporated. Column chromatography (SiO₂, 5-8% MeOH/CH₂Cl₂) gave pure tert-butyl(4-(benzoisothiazol-6-ylamino)-3-(4-hydroxyphenyl)-4-oxobutyl) carbamate (E81).

### Preparation of 4-amino-N-(benzoisothiazol-6-yl)-2-(4-hydroxyphenyl)butanamide dihydrochloride (E82)

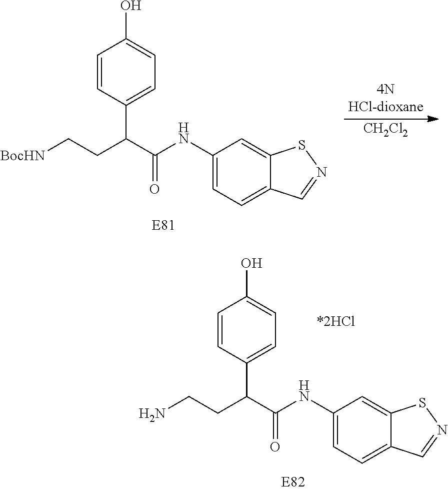

To tert-butyl (4-(benzoisothiazol-6-ylamino)-3-(4-hydroxyphenyl)-4-oxobutyl)carbamate (E81) in CH₂Cl₂ was added HCl (4N in dioxane) and 2 drops of H₂O and the solution was stirred overnight at room temperature. The solvents were evaporated to give 4-amino-N-(benzoisothiazol-6-yl)-2-(4-hydroxyphenyl) butanamide dihydrochloride (E82).

### Examples 83-110

Using the general procedures shown for Examples 73-82, the following compounds of Table 3 can be synthesized from the corresponding 6-aminobenzoisothiazole.

Thus, in some embodiments, provided herein are compounds of the following Formula:

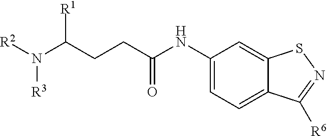

or a pharmaceutically acceptable salt thereof

wherein

R¹ is C₆H₅, (S)—C₆H₅, (R)—C₆H5, 4-F—C₆H₄, 4-C₁-C₆H₄, cyclopropyl, 3-thienyl, (S)-3-thienyl, or cyclohexyl;

R² is H or CH₃;

R³ is H, CH₃, or CH₂C₆H₅; and

R⁶ is H, OH, OMe, OEt, CN, or CH₃.

In some embodiments, provided herein are compounds of the following Formula:

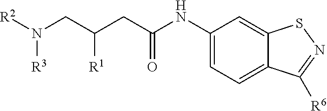

or a pharmaceutically acceptable salt thereof

wherein

R¹ is C₆H₅, 4-C₁-C₆H₄, CH₂C₆H5, 3-thienyl, 2-thienyl, or p-chloro benzyl;

R² is H or CH₃;

R³ is H or CH₃; and

R⁶ is H, CH₂F, CHFCH₃, or OH.

In some embodiments, provided herein are compounds of the following Formula:

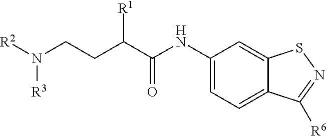

or a pharmaceutically acceptable salt thereof

wherein

R¹ is C₆H₅, 4-C₁-C₆H₄, 4-CH₃—C₆H₄, 4-CF₃—C₆H₄,

R² is H;

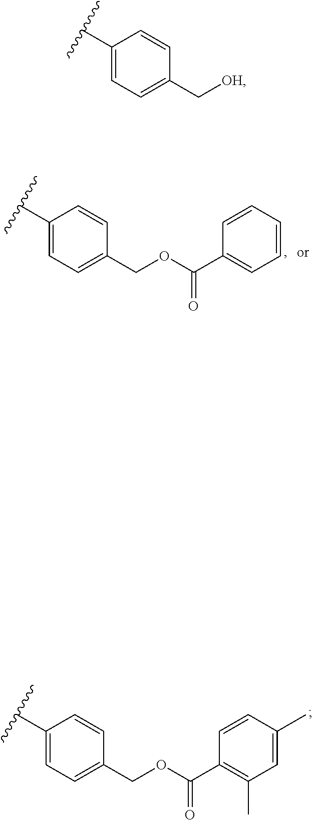 [SMILES: *c1ccc(CO)cc1 ; *c1ccc(COC(=O)c2ccccc2)cc1 ; *c1ccc(COC(=O)c2ccc(C)cc2C)cc1]

R³ is H or CH₃; and

R⁶ is H.

TABLE 3

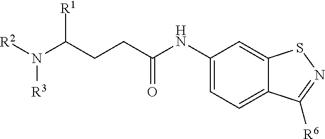

| Example | R⁶ | R¹ | R³ | R² |
|---|---|---|---|---|
| 83 | H | C₆H₅ | H | H |
| 84 | OH | C₆H₅ | H | H |
| 85 | H | (S)-C₆H₅ | H | H |
| 86 | OMe | (R)-C₆H₅ | H | H |
| 87 | OEt | (S)-C₆H₅ | H | H |
| 88 | H | (S)-C₆H₅ | CH₃ | CH₃ |
| 89 | H | 4-F—C₆H₄ | H | H |
| 90 | H | 4-Cl—C₆H₄ | CH₃ | H |
| 91 | CN | cyclopropyl | CH₃ | H |
| 92 | CH₃ | 3-thienyl | H | H |
| 93 | H | (S)-3-thienyl | H | H |
| 94 | OH | cyclohexyl | CH₃ | CH₃ |
| 97 | H | C₆H₅ | CH₂C₆H₅ | H |

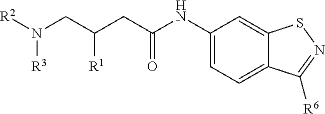

| Example | R⁶ | R¹ | R³ | R² |
|---|---|---|---|---|
| 95 | H | C₆H₅ | H | H |
| 96 | CH₂F | C₆H₅ | CH₃ | H |
| 98 | CHFCH₃ | 4-chloro-C₆H₄ | H | H |
| 99 | OH | CH₂C₆H₅ | CH₃ | H |
| 100 | H | 3-thienyl | CH₃ | H |
| 101 | OH | 2-thienyl | CH₃ | CH₃ |
| 102 | H | p-chloro-benzyl | H | H |

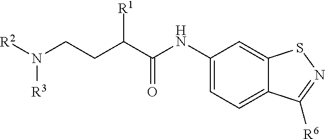

| Example | R⁶ | R¹ | R³ | R² |
|---|---|---|---|---|
| 103 | H | C₆H₅ | H | H |
| 104 | H | 4-Cl—C₆H₄ | H | H |
| 105 | H | 4-CH₃—C₆H₄ | H | H |
| 106 | H | 4-CH₃—C₆H₄ | CH₃ | H |
| 107 | H | 4-CF₃—C₆H₄ | H | H |
| 108 | H | 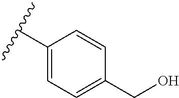 [SMILES: *c1ccc(CO)cc1] | H | H |
| 109 | H | 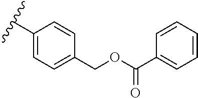 [SMILES: *c1ccc(COC(=O)c2ccccc2)cc1] | H | H |
| 110 | H | 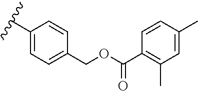 [SMILES: *c1ccc(COC(=O)c2ccc(C)cc2C)cc1] | H | H |

Using the general procedure shown for Example 73 and Examples 74-82, the following the compounds 111-138 of Table 4 can be made.

TABLE 4

| | |
|---|---|
| 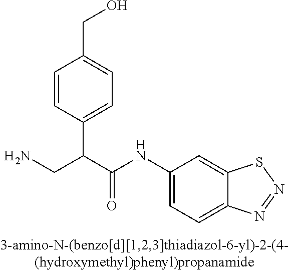 [SMILES: NCC(C(=O)Nc1ccc2nnsc2c1)c1ccc(CO)cc1] | 111 |
| 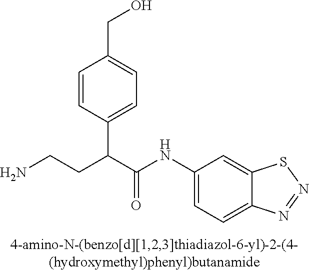 [SMILES: NCCC(C(=O)Nc1ccc2nnsc2c1)c1ccc(CO)cc1] | 112 |
| 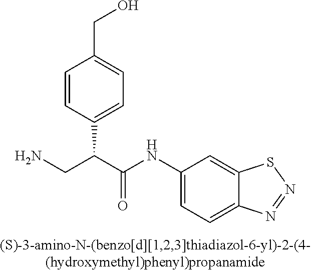 [SMILES: NC[C@@H](C(=O)Nc1ccc2nnsc2c1)c1ccc(CO)cc1] | 113 |
| 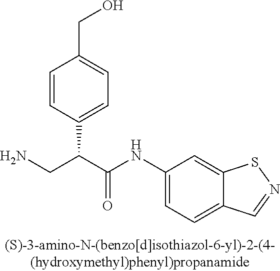 [SMILES: NC[C@@H](C(=O)Nc1ccc2cnsc2c1)c1ccc(CO)cc1] | 114 |
| 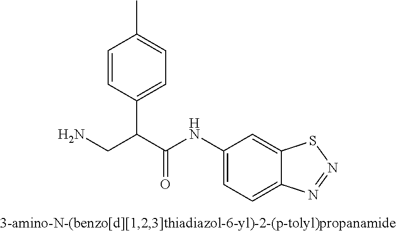 [SMILES: Cc1ccc(C(CN)C(=O)Nc2ccc3nnsc3c2)cc1] | 115 |
| 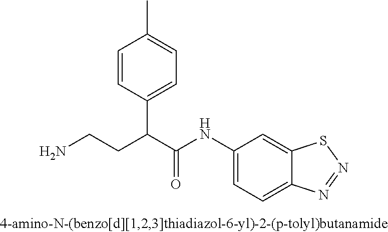 [SMILES: Cc1ccc(C(CCN)C(=O)Nc2ccc3nnsc3c2)cc1] | 116 |
| 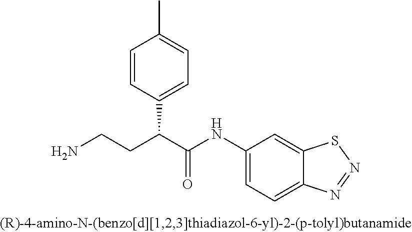 [SMILES: Cc1ccc([C@@H](CCN)C(=O)Nc2ccc3nnsc3c2)cc1] | 117 |
| 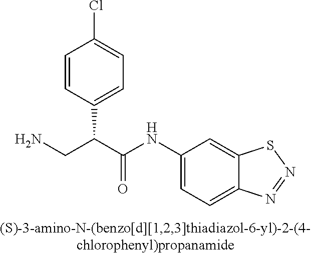 [SMILES: NC[C@@H](C(=O)Nc1ccc2nnsc2c1)c1ccc(Cl)cc1] | 118 |
| 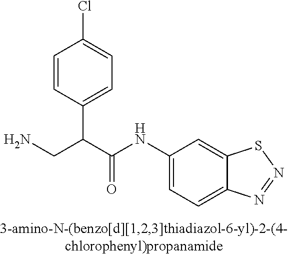 [SMILES: NCC(C(=O)Nc1ccc2nnsc2c1)c1ccc(Cl)cc1] | 119 |
| 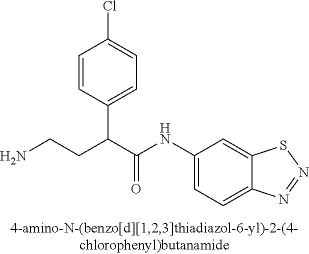 [SMILES: NCCC(C(=O)Nc1ccc2nnsc2c1)c1ccc(Cl)cc1] | 120 |
| 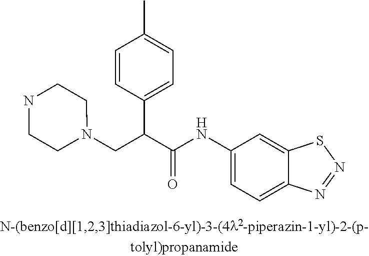 [SMILES: Cc1ccc(C(CN2CCNCC2)C(=O)Nc2ccc3nnsc3c2)cc1] | 121 |
| 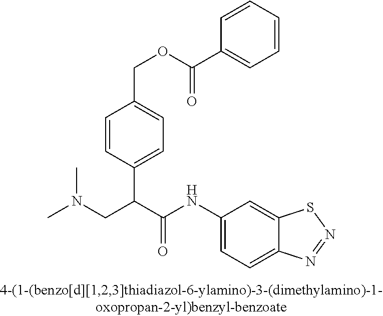 [SMILES: CN(C)CC(C(=O)Nc1ccc2nnsc2c1)c1ccc(COC(=O)c2ccccc2)cc1] | 122 |
| 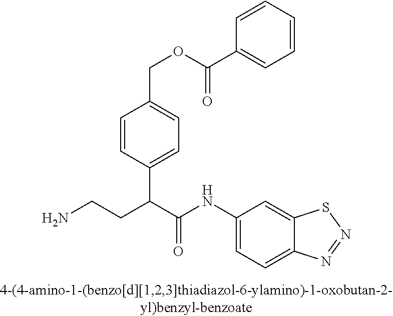 [SMILES: NCCC(C(=O)Nc1ccc2nnsc2c1)c1ccc(COC(=O)c2ccccc2)cc1] | 123 |
| 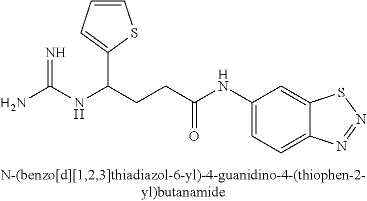 [SMILES: N=C(N)NC(CCC(=O)Nc1ccc2nnsc2c1)c1cccs1] | 124 |
| 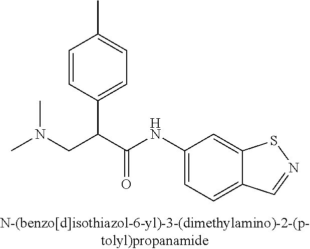 [SMILES: Cc1ccc(C(CN(C)C)C(=O)Nc2ccc3cnsc3c2)cc1] | 125 |
| 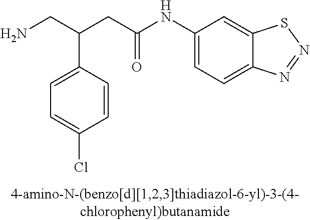 [SMILES: NCC(CC(=O)Nc1ccc2nnsc2c1)c1ccc(Cl)cc1] | 126 |
| 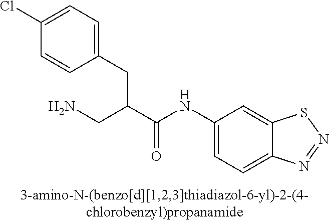 [SMILES: NCC(Cc1ccc(Cl)cc1)C(=O)Nc1ccc2nnsc2c1] | 127 |
| 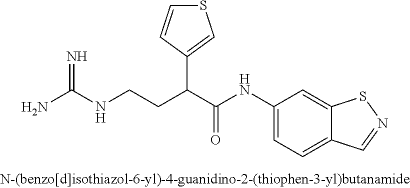 [SMILES: N=C(N)NCCC(C(=O)Nc1ccc2cnsc2c1)c1ccsc1] | 128 |
| 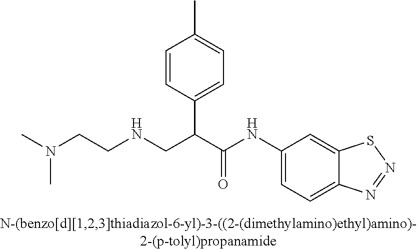 [SMILES: Cc1ccc(C(CNCCN(C)C)C(=O)Nc2ccc3nnsc3c2)cc1] | 129 |
| 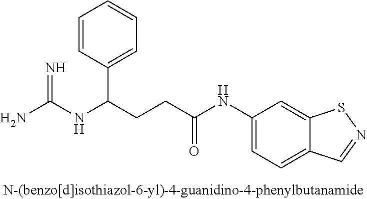 [SMILES: N=C(N)NC(CCC(=O)Nc1ccc2cnsc2c1)c1ccccc1] | 130 |
| 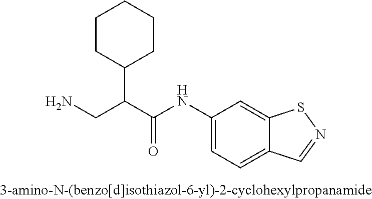 [SMILES: NCC(C(=O)Nc1ccc2cnsc2c1)C1CCCCC1] | 131 |
| 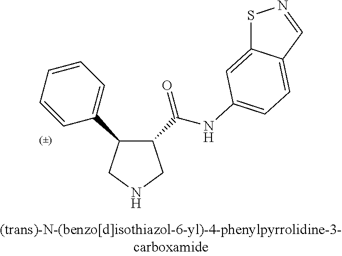 | 132 |
| 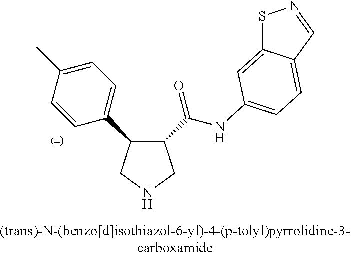 | 133 |
| 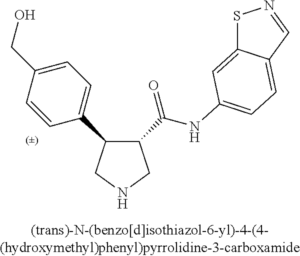 | 134 |
| 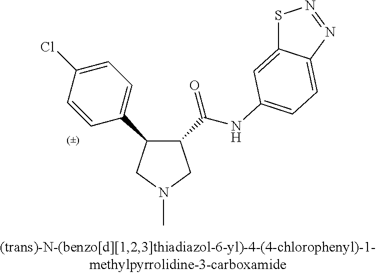 | 135 |

The following preparations illustrate procedures for the preparation of intermediates and methods for the preparation of cyclopropyl benzoisothiazole amides.

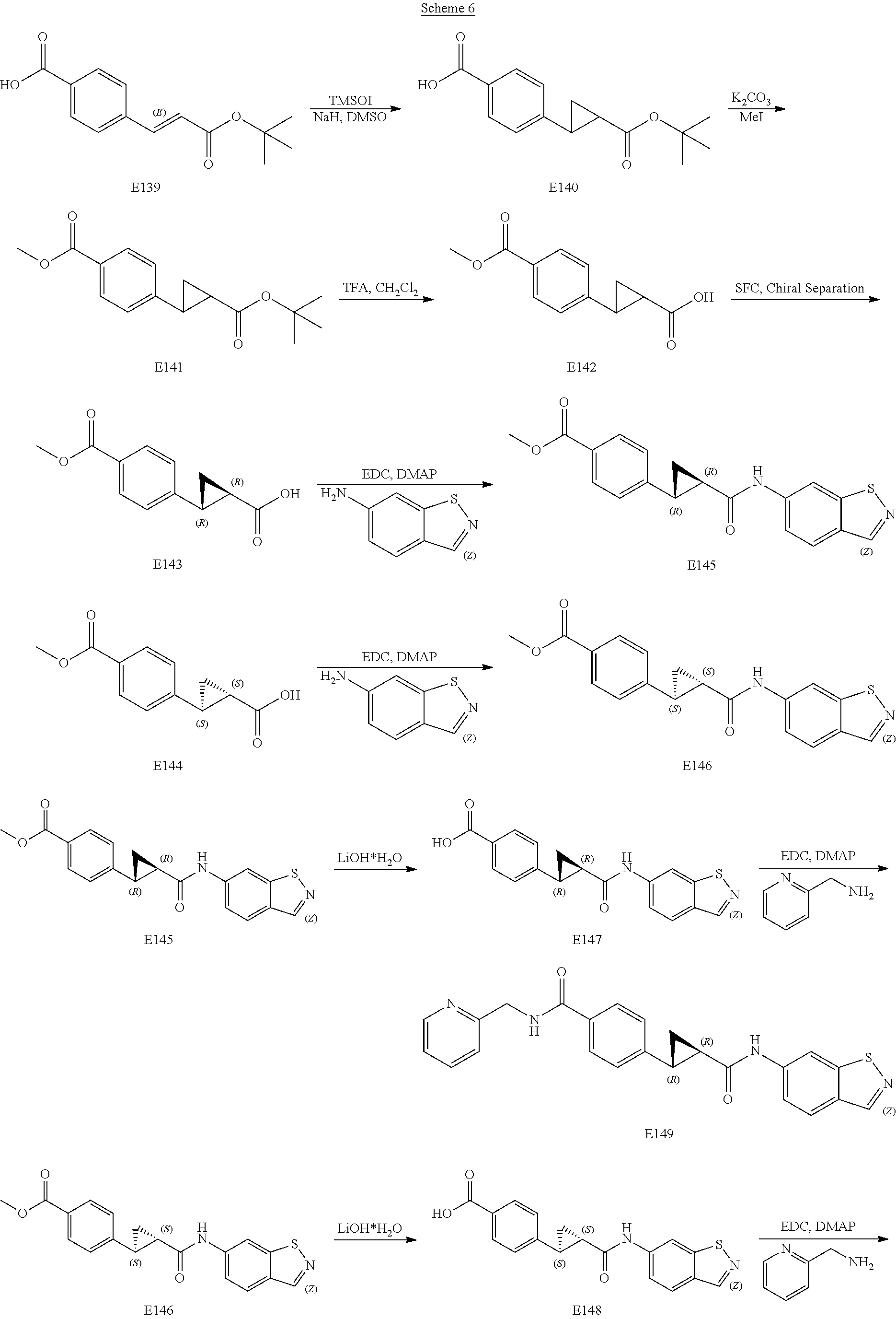

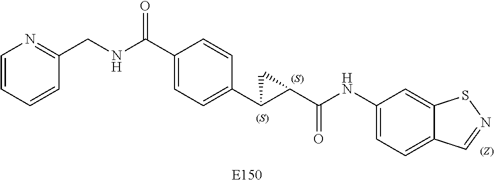

### Preparation of 4-(2-(tert-butoxycarbonyl)cyclopropyl)benzoic acid (E140)

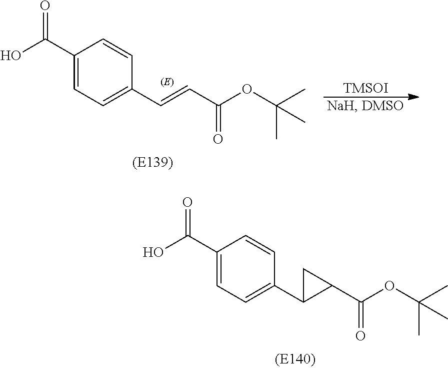

To TMSOI in DMSO was added NaH and solution was stirred for one hour under N₂. (E)-4-(3-(tert-butoxy)-3-oxoprop-1-en-1-yl)benzoic acid (E139) dissolved in DMSO was added and the a solution was stirred for 3 hours at room temperature. The mixture was poured into cold EtOAc and HCl (1N) and extracted with EtOAc. The organics were dried (Na₂SO₄), filtered and evaporated. Column chromatography 500 MeOH— CH₂Cl₂ gave pure 4-(2-(tert-butoxycarbonyl)cyclopropyl)benzoic acid (E140).

### Preparation of methyl 4-(2-(tert-butoxycarbonyl)cyclopropyl)benzoate (E141)

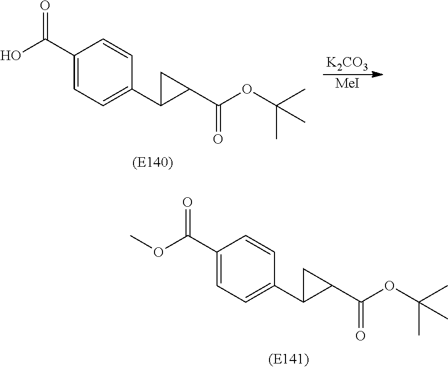

To 4-(2-(tert-butoxycarbonyl)cyclopropyl)benzoic acid (E140), in DMF cooled to 0° C. was added K₂CO₃ and solution was stirred for 30 min at 0° C. under N₂. Then, methyl iodide was added and the solution was warmed and stirred at room temperature for 2-3 hours. The reaction was poured into EtOAc/HCl (1N) and extracted with EtOAc, dried (Na₂SO₄), filtered and evaporated. Silica gel column chromatography using 0-5% EtOAc in Hexanes provided pure methyl 4-(2-(tert-butoxycarbonyl)cyclopropyl)benzoate (E141).

### Preparation of 2-(4-(methoxycarbonyl)phenyl)cyclopropane-1-carboxylic acid (E142)

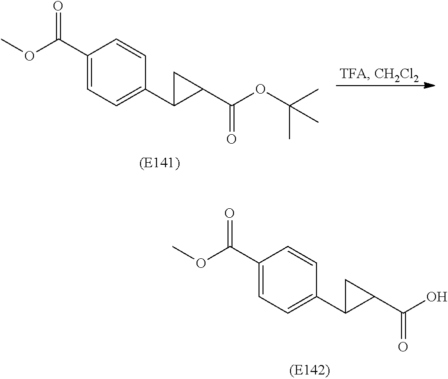

To methyl 4-(2-(tert-butoxycarbonyl)cyclopropyl)benzoate (E141) in CH₂Cl₂ was added TFA and solution was 3-6 hours at room temperature. The solvents were evaporated and column chromatography 0-5% MeOH—CH₂Cl₂ gave pure 2-(4-(methoxycarbonyl)phenyl)cyclopropane-1-carboxylic acid (E142).

### Preparation of methyl 4-((1R,2R)-2-(benzoisothiazol-6-ylcarbamoyl) cyclopropyl)benzoate (E145)

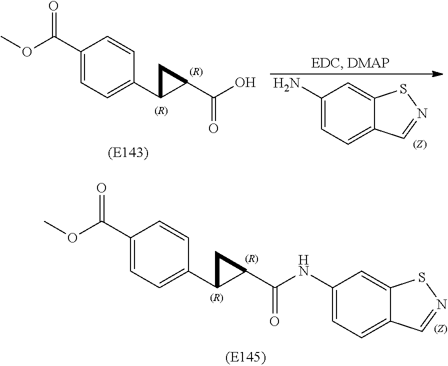

To (1R,2R)-2-(4-(methoxycarbonyl)phenyl)cyclopropane-1-carboxylic acid (E143) in pyridine was added EDC, DMAP and benzo[d]isothiazol-6-amine and the solution was stirred under N₂ overnight. The mixture was poured into NaHCO₃ and extracted with EtOAc, dried (Na₂SO₄) filtered and evaporated. Column chromatography 5-8% MeOH—CH₂Cl₂ gave 4-((1R,2R)-2-(benzoisothiazol-6-ylcarbamoyl)cyclopropyl) benzoate (E145).

### Preparation of methyl 4-((1S,2S)-2-(benzoisothiazol-6-ylcarbamoyl)cyclopropyl) benzoate (E145)

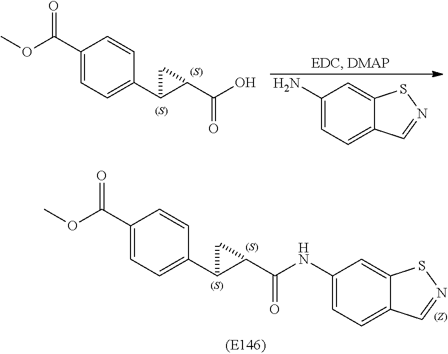

To (1S,2S)-2-(4-(methoxycarbonyl)phenyl)cyclopropane-1-carboxylic acid (E144) in pyridine was added EDC, DMAP and benzoisothiazole-6-amine and the solution was stirred under N₂ overnight. The mixture was poured into NaHCO₃ and extracted with EtOAc, dried (Na₂SO₄) filtered and evaporated. Column chromatography 5-8% MeOH—CH₂Cl₂ gave 4-((1S,2S)-2-(benzoisothiazol-6-ylcarbamoyl)cyclopropyl) benzoate (E146).

### Preparation of 4-((1R,2R)-2-(benzoisothiazol-6-ylcarbamoyl)cyclopropyl)benzoic acid (E147)

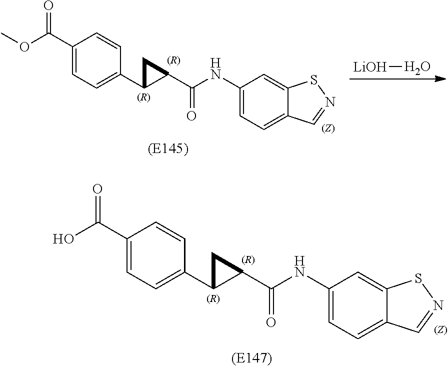

To 4-((1R,2R)-2-(benzo[d]isothiazol-6-ylcarbamoyl)cyclopropyl)benzoate (E145) in THF-H₂O at 0° C. was added LiOH*H₂O and the solution was stirred at room temperature for 24 hours. The reaction was acidified to pH 5 with HCl (1N) and the solids were filtered to give 4-((1R,2R)-2-(benzoisothiazol-6-ylcarbamoyl) cyclopropyl)benzoic acid (E147).

### Preparation of 4-((1S,2S)-2-(benzoisothiazol-6-ylcarbamoyl)clopropyl)benzoic acid (E148)

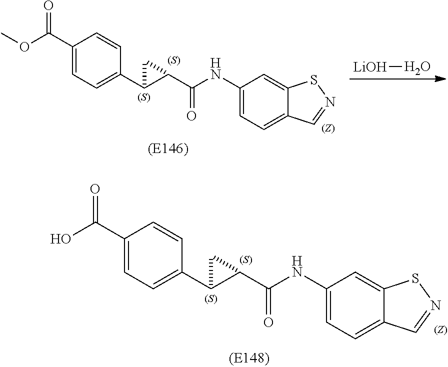

To 4-((1S,2S)-2-(benzoisothiazol-6-ylcarbamoyl)cyclopropyl)benzoate (E145) in THF-H₂O at 0° C. was added LiOH.H₂O and the solution was stirred at room temperature for 24 hours. The reaction was acidified to pH 5 with HCl (1N) and the solids were filtered to give 4-((1S,2S)-2-(benzoisothiazol-6-ylcarbamoyl) cyclopropyl)benzoic acid (E148).

### Preparation of 4-((R,2R)-2-(benzoisothiazol-6-ylcarbamoyl)cyclopropyl)-N-(pyridin-2-ylmethyl)benzamide (E149)

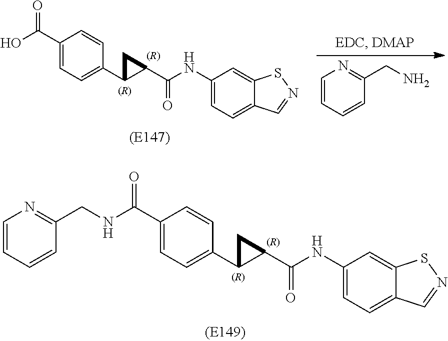 [0435] a. To 4-((1R,2R)-2-(benzoisothiazol-6-ylcarbamoyl)cyclopropyl)benzoic acid (E147), in pyridine was added EDC, DMAP and 2-picolylamine and the solution was stirred under N₂ at room temperature overnight. The reaction was poured into EtOAc/NaHCO₃ (sat) and extracted with EtOAc, dried (Na₂SO₄), filtered and evaporated. Column chromatography 5-8% MeOH—CH₂Cl₂ gave pure 4-((1R,2R)-2-(benzoisothiazole-6-ylcarbamoyl)cyclopropyl)-N-(pyridin-2-ylmethyl)benzamide (E149).

### Preparation of 4-((S,2S)-2-(benzoisothiazol-6-ylcarbamoyl)cyclopropyl)-N-(pyridin-2-ylmethyl)benzamide (E150)

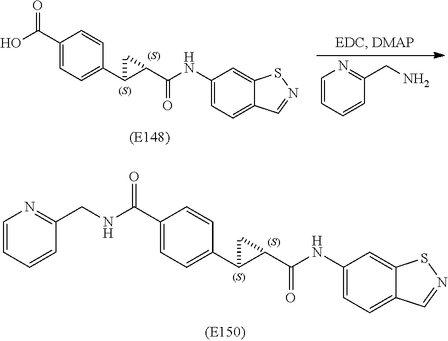

To 4-((1S,2S)-2-(benzoisothiazol-6-ylcarbamoyl)cyclopropyl)benzoic acid (E148), in pyridine was added EDC, DMAP and 2-picolylamine and the solution was stirred under N₂ at room temperature overnight. The reaction was poured into EtOAc/NaHCO₃ (sat) and extracted with EtOAc, dried (Na₂SO₄), filtered and evaporated. Column chromatography 5-8% MeOH—CH₂Cl₂ gave pure 4-((1S,2S)-2-(benzoisothiazol-6-ylcarbamoyl)cyclopropyl)-N-(pyridin-2-ylmethyl)benzamide (E150).

Using commercially available compounds and largely the procedures set forth in Examples 140-150 and substituting the appropriate starting materials, the compounds 151-195 of Table 5 can be made.

Thus, in some embodiments, provided herein are compounds of the following Formula:

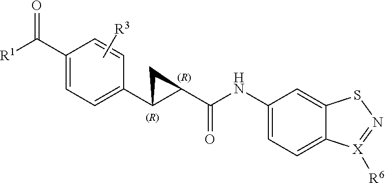

or a pharmaceutically acceptable salt thereof

wherein

R¹ is OH, NH₂, OMe,

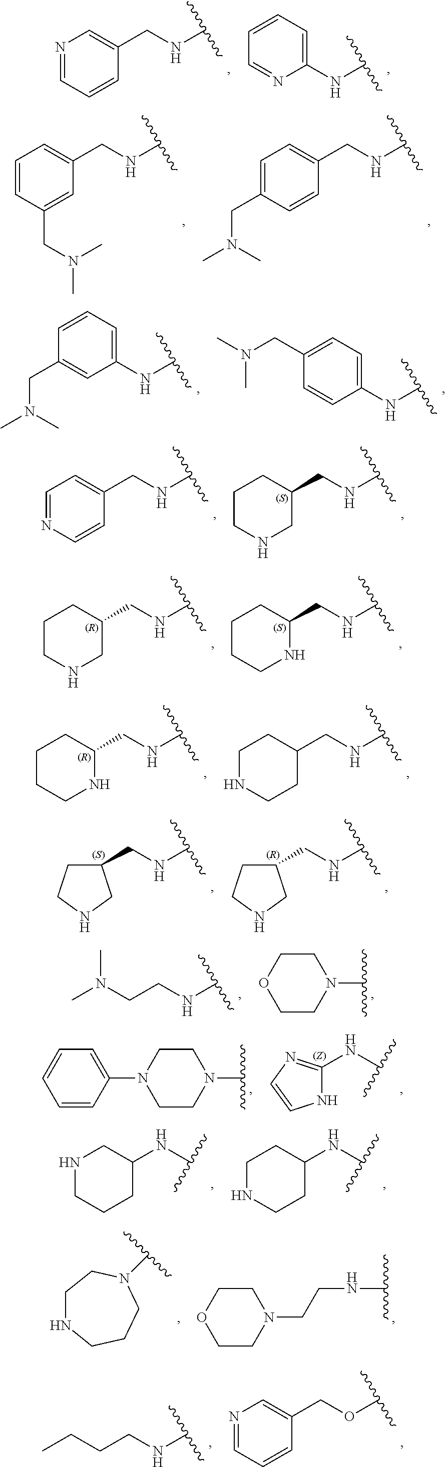

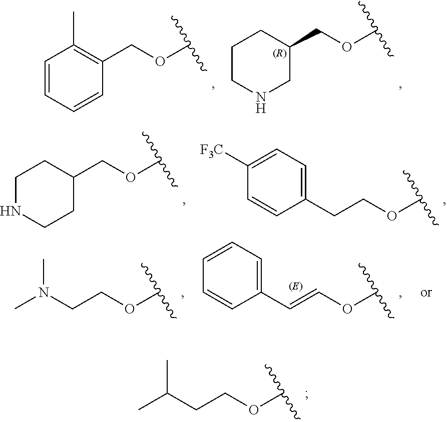

R³ is H, 3-F, 2-F, or 2-Cl; and

X is N and R⁶ is null, or X is C and R⁶ is H or OH.

TABLE 5

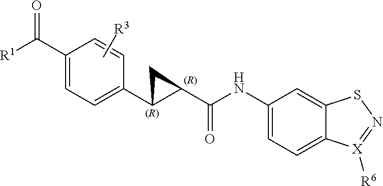

| Example | R¹ | R⁶ | R³ | X |
|---|---|---|---|---|
| 151 | OH | H | H | C |
| 152 | OH | — | 3-F | N |
| 153 | NH₂ | OH | H | C |
| 154 | NH₂ | H | H | C |
| 155 | OMe | — | 2-F | N |
| 156 | OMe | H | H | C |
| 157 | 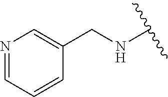 [SMILES: *NCc1cccnc1] | OH | H | C |
| 158 | 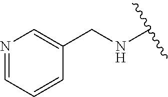 [SMILES: *NCc1cccnc1] | — | H | N |
| 159 | 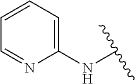 [SMILES: *Nc1ccccn1] | OH | 2-Cl | C |
| 160 | 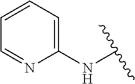 [SMILES: *Nc1ccccn1] | — | H | N |
| 161 | 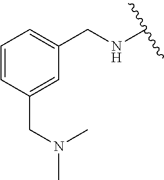 [SMILES: *NCc1cccc(CN(C)C)c1] | H | H | C |
| 162 | 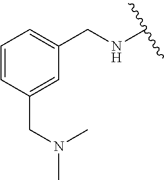 [SMILES: *NCc1cccc(CN(C)C)c1] | — | 3-F | N |
| 163 | 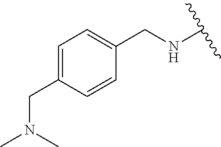 [SMILES: *NCc1ccc(CN(C)C)cc1] | H | H | C |
| 164 | 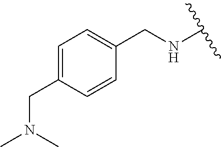 [SMILES: *NCc1ccc(CN(C)C)cc1] | H | H | C |
| 165 | 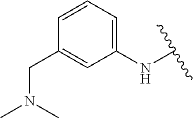 [SMILES: *Nc1cccc(CN(C)C)c1] | — | H | N |
| 166 | 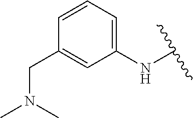 [SMILES: *Nc1cccc(CN(C)C)c1] | H | H | C |
| 167 | 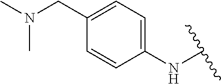 [SMILES: *Nc1ccc(CN(C)C)cc1] | H | 2-F | C |
| 168 | 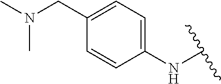 [SMILES: *Nc1ccc(CN(C)C)cc1] | — | H | N |
| 169 | 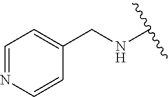 [SMILES: *NCc1ccncc1] | — | H | N |
| 170 | 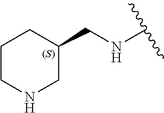 | OH | H | C |
| 171 | 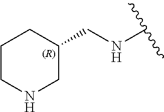 | H | 2-F | C |
| 172 | 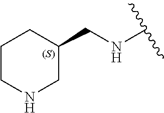 | — | H | N |
| 173 | 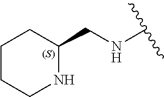 | — | H | N |
| 174 | 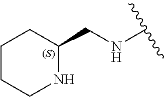 | OH | H | C |
| 175 | 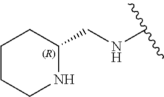 | H | 3-F | C |
| 176 | 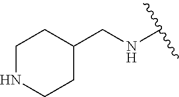 [SMILES: *NCC1CCNCC1] | H | H | C |
| 177 | 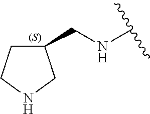 | H | H | C |
| 178 | 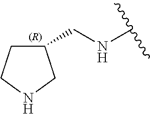 | H | H | C |
| 179 | 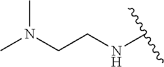 [SMILES: *NCCN(C)C] | H | 3-F | C |
| 180 | 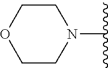 [SMILES: *N1CCOCC1] | — | H | N |
| 181 | 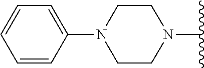 [SMILES: *N1CCN(c2ccccc2)CC1] | — | 2-F | N |
| 182 | 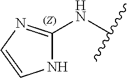 | OH | H | C |
| 183 | 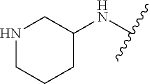 [SMILES: *NC1CCCNC1] | H | H | C |
| 184 | 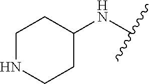 [SMILES: *NC1CCNCC1] | — | H | N |
| 185 | 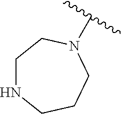 [SMILES: *N1CCCNCC1] | H | H | C |
| 186 | 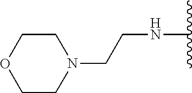 [SMILES: *NCCN1CCOCC1] | H | H | C |
| 187 | 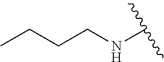 [SMILES: *NCCCC] | OH | H | C |
| 188 | 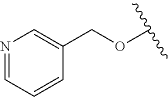 [SMILES: *OCc1cccnc1] | H | H | C |
| 189 | 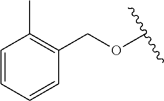 [SMILES: *OCc1ccccc1C] | H | H | C |
| 190 | 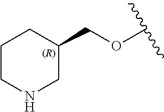 | H | H | C |
| 191 | 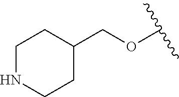 [SMILES: *OCC1CCNCC1] | H | 2-Cl | C |
| 192 | 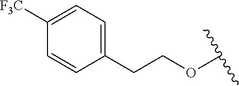 [SMILES: *OCCc1ccc(C(F)(F)F)cc1] | H | H | C |
| 193 | 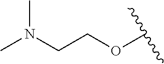 [SMILES: *OCCN(C)C] | — | H | N |
| 194 | 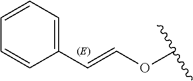 | — | H | N |
| 195 | 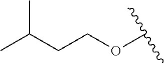 [SMILES: *OCCC(C)C] | — | H | N |

Similarly, using commercially available compounds and largely the procedures set forth in Examples 140-150 and substituting the appropriate starting materials, the compounds 196-240 of Table 6 can be made.

Thus, in some embodiments, provided herein are compounds of the following Formula:

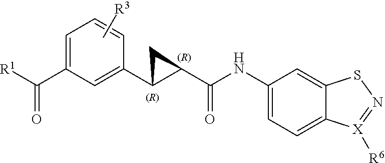

or a pharmaceutically acceptable salt thereof

wherein

R¹ is OH, NH₂, OMe,

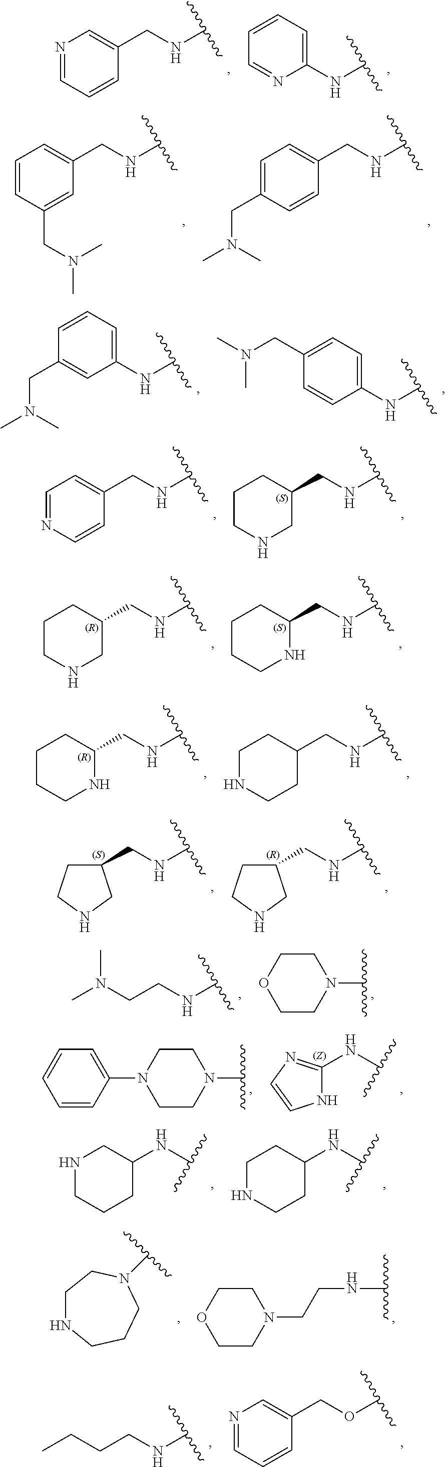

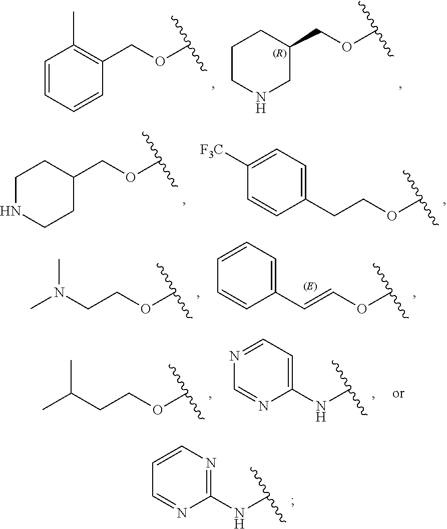

R³ is H, 3-F, 2-F, or 2-Cl; and

X is N and R⁶ is null, or X is C and R⁶ is H, OMe, or OH.

TABLE 6

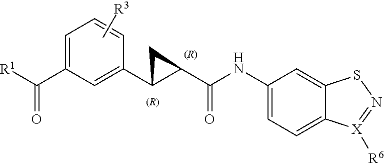

| Example | R¹ | R⁶ | R³ | X |
|---|---|---|---|---|
| 196 | OH | H | H | C |
| 197 | OH | — | 3-F | N |
| 198 | NH₂ | OH | H | C |
| 199 | NH₂ | H | H | C |
| 200 | OMe | — | 2-F | N |
| 201 | OMe | H | H | C |
| 202 | 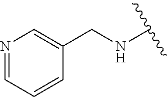 [SMILES: *NCc1cccnc1] | OH | H | C |
| 203 | 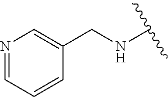 [SMILES: *NCc1cccnc1] | — | H | N |
| 204 | 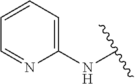 [SMILES: *Nc1ccccn1] | OH | 2-C1 | C |
| 204b | 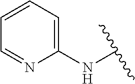 [SMILES: *Nc1ccccn1] | OMe | H | C |
| 205 | 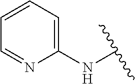 [SMILES: *Nc1ccccn1] | — | H | N |
| 206 | 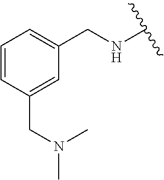 [SMILES: *NCc1cccc(CN(C)C)c1] | H | H | C |
| 207 | 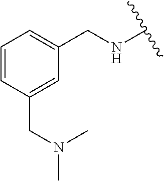 [SMILES: *NCc1cccc(CN(C)C)c1] | — | 3-F | N |
| 208 | 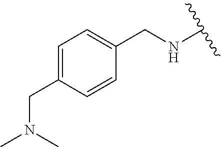 [SMILES: *NCc1ccc(CN(C)C)cc1] | H | H | C |
| 209 | 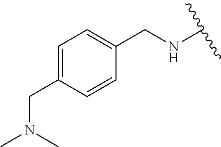 [SMILES: *NCc1ccc(CN(C)C)cc1] | H | H | C |
| 210 | 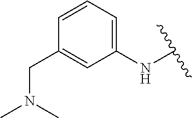 [SMILES: *Nc1cccc(CN(C)C)c1] | — | H | N |
| 211 | 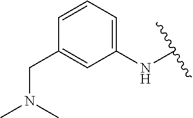 [SMILES: *Nc1cccc(CN(C)C)c1] | H | H | C |
| 212 | 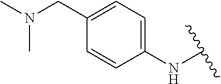 [SMILES: *Nc1ccc(CN(C)C)cc1] | H | 2-F | C |
| 213 | 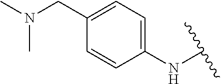 [SMILES: *Nc1ccc(CN(C)C)cc1] | — | H | N |
| 214 | 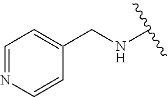 [SMILES: *NCc1ccncc1] | — | H | N |
| 215 | 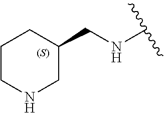 | OH | H | C |
| 216 | 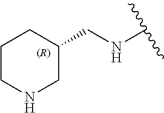 | H | 2-F | C |
| 217 | 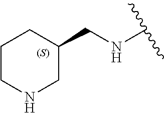 | — | H | N |
| 218 | 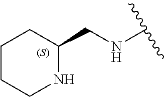 | — | H | N |
| 219 | 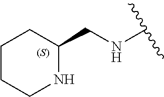 | OH | H | C |
| 220 | 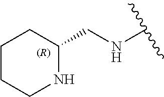 | H | 3-F | C |
| 221 | 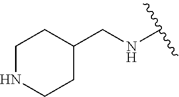 [SMILES: *NCC1CCNCC1] | H | H | C |
| 222 | 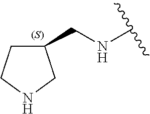 | H | H | C |
| 223 | 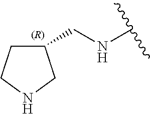 | H | H | C |
| 224 | 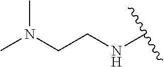 [SMILES: *NCCN(C)C] | H | 3-F | C |
| 225 | 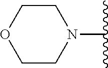 [SMILES: *N1CCOCC1] | — | H | N |
| 226 | 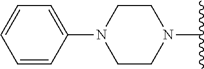 [SMILES: *N1CCN(c2ccccc2)CC1] | — | 2-F | N |
| 227 | 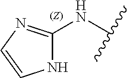 | OH | H | C |
| 228 | 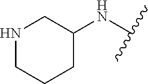 [SMILES: *NC1CCCNC1] | H | H | C |
| 229 | 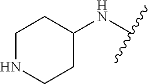 [SMILES: *NC1CCNCC1] | — | H | N |
| 230 | 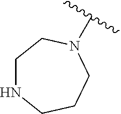 [SMILES: *N1CCCNCC1] | H | H | C |
| 231 | 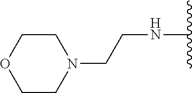 [SMILES: *NCCN1CCOCC1] | H | H | C |
| 232 | 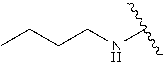 [SMILES: *NCCCC] | OH | H | C |
| 233 | 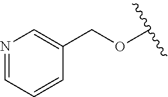 [SMILES: *OCc1cccnc1] | H | H | C |
| 234 | 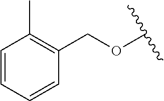 [SMILES: *OCc1ccccc1C] | H | H | C |
| 235 | 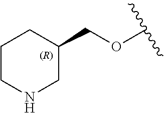 | H | H | C |
| 236 | 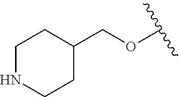 [SMILES: *OCC1CCNCC1] | H | 2-Cl | C |
| 237 | 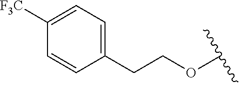 [SMILES: *OCCc1ccc(C(F)(F)F)cc1] | H | H | C |
| 238 | 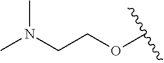 [SMILES: *OCCN(C)C] | — | H | N |
| 239 | 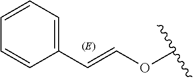 | — | H | N |
| 240 | 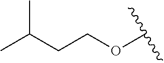 [SMILES: *OCCC(C)C] | — | H | N |
| 241 | 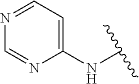 [SMILES: *Nc1ccncn1] | H | H | C |
| 242 | 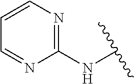 [SMILES: *Nc1ncccn1] | — | H | N |

### Example 246

Topical pharmaceutical compositions for lowering intraocular pressure are prepared by conventional methods and formulated as follows:

| Ingredient | Amount (wt %) |
|---|---|
| Benzoisothiazole amide | 0.50 |
| Dextran 70 | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 5.5-6.5 |
| Purified water | q.s. to 100% |

A compound according to this disclosure is used as the 6-aminobenzoisothiazole derivative. When the composition is topically administered to the eyes once daily, the above composition decreases intraocular pressure in a patient suffering from glaucoma.

### Example 247

Example 246 is repeated using 3-amino-N-(benzoisothiazol-6-yl)-3-(thiophen-3-yl)propanamide dihydrochloride (E6) according to this disclosure. When administered as a drop 2 times per day, the above composition substantially decreases intraocular pressure and serves as a neuroprotective agent.

### Example 248

Example 246 is repeated using a gamma amino acid benzoisothiazole amide according to this disclosure. When administered as a drop twice per day, the above composition substantially decreases intraocular pressure.

### Example 249

Example 246 is repeated using a benzoisothiadiazole according to this disclosure. When administered as a drop twice per day, the above composition substantially decreases allergic symptoms and relieves dry eye syndrome.

### Example 250

Example 246 is repeated using 3-(dimethylamino)-N-(benzoisothiazol-6-yl)-2-(thiophen-3-yl)propanamide dihydrochloride (E44) according to this disclosure. When administered as a drop as needed, the above composition substantially decreases hyperemia, redness and ocular irritation.

### Example 251

Example 246 is repeated using 3-amino-N-(7-fluoro-benzoisothiazol-6-yl)-2-(thiophen-3-yl)propanamide dihydrochloride according to this disclosure. When administered as a drop 4 times per day, the above composition substantially decreases intraocular pressure and serves as a neuroprotective agent.

### Example 252

Example 246 is repeated using 3-amino-N-(benzoisothiazol-6-yl)-2-(thiophen-3-yl)propanamide dihydrochloride (E49) according to this disclosure. When administered as a drop twice per day, the above composition substantially decreases intraocular pressure.

### Example 253

Example 246 is repeated using 4-((1S,2S)-2-(benzoisothiazol-6-ylcarbamoyl)cyclopropyl)-N-(pyridin-2-ylmethyl)benzamide (E150) according to this disclosure. When administered as a drop twice per day, the above composition substantially decreases ocular pressure, allergic symptoms and relieves dry eye syndrome.

Reference Example One. The cell-based porcine trabecular meshwork (PTM) assay.

The anterior section of porcine eyes was harvested within 4 hours post-mortem. The iris and ciliary body were removed and trabecular meshwork cells were harvested by blunt dissection. Finely minced trabecular meshwork tissue was plated into collagen-coated 6-well plates in Medium-199 containing 20% fetal bovine serum (FBS). After two passages at confluence, cells were transferred to low-glucose DMEM containing 10% FBS. Cells were used between passage 3 and passage 8.

Cells were plated into fibronectin-coated, glass multiwell plates the day before compound testing under standard culture conditions. Compounds were added to cells in the presence of 1% FBS-containing DMEM and 1% DMSO. When compounds were incubated with the cells for the duration determined to be optimal, the media and compound is removed and cells fixed for 20 minutes in 3% methanol-free paraformaldehyde. Cells were rinsed twice with phosphate buffered saline (PBS) and cells are permeabilized with 0.5% Triton X-100 for two minutes. Following an additional two washes with PBS, F-actin was stained with Alexa-fluor 488-labelled phalloidin and nuclei are stained with DAPI.

Data was reduced to the mean straight actin-fiber length and normalized to DMSO-treated control cells (100%) and 50 μM Y-27632 (0%). Y-27632 is a rho-kinase inhibitor known to result in the depolymerization of F-actin in these cells.

Reference Example Two. Pharmacological Activity for Glaucoma Assay.

Pharmacological activity for glaucoma can be demonstrated using assays designed to test the ability of the subject compounds to decrease intraocular pressure. Examples of such assays are described in the following reference, incorporated herein by reference: C. Liljebris, G. Selen, B. Resul, J. Stjernschantz, and U. Hacksell, “Derivatives of 17-phenyl-18,19,20-trinorprostaglandin F_{2α} Isopropyl Ester: Potential Anti-glaucoma Agents”, Journal of Medicinal Chemistry 1995, 38 (2): 289-304.

Table 7 provides PKA, PKCη, IKKβ, JAK2, JAK3, ROCK1, or ROCK2 IC₅₀ or Kᵢ values for certain of the compounds provided herein.

TABLE 7

| Example | Structure | IC₅₀ or Kᵢ |
|---|---|---|
| 254 (S)-3-amino-N- (benzo[d]isothiazol-6-yl)- 2-phenylpropanamide | 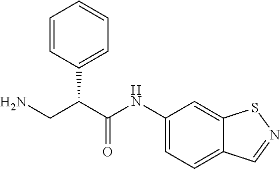 [SMILES: NC[C@@H](C(=O)Nc1ccc2cnsc2c1)c1ccccc1] | <100 nM ROCK1 <100 nM ROCK2 |
| 255 tert-butyl (S)-(3- (benzo[d]isothiazol-6- ylamino)-3-oxo-1- phenylpropyl)carbamate | 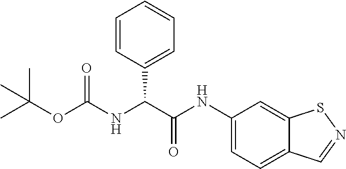 [SMILES: CC(C)(C)OC(=O)N[C@@H](C(=O)Nc1ccc2cnsc2c1)c1ccccc1] | <10 μM ROCK1 <10 μM ROCK2 |
| 256 tert-butyl (S)-(3- (benzo[d]isothiazol-6- ylamino)-3-oxo-2- phenylpropyl)carbamate | 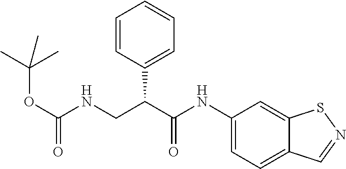 [SMILES: CC(C)(C)OC(=O)NC[C@@H](C(=O)Nc1ccc2cnsc2c1)c1ccccc1] | <10 μM ROCK1 <10 μM ROCK2 |
| 257 N-(benzo[d]isothiazol-6- yl)-2-(dimethylamino)-2- (thiophen-3-yl)acetamide | 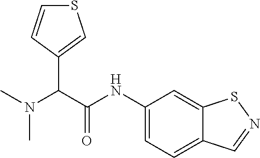 [SMILES: CN(C)C(C(=O)Nc1ccc2cnsc2c1)c1ccsc1] | <1 μM ROCK1 <1 μM ROCK2 |
| 258 (rel)-(1R,2R)-N- (isoquinolin-6-yl)-2-(4- (N-(pyridin-3- ylmethyl)sulfamoyl) phenyl)methyl 4-((1R,2R)-2- (benzo[d]isothiazol-6-yl- carbamoyl)cyclopropyl) benzoate | 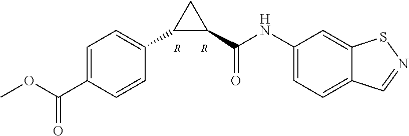 | <1 μM ROCK1 <100 nM ROCK2 |
| 259 (S)-3-amino-n- (benzo[d]isothiazol-6-yl)- 3-phenylproapnamide | 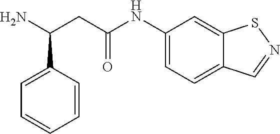 [SMILES: N[C@@H](CC(=O)Nc1ccc2cnsc2c1)c1ccccc1] | <1 μM ROCK1 <1 μM ROCK2 |
| 260 4-((1R,2R)-2- (benzo[d]isothiazol-6- ylcarbamoyl)cyclopropyl)- N-(pyridin-3- ylmethyl)benzamide | 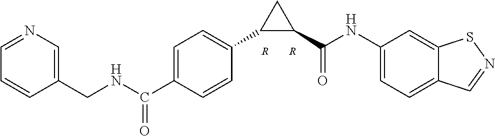 | <10 nM ROCK1 <10 nM ROCK2 <500 nM IKKβ |
| 261 4-((1R,2R)-2- (benzo[d]isothiazol-6- ylcarbamoyl)cyclopropyl)- N-(pyridin-2- ylmethyl)benzamide | 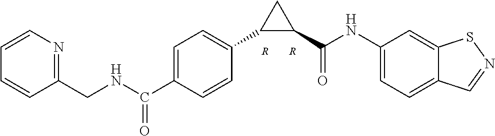 | <10 μM ROCK1 <10 μM ROCK2 <500 nM IKKβ <100 nM JAK3 |
| 262 4-((1R,2R)-2- (benzo[d]isothiazol-6- ylcarbamoyl)cyclopropyl) benzoic acid | 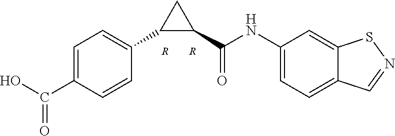 | <500 nM ROCK1 <500 nM ROCK2 |
| 264 | 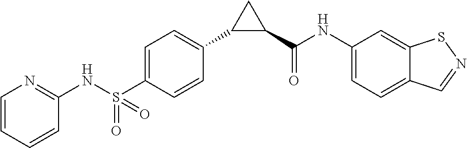 | |
| 265 | 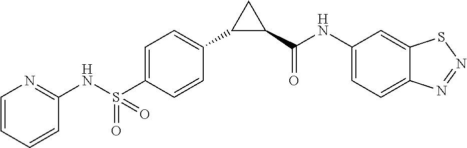 | |
| 266 (rel) (1R,2R)-N- (benzo[d]isothiazol-6-yl)- 2-(4-(N-(pyridin-2- yl)sulfamoyl) phenyl)cyclopropane-1- carboxamide | 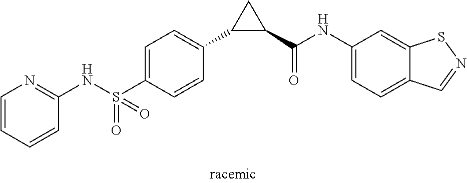 | <10 nM JAK2 <100 nM JAK3 <10 nM ROCK2 <10 nM ROCK1 <15 nM 1KKβ |
| 267 (rel) (1R,2R)-N- (benzo[d] [1,2,3]thiadiazol-6-yl)-2- (4-(N-(pyridin-2- yl)sulfamoyl) phenyl)cyclopropane-1- carboxamide | 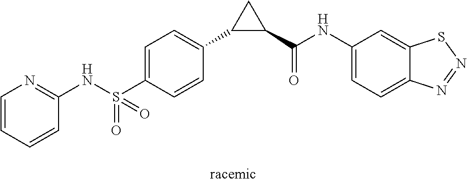 | <100 nM JAK2 <600 nM JAK3 <500 nM ROCK2 <500 nM ROCK1 |
| 268 3-((1r,2r)-2- (benzo[d][1,2,3] thiadiazol-6- ylcarbamoyl)cyclopropyl)- n-(pyridin-2- ylmethyl)benzamide | 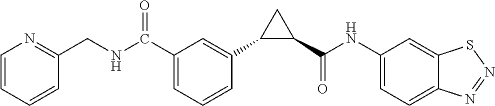 [SMILES: O=C(NCc1ccccn1)c1cccc([C@@H]2C[C@H]2C(=O)Nc2ccc3nnsc3c2)c1] | <500 nM ROCK2 <500 nM ROCK1 |
| 203 (3-((1R,2R)-2- (benzo[d][1,2,3] thiadiazol-6- ylcarbamoyl) cyclopropyl)-N-(pyridin- 3-ylmethyl) benzamide | 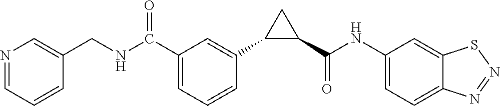 [SMILES: O=C(NCc1cccnc1)c1cccc([C@@H]2C[C@H]2C(=O)Nc2ccc3nnsc3c2)c1] | <600 nM ROCK2 <500 nM ROCK1 |
| 269 n-(benzo[d]isothiazol-6- yl)-2-(4-(N-(pyrimidin-2- yl) sulfamoyl) phenyl) cyclopropane-1- carboxamide | 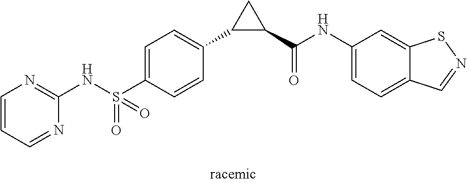 | <10 nM JAK2 <50 nM JAK3 <500 nM ROCK2 <500 nM ROCK1 <50 nM IKKβ |
| 270 (4-((1r,2r)-2- (benzo[d][1,2,3] thiadiazol-6- ylcarbamoyl) cyclopropyl)-N-(pyridin- 2-ylmethyl) benzamide | 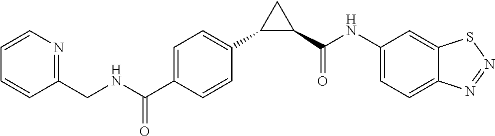 [SMILES: O=C(NCc1ccccn1)c1ccc([C@@H]2C[C@H]2C(=O)Nc2ccc3nnsc3c2)cc1] | <500 nM ROCK2 <500 nM ROCK1 <500 nM IKKβ |
| 158 | 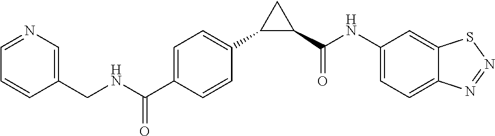 [SMILES: O=C(NCc1cccnc1)c1ccc([C@@H]2C[C@H]2C(=O)Nc2ccc3nnsc3c2)cc1] | <500 nM ROCK2 <500 nM ROCK1 <10 μM IKKβ |
| 271 | 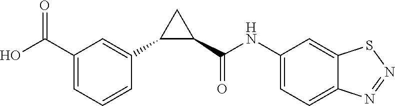 [SMILES: O=C(O)c1cccc([C@@H]2C[C@H]2C(=O)Nc2ccc3nnsc3c2)c1] | <10 μM ROCK2 <10 μM ROCK1 |
| 272 | 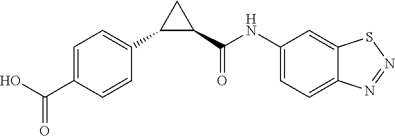 [SMILES: O=C(O)c1ccc([C@@H]2C[C@H]2C(=O)Nc2ccc3nnsc3c2)cc1] | <50 μM ROCK2 <50 μM ROCK1 |
| 273 trans-N- (benzo[d]isothiazol-6-yl)- 2-phenylcyclopropane-1- carboxamide | 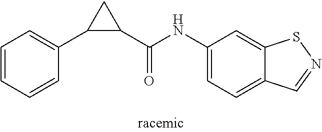 [SMILES: O=C(Nc1ccc2cnsc2c1)C1CC1c1ccccc1] | <50 nM ROCK2 <50 nM ROCK1 <10 μM nM PKA |
| 274 (1r,2r)-n- (benzo[d]isothiazol-6-yl)- 2-phenylcyclopropane-1- carboxamide | 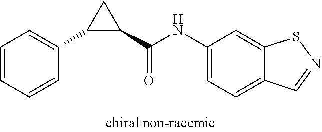 | <50 nM ROCK2 <50 nM ROCK1 <10 μM PKA |
| 275 (1r,2r)-n- (benzo[d]isothiazol-6-yl)- 2-phenylcyclopropane-1- carboxamide | 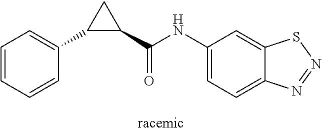 | <10 μM ROCK2 <10 μM ROCK1 |
| 276 (1r,2r)-n- (benzo[d]isothiazol-6-yl)- 2-phenylcyclopropane-1- carboxamide | 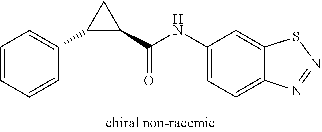 | <10 μM ROCK2 <10 μM ROCK1 |
| 277 | 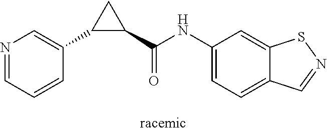 | <1 μM ROCK2 <1 μM ROCK1 |
| 278 | 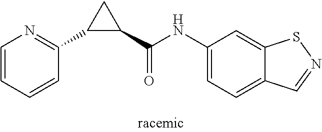 | <500 nM ROCK2 <500 nM ROCK1 |
| 279 | 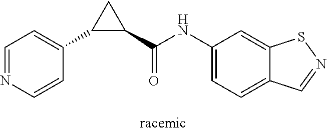 | <500 nM ROCK2 <500 nM ROCK1 |
| 280 (1r,2r)-n- (benzo[d]isothiazol-6-yl)- 2-phenylcycloproopane-1- carboxamide | 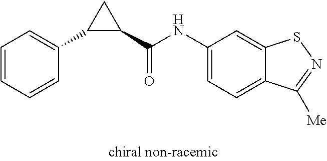 | |
| 281 (1r,2r)-n- (benzo[d]isothiazol-6-yl)- 2-phenylcyclopropane-1- carboxamide | 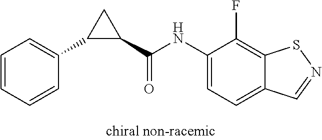 | |

While the disclosure has been described in detail and with reference to specific embodiments thereof, it will be apparent to one skilled in the art that various changes and modifications can be made without departing from the spirit and scope of the disclosure.

What is claimed is:

1. A compound of formula (I): 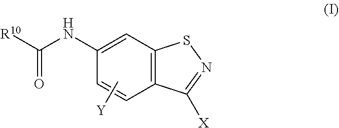
or any optical isomer, diastereomer, enantiomer, tautomer, physiologically acceptable salt, or physiologically acceptable solvate thereof;
wherein R¹⁰ is, C₁-C₈ alkyl, C₂-C₈ alkenyl, C₂-C₈ alkynyl, C₁-C₈ carbonyl, C₁-C₈ carbonylamino, C₁-C₈ alkoxy, C₁-C₈ sulfonyl, C₁-C₈ sulfonylamino, C₁-C₈ thioalkyl or C₁-C₈ carboxyl, an alkylaryl group, an aryl group, an alkylheteroaryl group, a heteroaryl group, a and alkylcycloalkyl group, a cycloalkyl group, an alkylheterocycloalkyl group, a heterocycloalkyl group, each existent stereocenter being either ‘R’ or ‘S’ in configuration independently; and
X and Y are, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

2. The compound according to claim 1, wherein R¹⁰ is an alkylaryl group, an aryl group, an alkylheteroaryl group, a heteroaryl group, a and alkylcycloalkyl group, a cycloalkyl group, an alkylheterocycloalkyl group, a heterocycloalkyl group, each existent stereocenter being either ‘R’ or ‘S’ in configuration independently.

3. The compound according to claim 2, wherein R¹⁰, together with the atoms to which it is attached, forms an alpha amino acid, a beta amino acid, or a gamma amino acid.

4. The compound according to claim 1, wherein X and Y are, independently, hydrogen, hydroxyl, halogen, amino, cyano, C₁-C₄ carbonyl, C₁-C₄ carbonylamino, C₁-C₄ alkoxy.

5. A compound of formula (II): 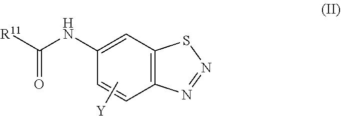
or any optical isomer, diastereomer, enantiomer, tautomer, physiologically acceptable salt, or physiologically acceptable solvate thereof;
wherein R¹¹ is, C₁-C₈ alkyl, C₂-C₈ alkenyl, C₂-C₈ alkynyl, C₁-C₈ carbonyl, C₁-C₈ carbonylamino, C₁-C₈ alkoxy, C₁-C₈ sulfonyl, C₁-C₈ sulfonylamino, C₁-C₈ thioalkyl or C₁-C₈ carboxyl, an alkylaryl group, an aryl group, an alkylheteroaryl group, a heteroaryl group, a and alkylcycloalkyl group, a cycloalkyl group, an alkylheterocycloalkyl group, a heterocycloalkyl group, each existent stereocenter being either ‘R’ or ‘S’ in configuration independently; and
Y is hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

6. The compound according to claim 5, wherein R¹¹ is an alkylaryl group, an aryl group, an alkylheteroaryl group, a heteroaryl group, a and alkylcycloalkyl group, a cycloalkyl group, an alkylheterocycloalkyl group, a heterocycloalkyl group, each existent stereocenter being either ‘R’ or ‘S’ in configuration independently.

7. The compound according to claim 6, wherein R¹¹, together with the atoms to which it is attached, forms an alpha amino acid, a beta amino acid, or a gamma amino acid.

8. The compound according to claim 5, wherein Y is, hydrogen, hydroxyl, halogen, amino, cyano, C₃-C₆ cycloalkyl, C₁-C₄ carbonyl, C₁-C₄ carbonylamino, C₁-C₄ alkoxy.

9. A compound of Formula (III): 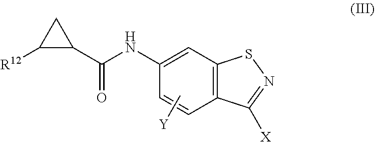
or any optical isomer, diastereomer, enantiomer, tautomer, physiologically acceptable salt, or physiologically acceptable solvate thereof;
wherein R¹² is hydrogen, C₁-C₄ alkyl, C₂-C₄ alkenyl, C₂-C₄ alkynyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl, an aryl group, a heteroaryl group, a cycloalkyl group, a heterocycloalkyl group, C₁-C₈ alkyl, C₂-C₈ alkenyl, or C₂-C₈ alkynyl, the stereocenters being either ‘R’ or ‘S’ in configuration independently; and
X and Y are, independently, hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

10. The compound according to claim 9, wherein R¹² is an alkylaryl group, an aryl group, an alkylheteroaryl group, a heteroaryl group, a and alkylcycloalkyl group, a cycloalkyl group, an alkylheterocycloalkyl group, a heterocycloalkyl group, each existent stereocenter being either ‘R’ or ‘S’ in configuration independently.

11. The compound according to claim 9, wherein R¹² is minimally a meta- or a para-substituted aryl group, or a heteroaryl group.

12. The compound according to claim 9, wherein X and Y are, independently, hydrogen, hydroxyl, halogen, amino, cyano, C₃-C₆ cycloalkyl, C₁-C₄ carbonyl, C₁-C₄ carbonylamino, C₁-C₄ alkoxy.

13. A compound of Formula (IV): 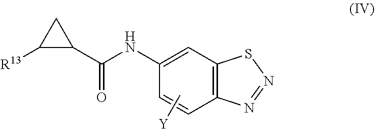
or any optical isomer, diastereomer, enantiomer, tautomer, physiologically acceptable salt, or physiologically acceptable solvate thereof;
wherein R¹³ is hydrogen, C₁-C₄ alkyl, C₂-C₄ alkenyl, C₂-C₄ alkynyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl, an aryl group, a heteroaryl group, a cycloalkyl group, a heterocycloalkyl group, C₁-C₈ alkyl, C₂-C₈ alkenyl, or C₂-C₈ alkynyl, the stereocenters being either ‘R’ or ‘S’ in configuration independently; and
Y is hydrogen, hydroxyl, halogen, C₁-C₄ alkyl, C₂-C₄ alkenyl, C₂-C₄ alkynyl, amino, nitro, cyano, C₃-C₆ cycloalkyl, C₁-C₄ carbonyl, C₁-C₄ carbonylamino, C₁-C₄ alkoxy, C₁-C₄ sulfonyl, C₁-C₄ sulfonylamino, C₁-C₄ thioalkyl or C₁-C₄ carboxyl.

14. The compound according to claim 13, wherein R¹³ is an alkylaryl group, an aryl group, an alkylheteroaryl group, a heteroaryl group, a and alkylcycloalkyl group, a cycloalkyl group, an alkylheterocycloalkyl group, a heterocycloalkyl group, the stereocenters being either ‘R’ or ‘S’ in configuration independently.

15. The compound according to claim 13, wherein R¹³ is minimally a meta- or a para-substituted aryl group, or a heteroaryl group.

16. The compound according to claim 13, wherein Y is hydrogen, hydroxyl, halogen, amino, cyano, C₁-C₄ carbonyl, C₁-C₄ carbonylamino, or C₁-C₄ alkoxy.

17. The compound according to claim 13, wherein Y is hydroxyl, halogen, amino, or cyano.

18. A compound of Formula (V): 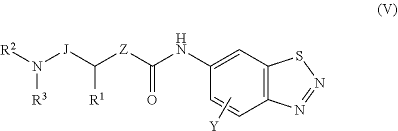
or a pharmaceutically acceptable salt thereof;
wherein
X is C—R⁶ or N;
J is a bond, methylene or ethylene;
Z is a bond, methylene or ethylene;
R¹ is hydrogen; halogen; —C₁₋₆-alkyl; —C₁₋₆-haloalkyl, —(C₁₋₆-alkyl)-OH; —C₆₋₁₀-aryl; heteroaryl; —CH₂-heteroaryl; —CH₂—(C₆₋₁₀-aryl); —C₃₋₁₀-cycloalkyl; —CH₂—(C₃₋₁₀-cycloalkyl); —C(O)N(H)—(C₆₋₁₀-aryl); —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl) substituted with halogen, —C₁₋₆-alkyl, —C₁₋₆-haloalkyl, or —(C₁₋₆-alkyl)-OH; 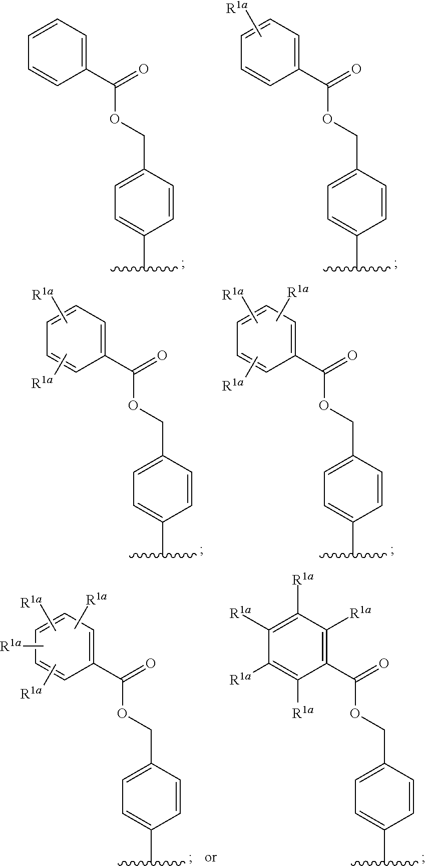
R^{1a} is, independently, halogen, —C₁₋₆-alkyl, or —C₁₋₆-haloalkyl;
R² is hydrogen, —C₁₋₆-alkyl, —CH₂—(C₆₋₁₀-aryl), —(C₁₋₆-alkyl)N(C₁₋₆-alkyl)C₁₋₆-alkyl, or —C(NH)NH₂;
R³ is hydrogen or —C₁₋₆-alkyl;
or R² and R³, together with the atoms to which they are attached, form a C₂₋₆-heterocycloalkyl;
or Z is CH and R³ and Z, together with the atoms to which they are attached, form a C₃₋₆-heterocycloalkyl;
R⁶ is hydrogen, —C₁₋₆-alkyl, —OH, —CN, —O—(C₁₋₆-alkyl), —C(H)(F)—CH₃, or —C₁₋₆-haloalkyl.

19. The compound of claim 18, wherein the compound of Formula (V) is a compound of Formula (V1): 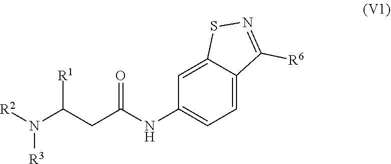
or a pharmaceutically acceptable salt thereof;
wherein
R¹ is —C₁₋₆-alkyl; —C₆₋₁₀-aryl; —C₆₋₁₀-aryl monosubstituted with halogen, —C₁₋₆-alkyl or hydroxymethyl; —C₆₋₁₀-aryl disubstituted with halogen, —C₁₋₆-alkyl or hydroxymethyl; heteroaryl; —CH₂-heteroaryl; —CH₂—(C₆₋₁₀-aryl); —CH₂—(C₆₋₁₀-aryl) monosubstituted with halogen; —C₃₋₁₀-cycloalkyl; or —CH₂—(C₃₋₁₀-cycloalkyl);
R² is hydrogen or —C₁₋₆-alkyl;
R³ is hydrogen or —C₁₋₆-alkyl; and
R⁶ is hydrogen or —OH.

20. The compound of claim 18, wherein R² is hydrogen or methyl.

21. The compound of claim 18, wherein R³ is hydrogen or methyl.

22. The compound of claim 18, wherein R¹ is methyl; phenyl; phenyl monosubstituted with halogen, methyl or hydroxymethyl; phenyl disubstituted with halogen, methyl or hydroxymethyl; thienyl; —CH₂-thienyl; furyl; pyridyl; benzyl; benzyl monosubstituted with halogen; cyclohexyl; cyclopropyl; —CH₂-cyclohexyl; thiazole; oxazole; or piperidyl.

23. The compound of claim 18, wherein the compound of Formula (V) is a compound of Formula (V2): 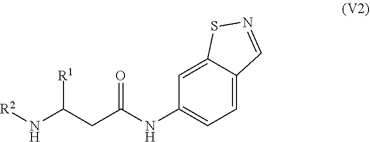
or a pharmaceutically acceptable salt thereof.

24. The compound of claim 18, wherein R² is hydrogen.

25. The compound of claim 18, wherein the compound is:

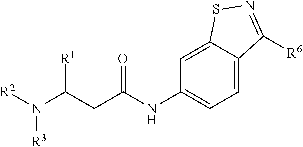

| R⁶ | R¹ | R² | R³ |
|---|---|---|---|
| H | (S)-C₆H₅ | H | H |
| H | (R)-C₆H₅ | H | H |
| OH | (S)-C₆H₅ | H | H |
| OH | (R)-C₆H₅ | H | H |
| OH | (S)-C₆H₅ | Me | Me |
| H | (S)-C₆H₅ | Me | H |
| H | (±)-o-chloro-C₆H₄ | H | H |
| H | (±)-p-chloro-C₆H₄ | H | H |
| H | (±)-p-hydroxymethyl- | H | H |
| | C₆H₄ | | |
| OH | (±)-p-fluoro-C₆H₄ | H | H |
| H | (±)-p-fluoro-C₆H₄ | Me | H |
| H | (S)-3-thienyl | H | H |
| OH | (S)-3-thienyl | H | H |
| H | (S)-3-thienyl | Me | Me |
| OH | (S)-3-thienyl | Me | Me |
| H | (R)-3-thienyl | Me | Me |
| OH | (S)-CH₂-2-thienyl | Me | Me |
| OH | (S)-3-thienyl | Me | H |
| H | (S)-2-thienyl | Me | Me |
| H | (R)-2-thienyl | Me | Me |
| H | 3-furyl | H | H |
| OH | 2-furyl | Me | Me |
| OH | 3,5-difluoroC₆H₃ | Me | H |
| H | m-CH₃ | H | H |
| H | (S)-3-pyridyl | H | H |
| OH | 4-pyridyl | Me | Me |
| H | Benzyl | H | H |
| H | Cyclohexyl | Me | Me |
| H | Cyclopropyl | H | H |
| OH | Methyl cyclohexyl | Me | H |
| H | 4-fluorobenzyl | H | H |
| H | 2-thiazole | Me | Me |
| OH | 2-oxazole | H | Me |
| H | 3-piperdyl | Me | Me |

or a pharmaceutically acceptable salt thereof.

26. The compound of claim 18, wherein the compound of Formula (V) is a compound of Formula (V3): 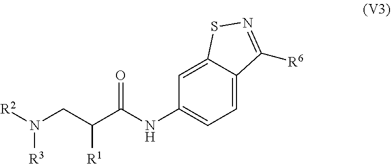
or a pharmaceutically acceptable salt thereof;
wherein
R⁶ is hydrogen, methyl, —OH, —CN, —OCH₃, —C(H)(F)—CH₃, or —CH₂F;
R¹ is —C₆₋₁₀-aryl; —C₆₋₁₀-aryl monosubstituted with halogen, —C₁₋₆-alkyl or hydroxymethyl; heteroaryl; —C₃₋₁₀-cycloalkyl; —C(O)N(H)—(C₆₋₁₀-aryl); 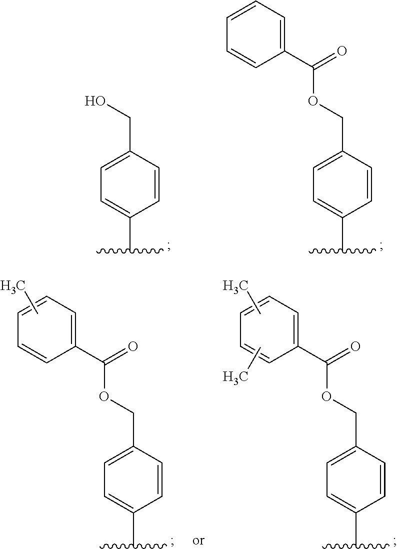
R² is hydrogen or —C₁₋₆-alkyl; and
R³ is hydrogen or —C₁₋₆-alkyl.

27. The compound of claim 26, wherein R⁶ is hydrogen, methyl, —OH, or —CN.

28. The compound of claim 26, wherein R² is hydrogen or methyl.

29. The compound of claim 26, wherein R³ is hydrogen or methyl.

30. The compound of claim 26, wherein R¹ is thienyl; phenyl; phenyl substituted with halogen or methyl; cyclohexyl; benzothiphene; —C(O)N(H)-phenyl;

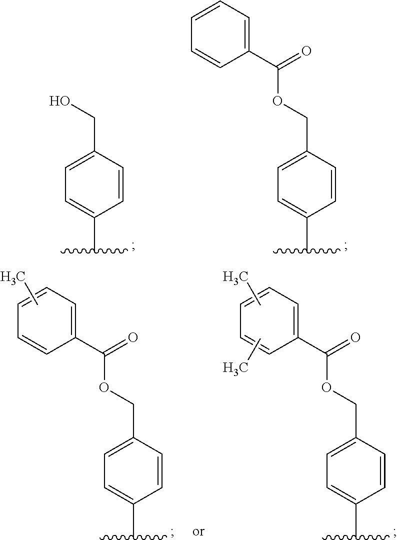

31. The compound of claim 26, wherein the compound of Formula (V3) is a compound of Formula (V3a): 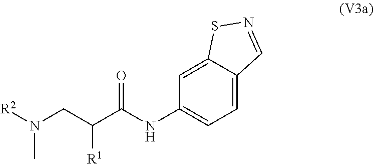
or a pharmaceutically acceptable salt thereof.

32. The compound of claim 26, wherein R² is methyl.

33. The compound of claim 26, wherein the compound is:

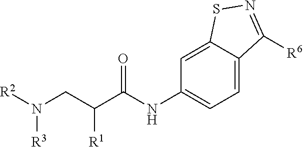

| R⁶ | R¹ | R² | R³ |
|---|---|---|---|
| OH | (±)-3-thienyl | Me | Me |
| H | (±)-3-thienyl | H | H |
| H | C₆H₅ | H | H |
| H | C₆H₅ | Me | Me |
| CN | C₆H₅ | H | H |
| H | 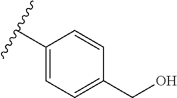 [SMILES: *c1ccc(CO)cc1] | H | H |
| H | 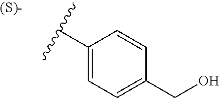 [SMILES: "(S)-" ; *c1ccc(CO)cc1] | H | H |
| H | 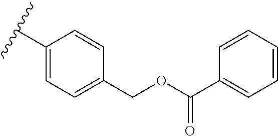 [SMILES: *c1ccc(COC(=O)c2ccccc2)cc1] | H | H |
| H | 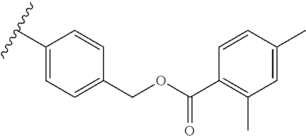 [SMILES: *c1ccc(COC(=O)c2ccc(C)cc2C)cc1] | H | H |
| OMe | 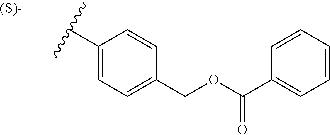 [SMILES: *c1ccc(COC(=O)c2ccccc2)cc1] | H | H |
| CHF—CH₃ | 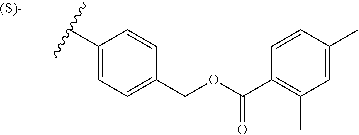 [SMILES: *c1ccc(COC(=O)c2ccc(C)cc2C)cc1] | H | H |
| CH₂F | 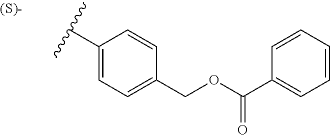 [SMILES: *c1ccc(COC(=O)c2ccccc2)cc1] | Me | Me |
| OH | (±)-2-thienyl | H | H |
| H | (R)-C₆H₅ | H | H |
| CH₃ | (S)-C₆H₅ | H | H |
| H | p-fluoro-C₆H₄ | H | H |
| H | 4-chloro-C₆H₄ | H | H |
| OH | (S)-4-chloro-C₆H₄ | H | H |
| H | 4-methyl-C₆H₄ | H | H |
| H | (S)-4-CH₃—C₆H₄ | H | H |
| OH | cyclohexyl | H | H |
| H | 3-benzo[b]thiophene | Me | Me |
| H | 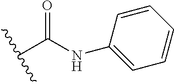 [SMILES: *C(=O)Nc1ccccc1] | H | H |

or a pharmaceutically acceptable salt thereof.

34. The compound of claim 18, wherein the compound of Formula (V) is a compound of Formula (V4): 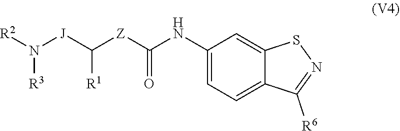
or a pharmaceutically acceptable salt thereof;
wherein
J is a bond, methylene, or ethylene;
Z is a bond, methylene, or ethylene;
R¹ is —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen; C₄₋₈-heteroaryl; 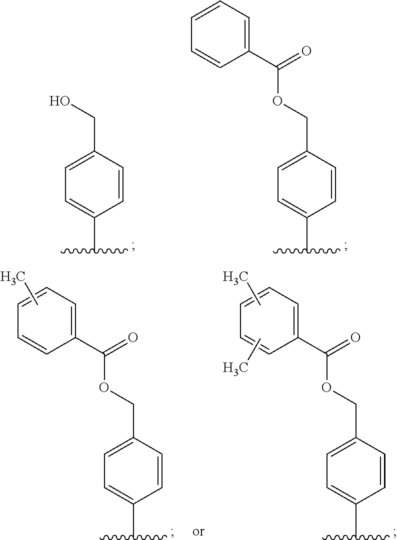
R² is hydrogen, —C₁₋₆-alkyl, —CH₂—(C₆₋₁₀-aryl), or —C(NH)NH₂;
R³ is hydrogen or —C₁₋₆-alkyl;
or Z is CH and R³ and Z, together with the atoms to which they are attached, form a C₃₋₆-heterocycloalkyl; and
R⁶ is hydrogen, —C₁₋₆-alkyl, —OH, —O—(C₁₋₆-alkyl), —CN, or —C₁₋₆-haloalkyl.

35. The compound of claim 34, wherein R⁶ is hydrogen, methyl, —OH, or —CN.

36. The compound of claim 34, wherein R³ is hydrogen or methyl.

37. The compound of claim 34, wherein R² is hydrogen, methyl, or benzyl.

38. The compound of claim 34, wherein R³ is hydrogen and R² is —C(NH)NH₂.

39. The compound of claim 34, wherein R³ is methyl and R² is methyl.

40. The compound of claim 34, wherein R³ is hydrogen and R² is methyl.

41. The compound of claim 34, wherein R¹ is hydrogen; —C₆₋₁₀-aryl; —C₆₋₁₀-aryl substituted with halogen; —C₃₋₁₀-cycloalkyl; or C₄₋₈-heteroaryl.

42. The compound of claim 34, wherein R¹ is hydrogen; —C₆₋₁₀-aryl; —C₆₋₁₀-aryl substituted with halogen, —C₁₋₆-alkyl, or —(C₁₋₆-alkyl)-OH; —CH₂—(C₆₋₁₀-aryl); —CH₂—(C₆₋₁₀-aryl) substituted with halogen; or C₄₋₈-heteroaryl.

43. The compound of claim 34, wherein R¹ is hydrogen; —C₆₋₁₀-aryl; —C₆₋₁₀-aryl substituted with halogen, —C₁₋₆-alkyl or —C₁₋₆-haloalkyl;

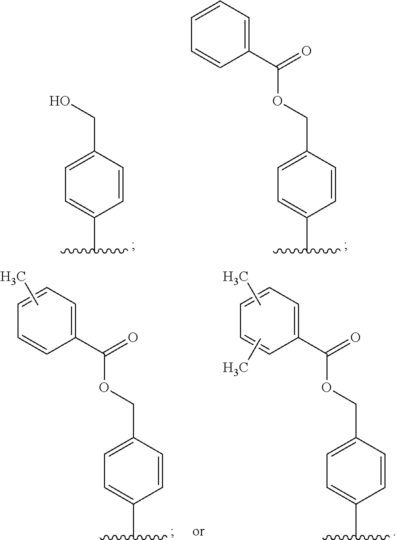

44. The compound of claim 34, wherein the compound of Formula (V4) is a compound of Formula (V4a): 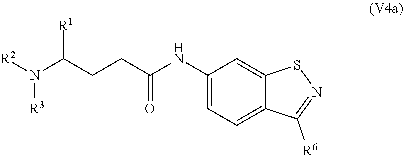
or a pharmaceutically acceptable salt thereof.

45. The compound of claim 34, wherein the compound of Formula (V4) is a compound of Formula (V4b): 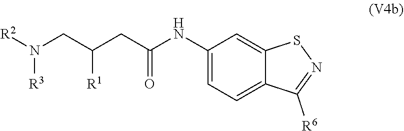
or a pharmaceutically acceptable salt thereof.

46. The compound of claim 34, wherein the compound of Formula (V4) is a compound of Formula (V4c): 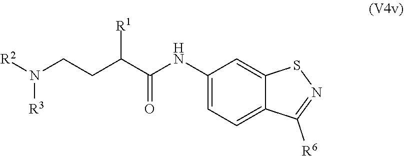
or a pharmaceutically acceptable salt thereof.

47. The compound of claim 34, wherein
J is a bond;
Z is ethylene; and
R¹ is phenyl, phenyl substituted with halogen, cyclopropyl, thienyl, or cyclohexyl.

48. The compound of claim 34, wherein
J is methylene;
Z is methylene; and
R¹ is phenyl, phenyl substituted with halogen, benzyl, benzyl substituted with halogen, or thienyl.

49. The compound of claim 34, wherein
J is ethylene;
Z is a bond; and
R¹ is hydrogen; phenyl; phenyl substituted with halogen, methyl or fluoromethyl;

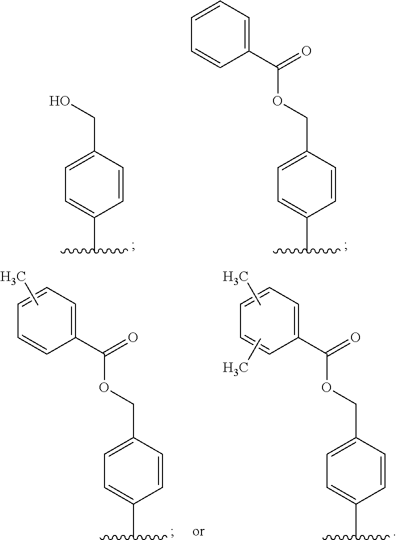

50. The compound of claim 34, wherein Z is CH and R³ and Z, together with the atoms to which they are attached, form a C₃₋₆-heterocycloalkyl.

51. The compound of claim 34, wherein the compound is:

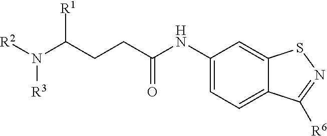

| R⁶ | R¹ | R³ | R² |
|---|---|---|---|
| H | C₆H₅ | H | H |
| OH | C₆H₅ | H | H |
| H | (S)-C₆H₅ | H | H |
| OMe | (R)-C₆H₅ | H | H |
| OEt | (S)-C₆H₅ | H | H |
| H | (S)-C₆H₅ | CH₃ | CH₃ |
| H | 4-F—C₆H₄ | H | H |
| H | 4-Cl—C₆H₄ | CH₃ | H |
| CN | cyclopropyl | CH₃ | H |
| CH₃ | 3-thienyl | H | H |
| H | (S)-3-thienyl | H | H |
| OH | cyclohexyl | CH₃ | CH₃ |
| H | C₆H₅ | CH₂C₆H₅ | H |

or a pharmaceutically acceptable salt thereof.

52. The compound of claim 34, wherein the compound is:

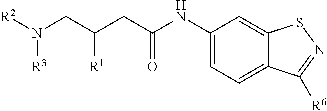

| R⁶ | R¹ | R³ | R² |
|---|---|---|---|
| H | C₆H₅ | H | H |
| CH₂F | C₆H₅ | CH₃ | H |
| CHFCH₃ | 4-chloro-C₆H₄ | H | H |
| OH | CH₂C₆H₅ | CH₃ | H |
| H | 3-thienyl | CH₃ | H |
| OH | 2-thienyl | CH₃ | CH₃ |
| H | p-chloro benzyl | H | H |

or a pharmaceutically acceptable salt thereof.

53. The compound of claim 34, wherein the compound is:

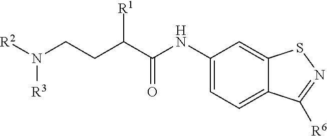

| R⁶ | R¹ | R³ | R² |
|---|---|---|---|
| H | C₆H₅ | H | H |
| H | 4-Cl—C₆H₄ | H | H |
| H | 4-CH₃—C₆H₄ | H | H |
| H | 4-CH₃—C₆H₄ | CH₃ | H |
| H | 4-CF₃—C₆H₄ | H | H |
| H | 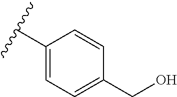 [SMILES: *c1ccc(CO)cc1] | H | H |
| H | 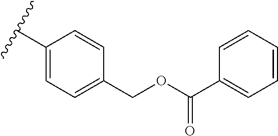 [SMILES: *c1ccc(COC(=O)c2ccccc2)cc1] | H | H |
| H | 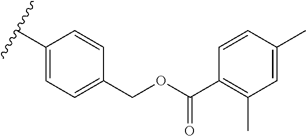 [SMILES: *c1ccc(COC(=O)c2ccc(C)cc2C)cc1] | H | H |

or a pharmaceutically acceptble salt thereof.

54. The compound of claim 34, wherein the compound of Formula (V4) is a compound of Formula (V4d): 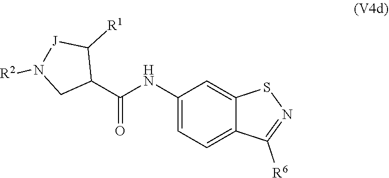
or a pharmaceutically acceptable salt thereof.

55. The compound of claim 34, wherein Z is CH and R³ and Z, together with the atoms to which they are attached, form a pyrrolidinyl.

56. The compound of claim 34, wherein the compound of Formula (V4) is a compound of Formula (V4e): 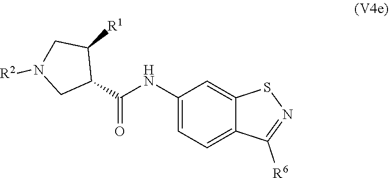
or a pharmaceutically acceptable salt thereof.

57. The compound of claim 34, wherein R¹ is phenyl or phenyl substituted with halogen, methyl, ethyl, or —CH₂OH.

58. A compound of Formula (VI): 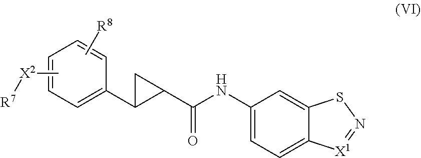
or a pharmaceutically acceptable salt thereof;
wherein
X¹ is C—R⁶ or N;
X² is —C(O)— or —SO₂—;
R⁷ is —OH; —NH₂; —O—(C₁₋₆-alkyl); —N(H)—(C₁₋₃-alkyl)-heteroaryl; —N(H)— heteroaryl; —N(H)—(C₁₋₃-alkyl)-(C₆₋₁₀-aryl)-(C₁₋₃-alkyl)-N(C₁₋₃-alkyl)₂; —N(H)—(C₆₋₁₀-aryl)-(C₁₋₃-alkyl)-N(C₁₋₃-alkyl)₂; —N(H)—(C₁₋₃-alkyl)-heteroalkyl; —N(H)—(C₁₋₃-alkyl)-N(C₁₋₃-alkyl)₂; heteroalkyl; -heteroalkyl-(C₆₋₁₀-aryl); —N(H)-heteroalkyl; heteroalkyl; —N(H)—(C₁₋₆-alkyl); —O—(C₁₋₃-alkyl)-heteroaryl; —O—(C₁₋₃-alkyl)-(C₆₋₁₀-aryl) substituted with —C₁₋₃-alkyl or C₁₋₃-haloalkyl; —O—(C₁₋₃-alkyl)-heteroalkyl; —O—(C₁₋₃-alkyl)-N(C₁₋₃-alkyl)₂; —O—(C₁₋₃-alkyl)-(C₆₋₁₀-aryl); or —O—(C₁₋₆-alkyl);
R⁶ is hydrogen or —OH; and
R⁸ is hydrogen or halogen.

59. The compound of claim 58, wherein X¹ is N.

60. The compound of claim 58, wherein X¹ is C—R⁶.

61. The compound of claim 58, wherein X² is —SO₂—.

62. The compound of claim 58, wherein R⁷ is —OH; —NH₂; —OCH₃; —N(H)CH₂-pyridinyl; —N(H)-pyridinyl; —N(H)CH₂-phenyl-CH₂N(CH₃)₂; —N(H)-phenyl-CH₂N(CH₃)₂; —N(H)CH₂-piperidinyl; —N(H)CH₂-pyrrolidinyl; —N(H)CH₂CH₂N(CH₃)₂; morpholinyl; -piperazyinyl-phenyl; —N(H)-piperidinyl; diazepanyl; —N(H)CH₂CH₂-morpholinyl; —N(H)-butyl; —OCH₂-pyridinyl; —OCH₂-(methylphenyl); —OCH₂-piperidinyl; —OCH₂CH₂-(trifluoromethylphenyl); —OCH₂CH₂N(CH₃)₂; —OCHCH-phenyl; —O-pentanyl; or —N(H)-pyrimidinyl.

63. The compound of claim 58, wherein R⁶ is H.

64. The compound of claim 58, wherein R⁸ is halogen.

65. The compound of claim 58, wherein the compound of Formula (VI) is a compound of (VII): 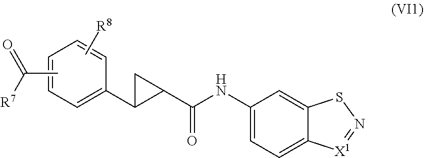
or a pharmaceutically acceptable salt thereof.

66. The compound of claim 58, wherein the compound of Formula (VI) is a compound of Formula (VI2): 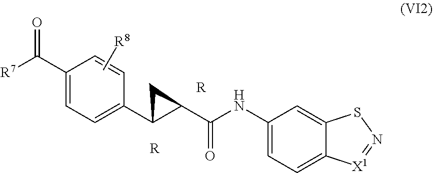
or a pharmaceutically acceptable salt thereof.

67. The compound of claim 58, wherein the compound of Formula (VI) is a compound of Formula (V13): 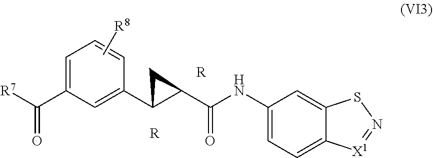
or a pharmaceutically acceptable salt thereof.

68. The compound of claim 58, wherein the compound is:

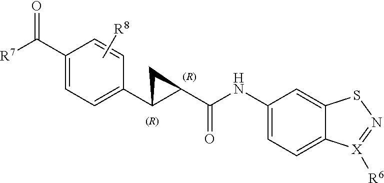

| R⁷ | R⁶ | R⁸ | X |
|---|---|---|---|
| OH | H | H | C |
| OH | — | 3-F | N |
| NH₂ | OH | H | C |
| NH₂ | H | H | C |
| OMe | — | 2-F | N |
| OMe | H | H | C |
| 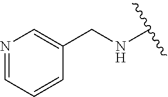 [SMILES: *NCc1cccnc1] | OH | H | C |
| 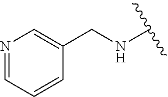 [SMILES: *NCc1cccnc1] | — | H | N |
| 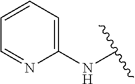 [SMILES: *Nc1ccccn1] | OH | 2-Cl | C |
| 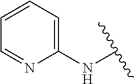 [SMILES: *Nc1ccccn1] | — | H | N |
| 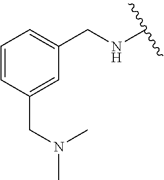 [SMILES: *NCc1cccc(CN(C)C)c1] | H | H | C |
| 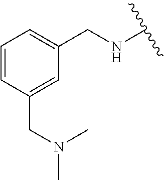 [SMILES: *NCc1cccc(CN(C)C)c1] | — | 3-F | N |
| 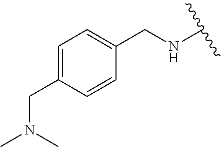 [SMILES: *NCc1ccc(CN(C)C)cc1] | H | H | C |
| 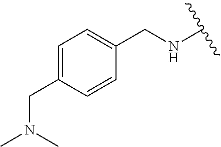 [SMILES: *NCc1ccc(CN(C)C)cc1] | H | H | C |
| 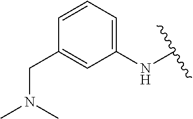 [SMILES: *Nc1cccc(CN(C)C)c1] | — | H | N |
| 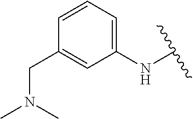 [SMILES: *Nc1cccc(CN(C)C)c1] | H | H | C |
| 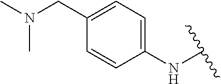 [SMILES: *Nc1ccc(CN(C)C)cc1] | H | 2-F | C |
| 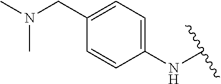 [SMILES: *Nc1ccc(CN(C)C)cc1] | — | H | N |
| 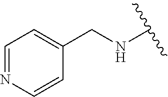 [SMILES: *NCc1ccncc1] | — | H | N |
| 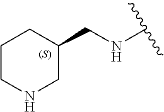 | OH | H | C |
| 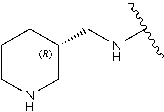 | H | 2-F | C |
| 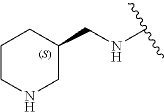 | — | H | N |
| 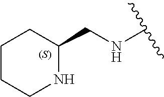 | — | H | N |
| 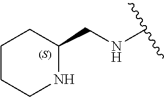 | OH | H | C |
| 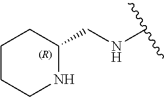 | H | 3-F | C |
| 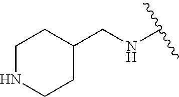 [SMILES: *NCC1CCNCC1] | H | H | C |
| 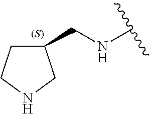 | H | H | C |
| 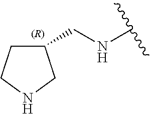 | H | H | C |
| 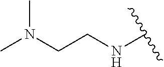 [SMILES: *NCCN(C)C] | H | 3-F | C |
| 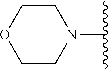 [SMILES: *N1CCOCC1] | — | H | N |
| 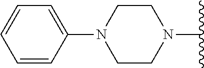 [SMILES: *N1CCN(c2ccccc2)CC1] | — | 2-F | N |
| 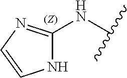 | OH | H | C |
| 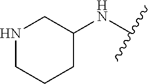 [SMILES: *NC1CCCNC1] | H | H | C |
| 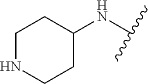 [SMILES: *NC1CCNCC1] | — | H | N |
| 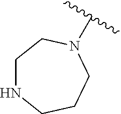 [SMILES: *N1CCCNCC1] | H | H | C |
| 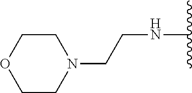 [SMILES: *NCCN1CCOCC1] | H | H | C |
| 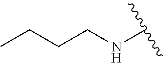 [SMILES: *NCCCC] | OH | H | C |
| 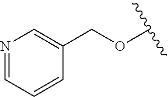 [SMILES: *OCc1cccnc1] | H | H | C |
| 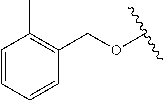 [SMILES: *OCc1ccccc1C] | H | H | C |
| 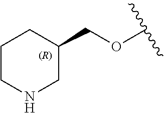 | H | H | C |
| 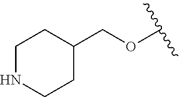 [SMILES: *OCC1CCNCC1] | H | 2-Cl | C |
| 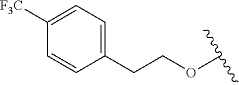 [SMILES: *OCCc1ccc(C(F)(F)F)cc1] | H | H | C |
| 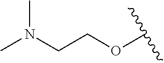 [SMILES: *OCCN(C)C] | — | H | N |
| 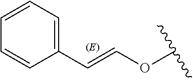 | — | H | N |
| 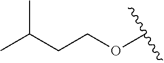 [SMILES: *OCCC(C)C] | — | H | N |

or a pharmaceutically acceptable salt thereof.

69. The compound of claim 58, wherein the compound is:

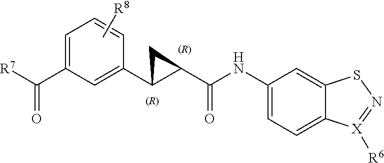

| R⁷ | R⁶ | R⁸ | X |
|---|---|---|---|
| OH | H | H | C |
| OH | — | 3-F | N |
| NH₂ | OH | H | C |
| NH₂ | H | H | C |
| OMe | — | 2-F | N |
| OMe | H | H | C |
| 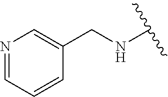 [SMILES: *NCc1cccnc1] | OH | H | C |
| 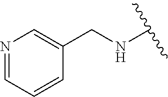 [SMILES: *NCc1cccnc1] | — | H | N |
| 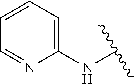 [SMILES: *Nc1ccccn1] | OH | 2-Cl | C |
| 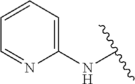 [SMILES: *Nc1ccccn1] | OMe | H | C |
| 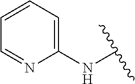 [SMILES: *Nc1ccccn1] | — | H | N |
| 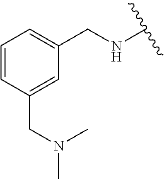 [SMILES: *NCc1cccc(CN(C)C)c1] | H | H | C |
| 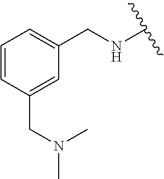 [SMILES: *NCc1cccc(CN(C)C)c1] | — | 3-F | N |
| 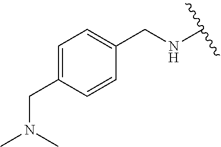 [SMILES: *NCc1ccc(CN(C)C)cc1] | H | H | C |
| 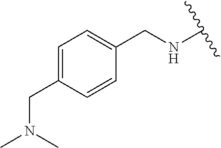 [SMILES: *NCc1ccc(CN(C)C)cc1] | H | H | C |
| 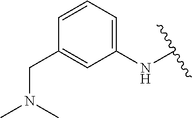 [SMILES: *Nc1cccc(CN(C)C)c1] | — | H | N |
| 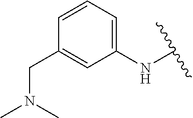 [SMILES: *Nc1cccc(CN(C)C)c1] | H | H | C |
| 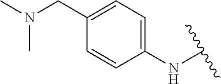 [SMILES: *Nc1ccc(CN(C)C)cc1] | H | 2-F | C |
| 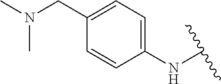 [SMILES: *Nc1ccc(CN(C)C)cc1] | — | H | N |
| 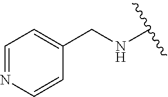 [SMILES: *NCc1ccncc1] | — | H | N |
| 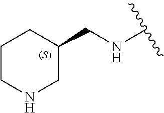 | OH | H | C |
| 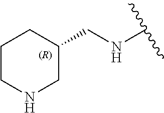 | H | 2-F | C |
| 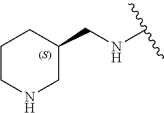 | — | H | N |
| 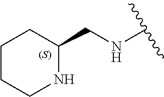 | — | H | N |
| 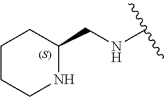 | OH | H | C |
| 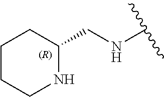 | H | 3-F | C |
| 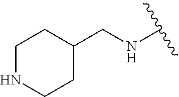 [SMILES: *NCC1CCNCC1] | H | H | C |
| 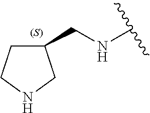 | H | H | C |
| 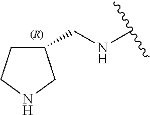 | H | H | C |
| 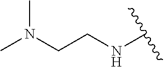 [SMILES: *NCCN(C)C] | H | 3-F | C |
| 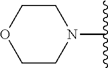 [SMILES: *N1CCOCC1] | — | H | N |
| 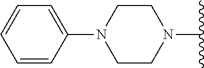 [SMILES: *N1CCN(c2ccccc2)CC1] | — | 2-F | N |
| 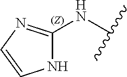 | OH | H | C |
| 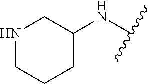 [SMILES: *NC1CCCNC1] | H | H | C |
| 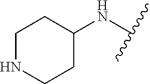 [SMILES: *NC1CCNCC1] | — | H | N |
| 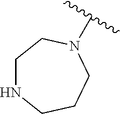 [SMILES: *N1CCCNCC1] | H | H | C |
| 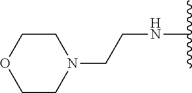 [SMILES: *NCCN1CCOCC1] | H | H | C |
| 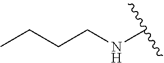 [SMILES: *NCCCC] | OH | H | C |
| 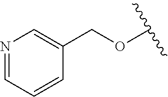 [SMILES: *OCc1cccnc1] | H | H | C |
| 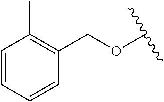 [SMILES: *OCc1ccccc1C] | H | H | C |
| 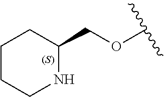 | H | H | C |
| 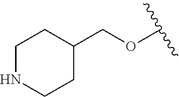 [SMILES: *OCC1CCNCC1] | H | 2-Cl | C |
| 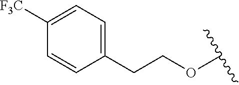 [SMILES: *OCCc1ccc(C(F)(F)F)cc1] | H | H | C |
| 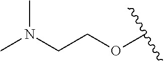 [SMILES: *OCCN(C)C] | — | H | N |
| 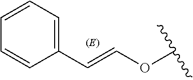 [SMILES: *O/C=C/c1ccccc1] | — | H | N |
| 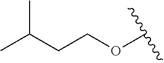 [SMILES: *OCCC(C)C] | — | H | N |
| 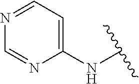 [SMILES: *Nc1ccncn1] | H | H | C |
| 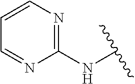 [SMILES: *Nc1ncccn1] | — | H | N |

or a pharmaceutically acceptable salt thereof.

70. The compound of claim 18, wherein the compound of Formula (V) is a compound of Formula (V5): 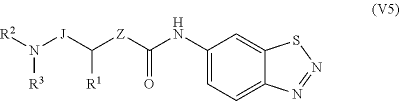
or a pharmaceutically acceptable salt thereof;
wherein
J is a bond, methylene or ethylene;
Z is a bond, methylene or ethylene;
or Z is CH and R³ and Z, together with the atoms to which they are attached, form a C₃₋₆-heterocycloalkyl;
R¹ is hydrogen; halogen; —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen, —C₁₋₆-alkyl, —C₁₋₆-haloalkyl, or —(C₁₋₆-alkyl)-OH; —C₄₋₁₀-heteroaryl; 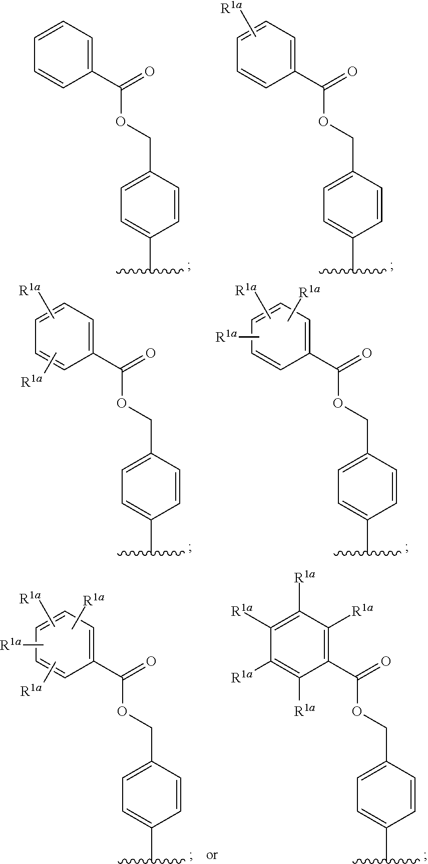
R^{1a} is, independently, halogen, —C₁₋₆-alkyl, or —C₁₋₆-haloalkyl;
R² is hydrogen, —C₁₋₆-alkyl, —(C₁₋₆-alkyl)N(C₁₋₆-alkyl) C₁₋₆-alkyl, or —C(NH)NH₂;
R³ is hydrogen or —C₁₋₆-alkyl;
or R² and R³, together with the atoms to which they are attached, form a C₂₋₆-heterocycloalkyl.

71. The compound of claim 70, wherein R¹ is —C₃₋₁₀-cycloalkyl; —C₆₋₁₀-aryl; —C₆₋₁₀-aryl substituted with halogen, —C₁₋₆-alkyl, —C₁₋₆-haloalkyl, or —(C₁₋₆-alkyl)-OH; —CH₂—(C₆₋₁₀-aryl); —CH₂—(C₆₋₁₀-aryl) substituted with halogen, —C₁₋₆-alkyl, —C₁₋₆-haloalkyl, or —(C₁₋₆-alkyl)-OH; —C₄₋₁₀-heteroaryl;

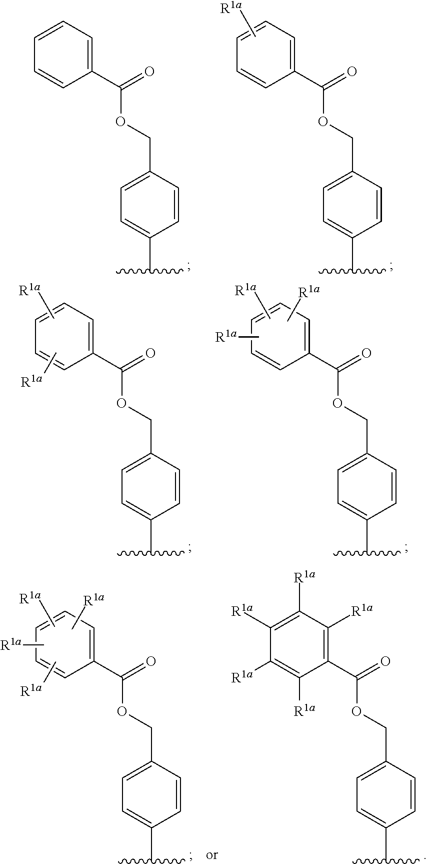

72. The compound of claim 70, wherein R¹ is

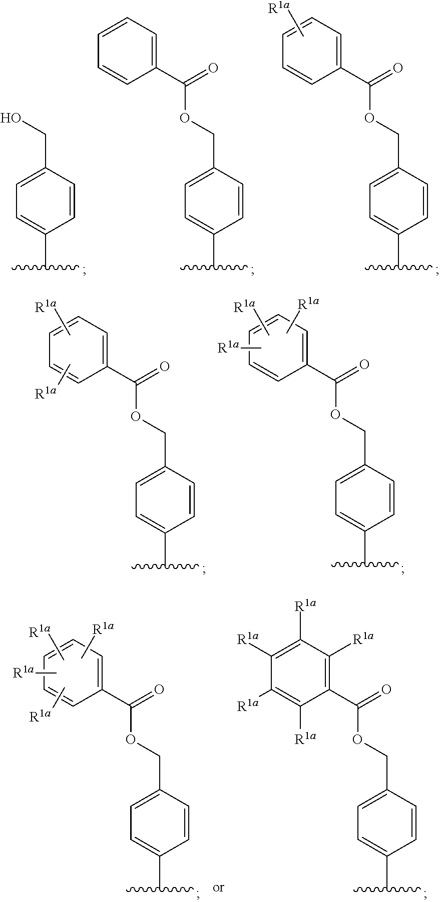

73. The compound of claim 70, wherein R¹ is

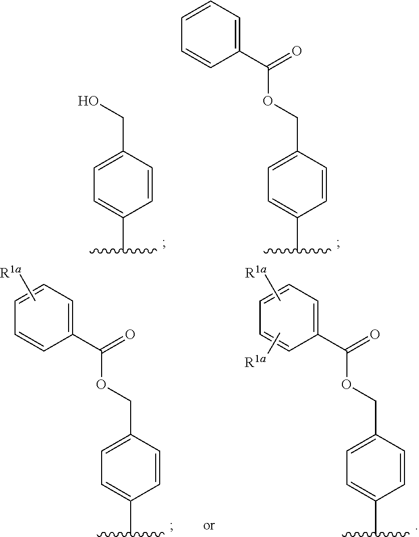

74. The compound of claim 70, wherein R^{1a} is methyl, monohalo-methyl, dihalo-methyl, or trihalo-methyl.

75. The compound of claim 70, wherein R¹ is

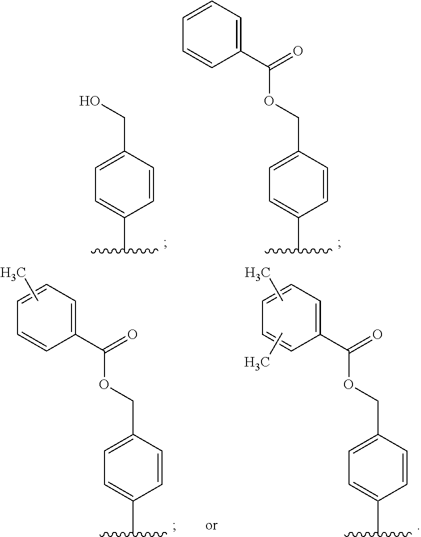

76. The compound of claim 70, wherein R² and R³, together with the atoms to which they are attached, form an azetidinyl, pyrrolidinyl, piperidinyl, azepanyl, diazetidinyl, imidazolidinyl, piperazinyl, diazepanyl, oxazetidinyl, oxazolidinyl, morpholinyl, or oxazepanyl.

77. The compound of claim 70, wherein Z is CH and R³ and Z, together with the atoms to which they are attached, form a C₃₋₆-heterocycloalkyl.

78. The compound of claim 70, wherein the compound of Formula (V5) is a compound of Formula (V5a): 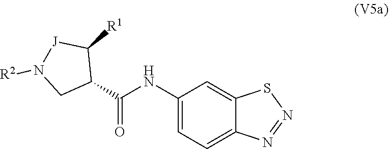
or a pharmaceutically acceptable salt thereof.

79. The compound of claim 70, wherein Z is CH and R³ and Z, together with the atoms to which they are attached, form a pyrrolidinyl.

80. The compound of claim 70, wherein the compound of Formula (V5) is a compound of Formula (V5b): 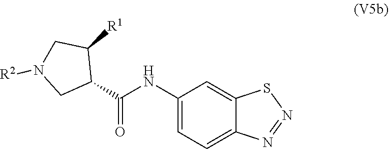
or a pharmaceutically acceptable salt thereof.

81. The compound of claim 70, wherein R^{1a} is, independently, F, Cl, Br, —C₁₋₃-alkyl, or —C₁₋₃-haloalkyl.

82. The compound of claim 70, wherein R^{1a} is, independently, F, Cl, methyl, monohalo-methyl, dihalo-methyl, or trihalo-methyl.

83. The compound of claim 70, wherein R² is hydrogen, —C₁₋₃-alkyl, —(C₁₋₃-alkyl)N(C₁₋₃-alkyl) C₁₋₃-alkyl, —(C₁₋₃-alkyl)N(H) C₁₋₃-alkyl, —(C₁₋₃-alkyl)NH₂, or —C(NH)NH₂.

84. The compound of claim 70, wherein R² is hydrogen, methyl, —(C₁₋₃-alkyl)N(CH₃) CH₃, —(C₁₋₃-alkyl)N(H) CH₃, —(C₁₋₃-alkyl)NH₂, or —C(NH)NH₂.

85. The compound of claim 70, wherein R³ is hydrogen or —C₁₋₃-alkyl.

86. The compound of claim 70, wherein R³ is hydrogen or methyl.

87. The compound of claim 70, wherein R¹ is —C₃₋₆-cycloalkyl; phenyl; -phenyl substituted with halogen, —C₁₋₆-alkyl, —C₁₋₆-haloalkyl, or —(C₁₋₆-alkyl)-OH; benzyl; benzyl substituted with halogen, —C₁₋₆-alkyl, —C₁₋₆-haloalkyl, or —(C₁₋₆-alkyl)-OH; or —C₄₋₆-heteroaryl.

88. The compound of claim 112, wherein R¹ is —C₃₋₆-cycloalkyl; phenyl; -phenyl substituted with halogen, —C₁₋₃-alkyl, —C₁₋₃-haloalkyl, or —(C₁₋₃-alkyl)-OH; benzyl; benzyl substituted with halogen, —C₁₋₃-alkyl, —C₁₋₃-haloalkyl, or —(C₁₋₃-alkyl)-OH; or —C₄₋₅-heteroaryl.

89. The compound of claim 70, wherein J is ethylene and Z is a bond.

90. The compound of claim 70, wherein J is a bond and Z is ethylene.

91. The compound of claim 70, wherein J is methylene and Z is a bond.

92. The compound of claim 70, wherein J is a bond and Z is methylene.

93. The compound of claim 70, wherein J is methylene and Z is methylene.

94. The compound of claim 70, wherein J is a bond and Z is a bond.

95. The compound of claim 18, wherein the compound is 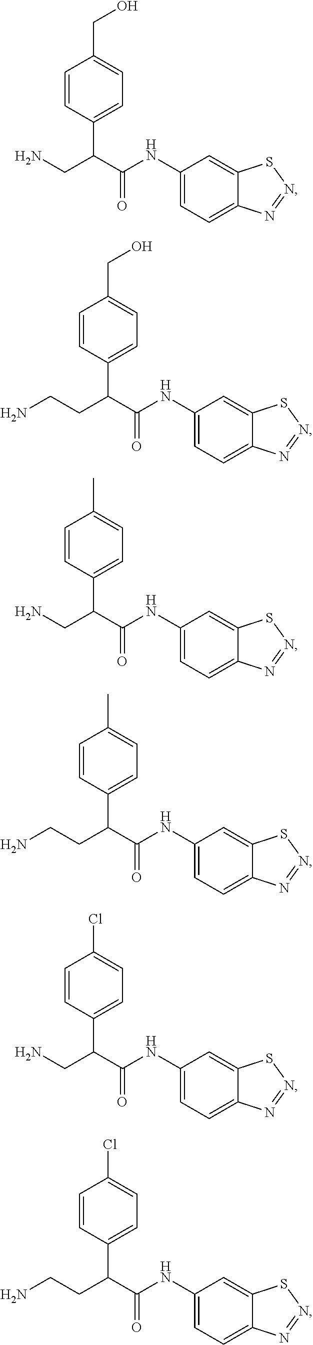 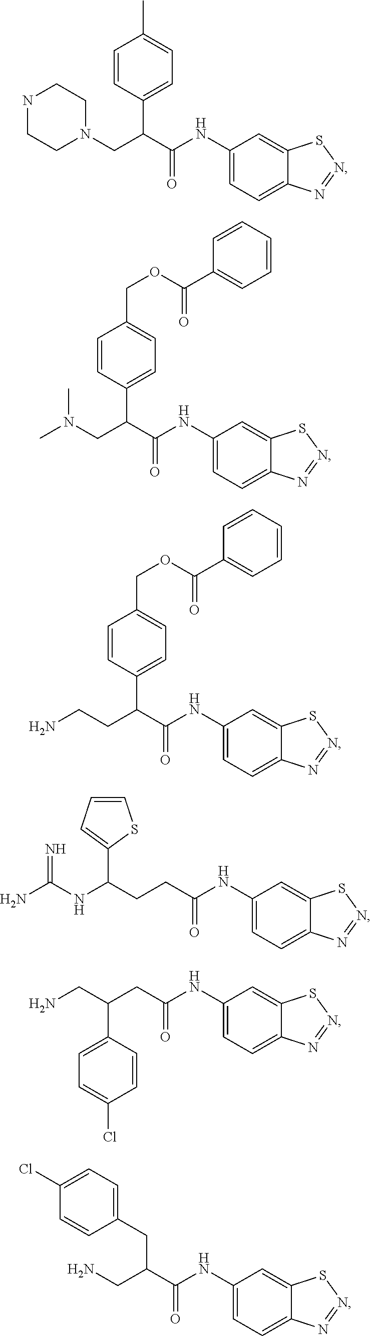 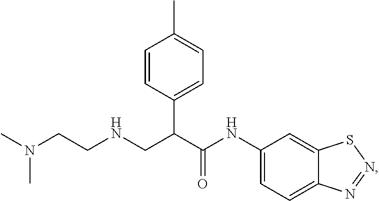 [SMILES: Cc1ccc(C(CNCCN(C)C)C(=O)Nc2ccc3nnsc3c2)cc1]
or a pharmaceutically acceptable salt thereof.

96. The compound of claim 18, wherein the compound is 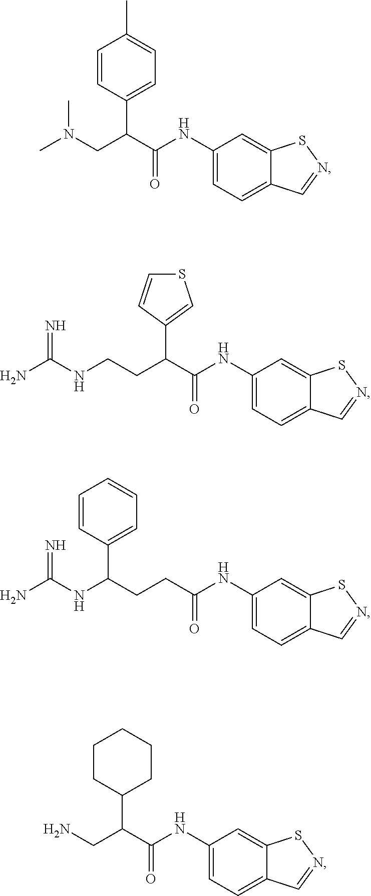 [SMILES: Cc1ccc(C(CN(C)C)C(=O)Nc2ccc3cnsc3c2)cc1 ; N=C(N)NCCC(C(=O)Nc1ccc2cnsc2c1)c1ccsc1 ; N=C(N)NC(CCC(=O)Nc1ccc2cnsc2c1)c1ccccc1 ; NCC(C(=O)Nc1ccc2cnsc2c1)C1CCCCC1]
or a pharmaceutically acceptable salt thereof.

97. The compound of claim 18, wherein the compound is 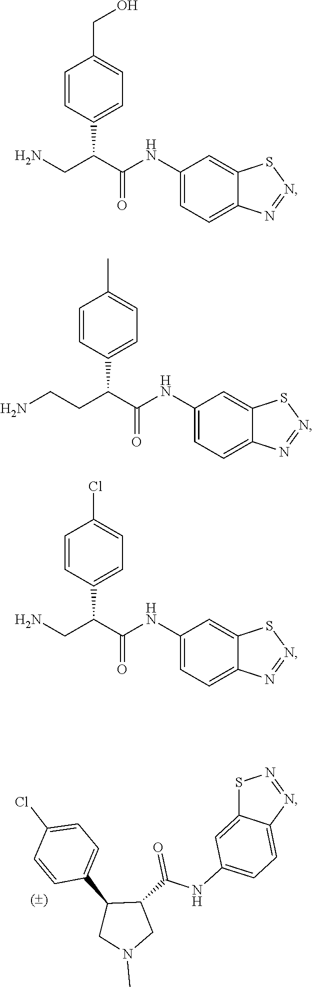 [SMILES: NC[C@@H](C(=O)Nc1ccc2nnsc2c1)c1ccc(CO)cc1 ; Cc1ccc([C@@H](CCN)C(=O)Nc2ccc3nnsc3c2)cc1 ; NC[C@@H](C(=O)Nc1ccc2nnsc2c1)c1ccc(Cl)cc1 ; CN1C[C@@H](C(=O)Nc2ccc3nnsc3c2)[C@H](c2ccc(Cl)cc2)C1]
or a pharmaceutically acceptable salt thereof.

98. The compound of claim 18, wherein the compound is 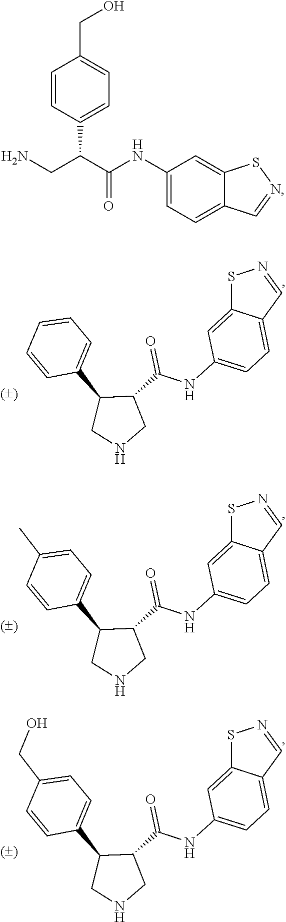 [SMILES: NC[C@@H](C(=O)Nc1ccc2cnsc2c1)c1ccc(CO)cc1 ; O=C(Nc1ccc2cnsc2c1)[C@@H]1CNC[C@H]1c1ccccc1 ; Cc1ccc([C@@H]2CNC[C@H]2C(=O)Nc2ccc3cnsc3c2)cc1 ; O=C(Nc1ccc2cnsc2c1)[C@@H]1CNC[C@H]1c1ccc(CO)cc1]
or a pharmaceutically acceptable salt thereof.

99. A compound, wherein the compound is 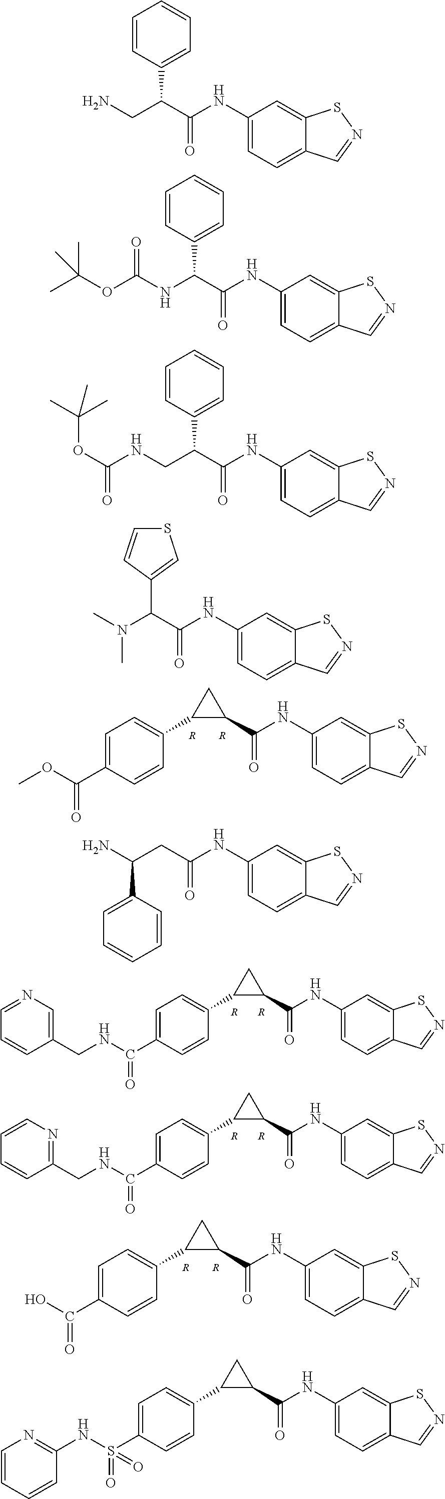 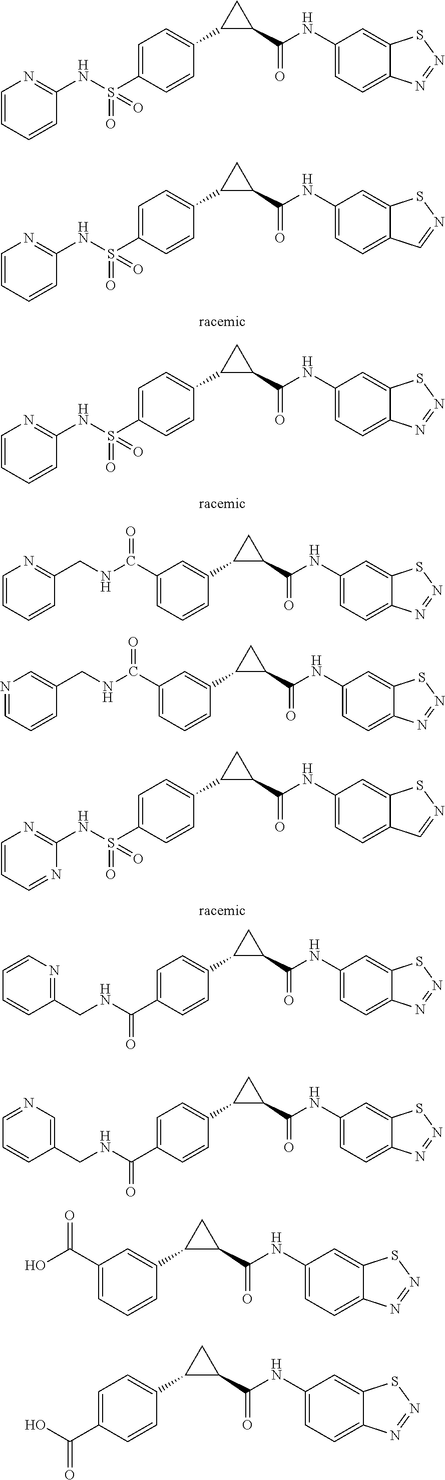 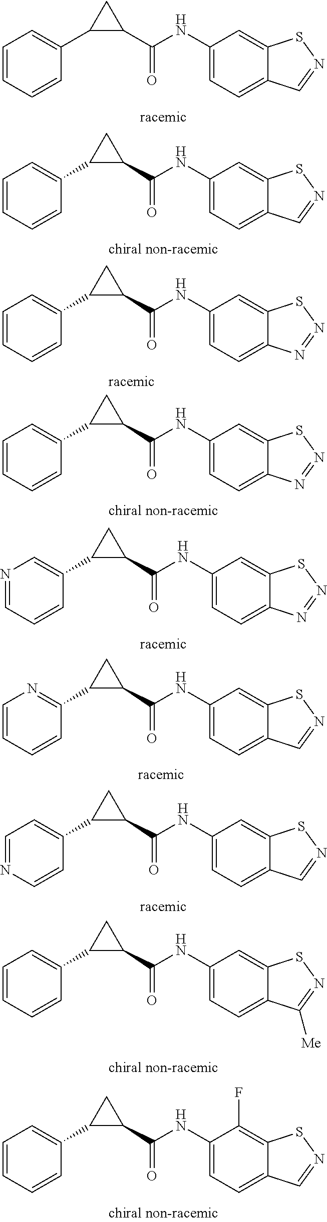
or a pharmaceutically acceptable salt thereof.

100. A composition comprising the compound of any of claims 1-99.

101. A pharmaceutical composition comprising the compound of any of claims 1-99 and a pharmaceutically acceptable carrier.

102. The pharmaceutical composition of claim 101, wherein the carrier is saline buffered to a pH of about 5.5 to about 6.5.

103. The pharmaceutical composition of claim 101, wherein the carrier is saline buffered to a pH of about 4.5 to about 5.5.

104. The pharmaceutical composition of claim 101, wherein the carrier is saline buffered to a pH of about 4.9 to about 5.1.

105. A kit comprising the compound of any of claims 1-99, and instructions for use thereof.

106. A kit comprising the pharmaceutical composition of any of claims 101-104, and instructions for use thereof.

107. An article of manufacture comprising the compound of any of claims 1-99.

108. An article of manufacture comprising the pharmaceutical composition of any of claims 101-104, and instructions for use thereof.

109. A method of treating a disease in a subject in need thereof, comprising administering to the subject an effective amount of the compound of any of claims 1-99.

110. The method of claim 109, wherein the disease comprises at least one of eye disease, bone disorder, obesity, heart disease, inflammatory disease, hepatic disease, renal disease, pancreatitis, cancer, myocardial infarct, gastric disturbance, hypertension, fertility control, disorders of hair growth, nasal congestion, neurogenic bladder disorder, gastrointestinal disorder, or dermatological disorder.

111. The method of claim 109, wherein the disease comprises an eye disease.

112. The method of claim 109, wherein the eye disease comprises glaucoma or a neurodegenerative eye disease.

113. The method of claim 109, wherein the disease is an eye disease.

114. The method of claim 109, wherein the eye disease is glaucoma, a neurodegenerative eye disease, dry eye, or ocular hypertension.

115. A method of modulating kinase activity in a cell, comprising contacting the cell with the compound of any of claims 1-99 in an amount effect to modulate kinase activity.

116. The method of claim 115, wherein the cell is in a subject.

117. The method of claim 116, wherein the subject is a human.

118. A method of reducing intraocular pressure in a subject in need thereof, comprising contacting the subject with an effective amount of the compound of any of claims 1-99.

119. The method of any of claims 109-118, wherein the compound is administered to an eye of the subject.

120. The method of any of claims 109-118, wherein the compound is administered topically to an eyelid of the subject.

121. The method of any of claims 109-118, wherein the compound is administered systemically to the subject.

122. The method of any of claims 109-118, wherein the compound is administered topically to the subject.
